(19) United States
(12) Patent Application Publication
(10) Pub. No.: US 2006/0093613 A1
(43) Pub. Date: May 4, 2006

(54) Soluble T cell receptor

(75) Inventors: Bent Karsten Jakobsen, Wantage (GB); John Irving Bell, Wallingford (GB); George Fu Gao, Brookline, MA (US); Benjamin Ernest Willcox, Oxford (GB); Jonathan Michael Boulter, Oxford (GB)

Correspondence Address: WILMER CUTLER PICKERING HALE AND DORR LLP, 60 STATE STREET, BOSTON, MA, 02109, US

(73) Assignee: AVIDEX LIMITED, Farmoor (GB)

(21) Appl. No.: 11/167,773

(22) Filed: Jun. 27, 2005

Related U.S. Application Data
(63) Continuation of application No. 10/014,326, filed on Nov. 13, 2001, now abandoned.
Application No. 10/014,326 is a continuation of application No. 09/335,087, filed on Jun. 17, 1999, now abandoned.

(30) Foreign Application Priority Data
May 19, 1999 (WO) PCT/GB99/01588
May 19, 1998 (GB) 9810759.2
Sep. 29, 1998 (GB) 9821129.5

(51) Int. Cl. C07K 14/74; C07H 21/04; C12P 21/06; A61K 39/00; C12N 15/74

(52) U.S. Cl. 424/185.1; 435/69.1; 435/252.3; 435/471; 530/350; 536/23.5

(57) ABSTRACT

The present invention relates to a recombinant soluble T cell receptor. The T cell receptor (TCR) is refolded and comprises a recombinant TCR α or γ chain extracellular domain having a first heterologous C-terminal dimerization peptide; and a recombinant TCR β or δ chain extracellular domain having a second C-terminal dimerization peptide which is specifically heterodimerized with the first dimerization peptide to form a heterodimerization domain, which may be a coiled coil domain. The invention also provides nucleic acid sequences encoding the recombinant TCR and a method for producing the recombinant TCR. The TCR may be labelled with a detectable label so as to enable the detection of specific MHC-peptide complexes. Alternatively, it can be linked to a therapeutic agent such as a cytotoxic agent or an immunostimulating agent so as to deliver such an agent to the site of a specific MHC-peptide complex.

### RELATED APPLICATIONS

This application is a continuation of U.S. Ser. No. 10/014,326, filed Nov. 13, 2001, which is a continuation of U.S. Ser. No. 09/335,087, filed Jun. 17, 1999, abandoned, which was the U.S. national phase of International Patent Application No. PCT GB/99/01588, filed May 19, 1999, which claimed priority to Great Britain Patent Application Nos. 9821129.5, filed Sep. 29, 1998, and 9810759.2, filed May 19, 1998.

### FIELD OF THE INVENTION

This invention relates to non-membrane bound recombinant T cell receptors (TCRS) and to methods and reagents for producing them.

### GENERAL BACKGROUND

1. Antigen Presentation on the Cell Surface

MHC molecules are specialized protein complexes which present short protein fragments, peptide antigens, for recognition on the cell surface by the cellular arm of the adaptive immune system.

Class I MHC is a dimeric protein complex consisting of a variable heavy chain and a constant light chain, β2 microglobulin. Class I MHC presents peptides which are processed intracellularly, loaded into a binding cleft in the MHC, and transported to the cell surface where the complex is anchored in the membrane by the MHC heavy chain. Peptides are usually 8-11 amino acids in length, depending on the degree of arching introduced in the peptide when bound in the MHC. The binding cleft, which is formed by the membrane distal α1 and α2 domains of the MHC heavy chain, has “closed” ends, imposing quite tight restrictions on the length of peptide which can be bound.

Class II MHC is also a dimeric protein consisting of an α (heavy) and a β (light) chain, both of which are variable glycoproteins and anchored in the cell by transmembrane domains. Like Class I MHC, the Class II molecule forms a binding cleft in which longer peptides of 12-24 amino acids are inserted. Peptides are taken up from the extracellular environment by endocytosis and processed before loading into the Class II complex, which is then transported to the cell surface.

Each cell presents peptides in up to six different Class I molecules and a similar number of Class II molecules, the total number of MHC complexes presented being in the region 10⁵-10⁶ per cell. The diversity of peptides presented in Class I molecules is typically estimated to be between 1,000-10,000, with 90% of these being present in 100-1,000 copies per cell (Hunt, Michel et al. 1992; Chicz, Urban et al. 1993; Engelhard, Appella et al. 1993; Huczko, Bodnar et al. 1993). The most abundant peptides are thought to constitute between 0.4-5% of the total peptide presented, which means that up to 20,000 identical complexes could be present. However, an average number for the most abundant single peptide complexes is likely to be in the region of 2,000-4,000 per cell, and typical presentation levels of recognizable T cell epitopes are in the region of 100-500 complexes per cell (for review see (Engelhard 1994)).

2. Recognition of Antigen Presenting Cells

A wide spectrum of cells can present antigen, as MHC-peptide, and the cells that have that property are known as antigen presenting cells (APC). The type of cell which presents a particular antigen depends upon how and where the antigen first encounters cells of the immune system. APCs include the interdigitating dendritic cells found in the T cell areas of the lymph nodes and spleen in large numbers; Langerhans cells in the skin; follicular dendritic cells in B cell areas of the lymphoid tissue; monocytes, macrophages and other cells of the monocyte/macrophage lineage; B cells and T cells; and a variety of other cells such as endothelial cells and fibroblasts which are not classical APCs but can act in the manner of an APC.

APCs are recognized by a subgroup of lymphocytes which mature in the thymus (T cells), where they undergo a selection procedure designed to ensure that T cells which respond to self-peptides are eradicated (negative selection). In addition, T cells which do not have the ability to recognize the MHC variants which are presented (in man, the HLA haplotypes) fail to mature (positive selection).

Recognition of specific MHC-peptide complexes by T cells is mediated by the T cell receptor (TCR) which consists of an α- and a β-chain, both of which are anchored in the membrane. In a recombination process similar to that observed for antibody genes, the TCR α and β genes rearrange from Variable, Joining, Diversity and Constant elements creating enormous diversity in the extracellular antigen binding domains (10¹³ to 10¹⁵ different possibilities). TCRs also exist in a different form with γ and δ chains, but these are only present on about 5% of T cells.

Antibodies and TCRs are the only two types of molecules which recognize antigens in a specific manner, and thus the TCR is the only receptor specific for particular peptide antigens presented in MHC, the alien peptide often being the only sign of an abnormality within a cell.

TCRs are expressed in enormous diversity, each TCR being specific for one or a few MHC-peptide complexes. Contacts between TCR and MHC-peptide ligands are extremely short-lived, usually with a half-life of less than a second. Adhesion between T cells and target cells, presumably TCR/MHC-peptide, relies on the employment of multiple TCR/MHC-peptide contacts as well as a number coreceptor-ligand contacts.

T cell recognition occurs when a T-cell and an antigen presenting cell (APC) are in direct physical contact and is initiated by ligation of antigen-specific TCRs with pMHC complexes. The TCR is a heterodimeric cell surface protein of the immunoglobulin superfamily which is associated with invariant proteins of the CD3 complex involved in mediating signal transduction. TCRs exist in αβ and γδ forms, which are structurally similar but have quite distinct anatomical locations and probably functions. The extracellular portion of the receptor consists of two membrane-proximal constant domains, and two membrane-distal variable domains bearing highly polymorphic loops analogous to the complementarity determining regions (CDRs) of antibodies. It is these loops which form the MHC-binding site of the TCR molecule and determine peptide specificity. The MHC class I and class 11 ligands are also immunoglobulin superfamily proteins but are specialized for antigen presentation, with a highly polymorphic peptide binding site which enables them to present a diverse array of short peptide fragments at the APC cell surface.

Recently, examples of these interactions have been characterized structurally (Garboczi, Ghosh et al. 1996; Garcia, Degano et al. 1996; Ding, Smith et al. 1998). Crystallographic structures of murine and human Class I pMHC-TCR complexes indicate a diagonal orientation of the TCR over its pMHC ligand and show poor shape complementarity in the interface. CDR3 loops contact exclusively peptide residues. Comparisons of liganded and unliganded TCR structures also suggest that there is a degree of flexibility in the TCR CDR loops (Garboczi and Biddison 1999).

T cell activation models attempt to explain how such protein-protein interactions at an interface between T cell and antigen presenting cell (APC) initiate responses such as killing of a virally infected target cell. The physical properties of TCR-pMHC interactions are included as critical parameters in many of these models. For instance, quantitative changes in TCR dissociation rates have been found to translate into qualitative differences in the biological outcome of receptor engagement, such as full or partial T cell activation, or antagonism (Matsui, Boniface et al. 1994; Rabinowitz, Beeson et al. 1996; Davis, Boniface et al. 1998).

TCR-pMHC interactions have been shown to have low affinities and relatively slow kinetics. Many studies have used biosensor technology, such as BIACORE™ (Willcox, Gao et al. 1999; Wyer, Willcox et al. 1999), which exploits surface plasmon resonance (SPR) and enables direct affinity and real-time kinetic measurements of protein-protein interactions (Garcia, Scott et al. 1996; Davis, Boniface et al. 1998). However, the receptors studied are either alloreactive TCRs or those which have been raised in response to an artificial immunogen.

3. TCR and CD8 Interactions with MHC-Peptide Complexes

The vast majority of T cells restricted by Class I MHC-peptide complexes also require the engagement of the coreceptor CD8 for activation, while T cells restricted by Class II MHC require the engagement of CD4. The exact function of the coreceptors in T cell activation is not yet entirely clarified. Neither are the critical mechanisms and parameters controlling activation. However, both CD8 and CD4 have cytoplasmic domains which are associated with the kinase p56^{1ck} which is involved in the very earliest tyrosine phosphorylation event which characterizes T cell activation. CD8 is a dimeric receptor, expressed either in an αα form or, more commonly, in an αβ form. CD4 is a monomer. In the CD8 receptor, only the α-chain is associated with p56^{1ck}.

Recent determinations of the physical parameters controlling binding of TCR and CD8 to MHC, using soluble versions of the receptors, have shown that binding by TCR dominates the recognition event. TCR has significantly higher affinity for MHC than the coreceptors (Willcox, Gao et al. 1999; Wyer, Willcox et al. 1999).

The individual interactions of the receptors with MHC are very short-lived at physiological temperature, i.e. 37° C. An approximate figure for the half-life of a TCR-MHC/peptide interaction, measured with a human TCR specific for the influenza virus “matrix” peptide presented by HLA-A*0201 (HLA-A2), is 0.7 seconds. The half-life of the CD8αα interaction with this MHC/peptide complex is less than 0.01 seconds, i.e. at least 18 times faster.

4. Production of Soluble MHC-Peptide Complexes

Soluble MHC-peptide complexes were first obtained by cleaving the molecules on the surface of antigen presenting cells with papain (Bjorkman, Strominger et al. 1985). Although this approach provided material for crystallization, it has, for Class I molecules, in recent years been replaced by individual expression of heavy and light chain in E. coli followed by refolding in the presence of synthetic peptide (Garboczi, Hung et al. 1992; Madden, Garboczi et al. 1993; Garboczi, Madden et al. 1994; Reid, McAdam et al. 1996; Reid, Smith et al. 1996; Smith, Reid et al. 1996; Smith, Reid et al. 1996; Gao, Tormo et al. 1997; Gao, Gerth et al. 1998). This approach has several advantages over previous methods in that a better yield is obtained at a lower cost, peptide identity can be controlled very accurately, and the final product is more homogeneous. Furthermore, expression of modified heavy or light chain, for instance fused to a protein tag, can be easily performed.

5. Soluble TCR

At present, there are no published data on human TCR-pMHC interactions, and no studies analyzing naturally selected TCRs specific for natural (e.g. viral) epitopes. This may reflect difficulties in obtaining protein which is suitable for SPR, i.e. protein which is homogenous, monomeric, correctly folded, available in milligram quantities and stable over a range of concentration.

It would be an advantage to be able to produce a recombinant TCR in non-membrane bound (or soluble) form, not only for the purpose of investigating specific TCR-pMHC interactions, but also as a diagnostic tool to detect infection or to detect autoimmune disease markers, or to detect the efficacy of T cell vaccines. Soluble TCR would also have applications in staining, for example to stain cells for the presence of a particular viral antigen presented in the context of the MHC. Similarly, the soluble TCR could be used to deliver a therapeutic agent, for example a cytotoxic compound or an immunostimulating compound, to cells presenting a particular antigen.

Proteins which are made up of more than one polypeptide subunit and which have a transmembrane domain can be difficult to produce in soluble form because in many cases the protein is stabilized by its transmembrane region. This is the case for the TCR.

Production of soluble TCR has only recently been described by a number of groups. In general, all methods describe truncated forms of TCR, containing either only extracellular domains or extracellular and cytoplasmic domains. Thus, in all cases, the transmembrane domains have been deleted from the expressed protein. Although many reports show that TCR produced according to their methods can be recognized by TCR-specific antibodies (indicating that the part of the recombinant TCR recognized by the antibody has correctly folded), none has been able to produce a soluble TCR at a good yield which is stable at low concentrations and which can recognize MHC-peptide complexes.

The first approach to yield crystallizable material made use of expression in eukaryotic cells but the material is extremely expensive to produce (Garcia, Degano et al. 1996; Garcia, Scott et al. 1996). Another approach which has produced crystallizable material made use of an E. coli expression system similar to what has previously been used for MHC-peptide complexes (Garboczi, Ghosh et al. 1996; Garboczi, Utz et al. 1996). The latter method, which expresses the extracellular portions of the TCR chains truncated immediately before the cysteine residues involved in forming the interchain disulfide bridge, followed by refolding in vitro, has turned out not to be generally applicable. Most heterodimeric TCRs appear to be unstable when produced in this fashion due to low affinity between the α and γ chains.

In addition, a number of other descriptions of engineered production of soluble TCR exist. Some of these describe only the expression of either the α or β chain of the TCR, thus yielding protein which does not retain MHC-peptide specific binding (Calaman, Carson et al. 1993; Ishii, Nakano et al. 1995). β chain crystals have been obtained without a chain, either alone or bound to superantigen (Boulot, Bentley et al. 1994; Bentley, Boulot et al. 1995; Fields, Malchiodi et al. 1996).

Other reports describe methods for expression of heterodimeric γ/δ or α/β TCR (Gregoire, Rebai et al. 1991; Necker, Rebai et al. 1991; Eilat, Kikuchi et al. 1992; Weber, Traunecker et al. 1992; Corr, Slanetz et al. 1994; Ishii, Nakano et al. 1995; Gregoire, Malissen et al. 1996; Romagne, Peyrat et al. 1996). In some cases, the TCR has been expressed as a single chain fusion protein (Brocker, Peter et al. 1993; Gregoire, Malissen et al. 1996; Schlueter, Schodin et al. 1996). Another strategy has been to express the TCR chains as chimeric proteins fused to Ig hinge and constant domains (Eilat, Kikuchi et al. 1992; Weber, Traunecker et al. 1992). Other chimaeric TCR proteins have been expressed with designed sequences which form coiled-coils which have high affinity and specificity for each other, thus stabilizing TCR α-β contacts and increasing solubility. This approach was taken by Chang, Bao et al. (1994) who replaced the transmembrane region of the protein with a leucine zipper protein consisting of two synthetic peptide sequences, an acid peptide and a base peptide, that specifically interact to create a heterodimeric coiled coil. The authors employed a bacculovirus expression system in eukaryotic cells to secrete heterodimeric TCR protein. The artificial leucine zipper peptides assist heterodimerization of the TCR α and β chains, which are also linked by an interchain disulfide bond just above the fusion point with the zipper peptides. However, these techniques have not since proved successful and there is no evidence that the soluble TCR described can recognize a TCR ligand. Similarly, Golden, Khandekar et al. (1997) described the production of heterodimeric T cell receptors as leucine zipper fusion proteins. The soluble TCR was expressed in E. coli as a secreted heterodimer with the α-β interchain disulfide bond as in Chang et al. (1994). Again, there is no evidence that this ready-folded TCR heterodimer is capable of interacting with its ligand.

There is therefore a need for a soluble version of the membrane bound TCR, which is correctly folded so that it is capable of recognizing its native ligand. A soluble form of a TCR which is stable over a period of time, and a method for producing it in reasonable quantities, would also be useful. The present invention aims to meet some or all of these requirements.

The Invention

The invention provides in one aspect a refolded recombinant T cell receptor (TCR) which comprises:

i) a recombinant TCR α or γ chain extracellular domain having a first heterologous C-terminal dimerization peptide; and

ii) a recombinant TCR βor δ chain extracellular domain having a second C-terminal dimerization peptide which is specifically heterodimerized with the first dimerization peptide to form a heterodimerization domain.

The TCR according to the invention, which is refolded after being expressed, rather than secreted as a heterodimer, is conformationally superior to soluble TCR previously available. This is indicated by its ability to recognize MHC-peptide complexes. It is also stable at relatively low concentrations. It may be stable at concentrations below 1 mg/ml and preferably about 10 μg/ml.

In a further aspect, the invention provides a biologically-active recombinant T cell receptor (TCR) which comprises:

i) a recombinant TCR α or γ chain extracellular domain having a first heterologous C-terminal dimerization peptide; and

ii) a recombinant TCR β or δ chain extracellular domain having a second C-terminal dimerization peptide which is specifically heterodimerized with the first dimerization peptide to form a heterodimerization domain. Such a TCR will bind specifically to MHC-peptide complexes, preferably in a manner similar to the native TCR from which it is derived.

In another aspect, the invention provides recombinant nucleic acid sequences encoding the recombinant TCR chains as described herein. Such nucleic acid sequences may be isolated from T-cell clones. Alternatively, they may be produced synthetically, for example by inserting a heterologous nucleic acid sequence in a nucleic acid sequence encoding a TCR chain, or by mutating (by insertion, deletion or substitution) a nucleic acid sequence encoding a TCR chain. Thus, the invention includes within its scope synthetic peptides which have the activity and/or binding specificity of native TCRs.

In yet another aspect, the invention provides a method of making a recombinant non membrane bound T cell receptor, which method comprises:

expressing a recombinant TCR α or γ chain extracellular domain having a first heterologous C-terminal dimerization peptide; and a recombinant TCR β or δ chain extracellular domain having a second C-terminal dimerization peptide which specifically heterodimerizes with the first dimerization peptide to form a heterodimerization domain; and

refolding the chains together in vitro to produce a TCR heterodimer. Recombinant TCRs in accordance with the invention may be for recognizing Class I MHC-peptide complexes and Class II MHC-peptide complexes.

The heterodimerization domain of the recombinant TCR according to the invention is preferably a so-called “coiled coil” or “leucine zipper”. These terms are used to describe pairs of helical peptides which interact with each other in a specific fashion to form a heterodimer. The interaction occurs because there are complementary hydrophobic residues along one side of each zipper peptide. The nature of the peptides is such that the formation of heterodimers is very much more favorable than the formation of homodimers of the helices. Leucine zippers may be synthetic or naturally occurring. Synthetic leucines can be designed to have a much higher binding affinity than naturally occurring leucine zippers, which is not necessarily an advantage. In fact, preferred leucine zippers for use in the invention are naturally occurring leucine zippers or leucine zippers with a similar binding affinity. Leucine zippers from the c-jun and c-fos protein are an example of leucine zippers with a suitable binding affinity. Other suitable leucine zippers include those from the myc and max proteins (Amati, Dalton, et al. 1992). Other leucine zippers with suitable properties could easily be designed (O'Shea et al. 1993).

It is preferred that the soluble TCRs in accordance with the invention have approximately 40 amino acid leucine zipper fusions corresponding to the heterodimerization domains from c-jun (αchain) and c-fos (βchain) (O'Shea, Rutkowski et al. 1989, O'Shea, Rutkowski et al. 1992, Glover and Harrison, 1995). Longer leucine zippers may be used. Since heterodimerization specificity appears to be retained even in quite short fragments of some leucine zipper domains (O'Shea, Rutkowski et al. 1992), it is possible that a similar benefit could be obtained with shorter c-jun and c-fos fragments. Such shorter fragments could have as few as 8 amino acids for example. Thus, the leucine zipper domains may be in the range of 8 to 60 amino acids long.

The molecular principles of specificity in leucine zipper pairing is well characterized (Landschulz, Johnson et al. 1988; McKnight, 1991) and leucine zippers can be designed and engineered by those skilled in the art to form homodimers, heterodimers or trimeric complexes (Lumb and Kim, 1995; Nautiyal, Woolfson et al. 1995; Boice,. Dieckmann et al. 1996, Chao, Houston et al. 1996). Designed leucine zippers, or other heterodimerization domains, of higher affinity than the c-jun and c-fos leucine zippers may be beneficial for the expression of soluble TCRs in some systems. However, as mentioned in more detail below, when soluble TCR is folded in vitro, a solubilizing agent is preferably included in the folding buffer to reduce the formation of unproductive protein aggregates. One interpretation of this phenomenon is that the kinetics of folding of the leucine zipper domains are faster than for the TCR chains, leading to dimerization of unfolded TCR α and β chain, in turn causing protein aggregation. By slowing the folding process and inhibiting aggregation by inclusion of solubilizing agent, the protein can be maintained in solution until folding of both fusion domains is completed. Therefore, heterodimerization domains of higher affinity than the c-fos and c-jun leucine zippers may require higher concentrations of solubilizing agent to achieve a yield of soluble TCRs comparable to that for c-jun and c-fos.

Different biological systems use a variety of methods to form stable homo- and hetero protein dimers, and each of these methods in principle provide an option for engineering dimerization domains into genetically modified proteins. Leucine zippers (Kouzarides and Ziff 1989) are probably the most popular dimerization modules and have been widely used for production of genetically designed dimeric proteins. Thus, the leucine zipper of GCN4, a transcriptional activator protein from the yeast Saccharomyces cerevisiae, has been used to direct homodimerization of a number of heterologous proteins (Hu, Newell et al. 1993; Greenfield, Montelione et al. 1998). The preferred strategy is to use zippers that direct formation of heterodimeric complexes such as the Jun/Fos leucine zipper pair (de Kruif and Logtenberg 1996; Riley, Ralston et al. 1996).

The heterodimerization domain of the recombinant TCR according to the present invention is not limited to leucine zippers. Thus, it may be provided by disulfide bridge-forming elements. Alternatively, it may be provided by the SH3 domains and hydrophobic/proline rich counterdomains, which are responsible for the protein-protein interactions seen among proteins involved in signal transduction (reviewed in Schlessinger 1994). Other natural protein-protein interactions found among proteins participating in signal transduction cascades rely on associations between post-translationally modified amino acids and protein modules that specifically recognize such modified residues. Such post-translationally modified amino acids and protein modules may form the heterodimerization domain of the recombinant TCR in accordance with the invention. An example of a protein pair of this type is provided by tyrosine phosphorylated receptors such as Epidermal Growth Factor Receptor or Platelet Derived Growth Factor Receptor and the SH2 domain of GRB2 (Lowenstein, Daly et al. 1992; Buday and Downward 1993). As in all fields of science, new dimerization modules are being actively sought (Chevray and Nathans 1992) and methods for engineering completely artificial modules have now successfully been developed (Zhang, Murphy et al. 1999).

In a preferred recombinant TCR according to the invention, an interchain disulfide bond which forms between two cysteine residues in the native α and β TCR chains and between the native γ and δ TCR chains, is absent. This may be achieved for example by fusing the dimerization domains to the TCR receptor chains above the cysteine residues so that these residues are excluded from the recombinant protein. In an alternative example, one or more of the cysteine residues is replaced by another amino acid residue which is not involved in disulfide bond formation. These cysteine residues may not be incorporated because they may be detrimental to in vitro folding of functional TCR.

Refolding of the α and β chains or γ and δ chains of the refolded recombinant TCR according to the invention takes place in vitro under suitable refolding conditions. In a particular embodiment, a recombinant TCR with correct conformation is achieved by refolding solubilized TCR chains in a refolding buffer comprising a solubilizing agent, for example urea. Advantageously, the urea may be present at a concentration of at least 0.1M or at least 1M or at least 2.5M, or about 5M. An alternative solubilizing agent which may be used is guanidine, at a concentration of between 0.1M and 8M, preferably at least 1M or at least 2.5M. Prior to refolding, a reducing agent is preferably employed to ensure complete reduction of cysteine residues. Further denaturing agents such as DTT and guanidine may be used as necessary. Different denaturants and reducing agents may be used prior to the refolding step (e.g. urea, β-mercaptoethanol). Alternative redox couples may be used during refolding, such as a cystamine/cysteamine redox couple, DTT or β-mercaptoethanol/atmospheric oxygen, and cysteine in reduced and oxidized forms.

The monomeric TCR described herein may be labelled with a detectable label, for example a label which is suitable for diagnostic purposes. Thus, the invention provides a method for detecting MHC-peptide complexes which method comprises contacting the MHC-peptide complexes with a TCR in accordance with the invention which is specific for the MHC-peptide complex; and detecting binding of the TCR to the MHC-peptide complex. In tetrameric TCR formed using biotinylated heterodimers, fluorescent streptavidin (commercially available) can be used to provide a detectable label. A fluorescently labelled tetramer will be suitable for use in FACS analysis, for example to detect antigen presenting cells carrying the peptide for which the TCR is specific.

Another manner in which the soluble TCRs may be detected is by the use of TCR-specific antibodies, in particular monoclonal antibodies. There are many commercially available anti-TCR antibodies, such as βFI and αFI, which recognize the constant regions of the β and α chain, respectively.

The TCR may alternatively or additionally be linked to a therapeutic agent which may be for example a toxic moiety for example for use in cell killing, or an immunostimulating agent such as an interleukin or a cytokine. Thus, the invention provides a method for delivering a therapeutic agent to a target cell, which method comprises contacting potential target cells with a TCR in accordance with the invention under conditions to allow attachment of the TCR to the target cell, said TCR being specific for the MHC-peptide complexes and having the therapeutic agent associated therewith. In particular, the soluble TCR could be used to deliver therapeutic agents to the location of cells presenting a particular antigen. For instance, a toxin could be delivered to a tumor thereby helping to eradicate it. This would be useful in many situations and in particular against tumors because not all cells in the tumor present antigens and therefore not all tumor cells are detected by the immune system. With the soluble TCR, a compound could be delivered such that it would exercise its effect locally but not only on the cell it binds to. Thus, one particular strategy envisages anti-tumor molecules linked to T cell receptors specific for tumor antigens.

Many toxins could be employed for this use, for instance radioactive compounds, enzymes (perforin for example) or chemotherapeutic agents (cis-platin for example). To ensure that toxic effects are exercised in the desired location the toxin could be inside a liposome linked to streptavidin so that the compound is released slowly. This will prevent damaging effects during the transport in the body and ensure that the toxin has maximum effect after binding of the TCR to the relevant antigen presenting cells.

An alternative way to label or attach another moiety such as a toxic moiety to the soluble TCR is by including the label or other moiety in a mixed molecule multimer. An example of such a multimeric molecule is a tetramer containing three TCR molecules and one peroxidase molecule. This could be achieved by mixing the TCR and the enzyme at a molar ratio of 3:1 to generate tetrameric complexes and isolating the desired complex from any complexes not containing the correct ratio of molecules. Mixed molecules could contain any combination of molecules, provided that steric hindrance does not compromise or does not significantly compromise the desired function of the molecules. The positioning of the binding sites on the streptavidin molecule is suitable for mixed tetramers since steric hindrance is not likely to occur.

Preferably, the recombinant TCR chains according to the invention have a flexible linker located between the TCR domain and the dimerization peptide. Suitable flexible linkers include standard peptide linkers containing glycine, for example linkers containing glycine and serine. C-terminal truncations close to the cysteine residues forming the interchain disulfide bond are believed to be advantageous because the α and β chains are in close proximity through these residues in cellular TCRs. Therefore only relatively short linker sequences may be required to supply a nondistortive transition from the TCR chains to the heterodimerization domain. It is preferred that the linker sequences Pro-Gly-Gly or Gly-Gly are used. However, the linker sequence could be varied. For instance, the linker could be omitted completely, or reduced to a single residue, the preferred choice in this case being a single Glycine residue. Longer linker variations are also likely to be tolerated in the soluble TCR, provided that they could be protected from protease attack which would lead to segregation of the dimerization peptides from the extracellular domains of the TCR with ensuing loss of α-β chain stability.

The soluble TCR according to the invention is not necessarily α-β TCR. Molecules such as γ-δ, α-δ- and γ-β-TCR molecules, as well as TCR molecules (pre-TCR) containing invariant alpha chains which are only expressed early in development are also included. Pre-TCR specifies the cell lineage which will express α-β T cell receptor, as opposed to those cells which will express γ-β T cell receptor (for reviews, see Aifantis, Azogui et al. 1998; von Boehmer, Aifantis et al. 1998; Wurch, Biro et al. 1998). The Pre-TCR is expressed with the TCR β chain pairing with an invariant Pre-TCR α chain (Saint Ruf, Ungewiss et al. 1994; Wilson and MacDonald 1995) which appears to commit the cell to the α-β T cell lineage. The role of the Pre-TCR is therefore thought to be important during thymus development (Ramiro, Trigueros et al. 1996).

Standard modifications to the recombinant proteins according to the invention may be made as appropriate. These include, for example, altering an unpaired cysteine residue in the constant region of the β chain to avoid incorrect intrachain or interchain pairing.

The signal peptide may be omitted since it does not serve any purpose in the mature receptor or for its ligand binding ability, and may in fact prevent the TCR from being able to recognize ligand. In most cases, the cleavage site at which the signal peptide is removed from the mature TCR chains is predicted but not experimentally determined. Engineering the expressed TCR chains such that they are a few, i.e. up to about 10 for example, amino acids longer or shorter at the N-terminal end will have no significance for the functionality of the soluble TCR. Certain additions which are not present in the original protein sequence could be added. For example, a short tag sequence which can aid in purification of the TCR chains could be added provided that it does not interfere with the correct structure and folding of the antigen binding site of the TCR.

For expression in E. coli, a methionine residue may be engineered onto the N-terminal starting point of the predicted mature protein sequence in order to enable initiation of translation.

Far from all residues in the variable domains of TCR chains are essential for antigen specificity and functionality. Thus, a significant number of mutations can be introduced in this region without affecting antigen specificity and functionality.

By contrast, certain residues involved in forming contacts to the peptide antigen or the HLA heavy chain polypeptide, i.e. the residues constituting the CDR regions of the TCR chains, may be substituted for residues that would enhance the affinity of the TCR for the ligand. Such substitutions, given the low affinity of most TCRs for peptide-MHC ligands, could be useful for enhancing the specificity and functional potential of soluble TCRs. In the examples herein, the affinities of soluble TCRs for peptide-MHC ligands are determined. Such measurements can be used to assay the effects of mutations introduced in the TCR and thus also for the identification of TCRs containing substitutions which enhance the activity of the TCR.

Far from all residues in the constant domains of TCR chains are essential for antigen specificity and functionality. Thus, a significant number of mutations can be introduced in this region affecting antigen specificity. In Example 14 below, we have shown that two amino acid substitutions in the constant domain of a TCR β chain had no detectable consequences for the ability of the TCR to bind a HLA-peptide ligand.

The TCR β chain contains a cysteine residue which is unpaired in the cellular or native TCR. Mutation of this residue enhances the efficiency of in vitro refolding of soluble TCR. Substitutions of this cysteine residue for serine or alanine has a significant positive effect on refolding efficiencies in vitro. Similar positive effects, or even better effects, may be obtained with substitutions for other amino acids.

As mentioned previously, it is preferred that the cysteine residues forming the interchain disulfide bond in native TCR are not present so as to avoid refolding problems. However, since the alignment of these cysteine residues is the natural design in the TCR and also has been shown to be functional with this alignment for the c-jun and c-fos leucine zipper domains (O'Shea et al. 1989), these cysteine residues could be included provided that the TCR could be refolded.

Because the constant domains are not directly involved in contacts with the peptide-MHC ligands, the C-terminal truncation point may be altered substantially without loss of functionality. For instance, it should be possible to produce functional soluble TCRs excluding the entire constant domain. In principle, it would be simpler to express and fold soluble TCRs comprising only the variable regions or the variable regions and only a short fragment of the constant regions, because the polypeptides would be shorter. However, this strategy is not preferred. This is because the provision of additional stability of the α-β chain pairing through a heterodimerization domain would be complicated because the engineered C-termini of the two chains would be some distance apart, necessitating long linker sequences. The advantage of fusing heterodimerization domains just prior to the position of the cysteines forming the interchain disulfide bond, as is preferred, is that the α and β chains are held in close proximity in the cellular receptor. Therefore, fusion at this point is less likely to impose distortion on the TCR structure.

It is possible that functional soluble TCR could be produced with a larger fragment of the constant domains present than is preferred herein, i.e. the constant domains need not be truncated just prior to the cysteines forming the interchain disulfide bond. For instance, the entire constant domain except the transmembrane domain could be included. It would be advantageous in this case to mutate the cysteine residues forming the interchain disulfide bond in the cellular TCR.

In addition to aiding interchain stability through a heterodimerization domain, incorporation of cysteine residues which could form an interchain disulfide bond could be used. One possibility would be to truncate the α and β chains close to the cysteine residues forming the interchain disulfide bond without removing these so that normal disulfide bonding could take place. Another possibility would be to delete only the transmembrane domains of the α and β chains. If shorter fragments of the α and β chains were expressed, cysteine residues could be engineered in as substitutions at amino acid positions where the folding of the two chains would bring the residues in close proximity, suitable for disulfide bond formation.

Purification of the TCR may be achieved by many different means. Alternative modes of ion exchange may be employed or other modes of protein purification may be used such as gel filtration chromatography or affinity chromatography.

In the method of producing a recombinant TCR in accordance with the invention, folding efficiency may also be increased by the addition of certain other protein components, for example chaperone proteins, to the refolding mixture. Improved refolding has been achieved by passing protein through columns with immobilized mini-chaperones (Altamirano, Golbik et al. 1997; Altamirano, Garcia et al. 1999).

In addition to the methods described in the examples, alternative means of biotinylating the TCR may be possible. For example, chemical biotinylation may be used. Alternative biotinylation tags may be used, although certain amino acids in the biotin tag sequence are essential (Schatz et al. 1993). The mixture used for biotinylation may also be varied. The enzyme requires Mg-ATP and low ionic strength, although both of these conditions may be varied, e.g. it may be possible to use a higher ionic strength and a longer reaction time. It may be possible to use a molecule other than avidin or streptavidin to form multimers of the TCR. Any molecule which binds biotin in a multivalent manner would be suitable. Alternatively, an entirely different linkage could be devised (such as poly-histidine tag to chelated nickel ion (Quiagen Product Guide 1999, Chapter 3 “Protein Expression, Purification, Detection and Assay” p. 35-37)). Preferably, the tag is located towards the C-terminus of the protein so as to minimize the amount of steric hindrance in the interaction with potential peptide-MHC complexes.

Examples of suitable MHC-peptide targets for the TCR according to the invention include, but are not limited to, viral epitopes such as HTLV-1 epitopes (e.g. the Tax peptide restricted by HLA-A2; HTLV-1 is associated with leukaemia), HIV epitopes, EBV epitopes, CMV epitopes; melanoma epitopes and other cancer-specific epitopes; and epitopes associated with autoimmune disorders, such as rheumatoid arthritis.

A multitude of disease treatments can potentially be enhanced by localizing the drug through the specificity of soluble TCRs.

Viral diseases for which drugs exist, e.g. HIV, SIV, EBV, CMV, would benefit from the drug being released in the near vicinity of infected cells. For cancer, the localization in the vicinity of tumors or metastases would enhance the effect of toxins or immunostimulants. In autoimmune diseases, immunosuppressive drugs could be released slowly, having more local effect over a longer time-span while minimally affecting the overall immuno-capacity of the subject. In the prevention of graft rejection, the effect of immunosuppressive drugs could be optimized in the same way. For vaccine delivery, the vaccine antigen could be localized in the vicinity of antigen presenting cells, thus enhancing the efficacy of the antigen. The method can also be applied for imaging purposes.

Preferred features of each aspect of the invention are as for each of the other aspects mutatis mutandis. The prior art documents mentioned herein are incorporated to the fullest extent permitted by law.

The invention is further described in the following examples, which do not limit the scope of the invention in any way.

Reference is made in the following to the accompanying drawings in which:

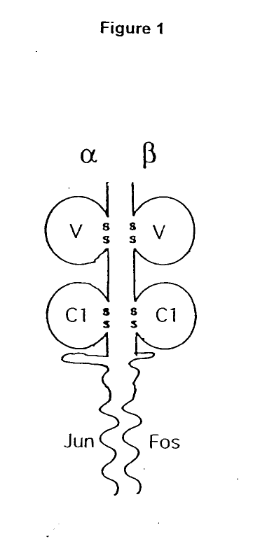

FIG. 1 is a schematic view of a T-cell Receptor-leucine zipper fusion protein. Each chain consists of two immunoglobulin superfamily domains, one variable (V) and one constant (C). The constant domains are truncated immediately N-terminal of the interchain cysteine residues, and fused to a leucine zipper heterodimerization motif from c-Jun (α) or c-Fos (β) of around 40 amino acids at the C-terminal via a short linker. The α-Jun and β-Fos fusion proteins each contain two intrachain disulfide bonds and pair solely by non-covalent contacts. The α chain is shorter than the β chain due to a smaller constant domain.

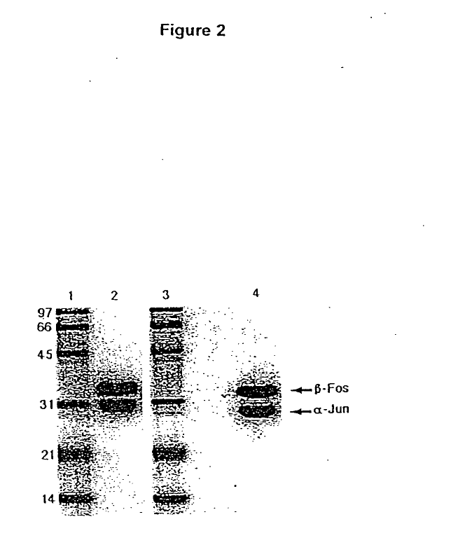

FIG. 2 is a photograph of a reducing/non-reducing gel analysis of heterodimeric JM22zip receptor. Identical samples of purified TCR-zipper were loaded onto a 15% acrylamide SDS gel, either under reducing conditions (lane 2) or non-reducing conditions (lane 4). Marker proteins are shown in lanes 1 and 3. Molecular weights are shown in kilodaltons. Under both sets of conditions, the non-covalently associated heterodimer is dissociated into α and β chains. In lane 4, each chain runs with a higher mobility and as a single band, indicating a single species of intra-chain disulfide bonding is present. This is compatible with correct disulfide bond formation.

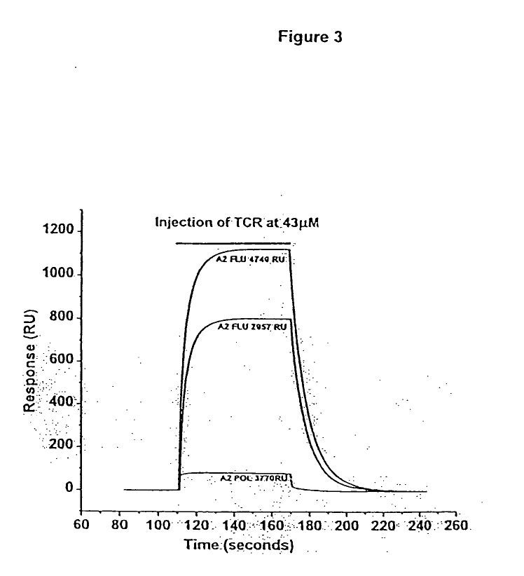

FIG. 3 is a graph showing the specific binding of JM22zip TCR to HLA-A2 Flu matrix (M58-66) complexes. HLA-A2 complexes, refolded around single peptides and biotinylated on β2-microglobulin have been immobilized onto three streptavidin-coated flow cells: 3770 Resonance Units (RU) of HLA-A2 POL control onto flow cell (FC) 3, and two different levels of HLA-A2 M58-66 FLU (2970 RU on FC1 and 4960 RU on FC2). JM22zip has been injected in the soluble phase sequentially over all three flow cells at a concentration of 43 μM for 60 seconds. During the injection, an above-background increase in the response of both HLA-A2 FLU-coated flow cells is seen, with approximately 1000 RU and 700 RU of specific binding of JM22zip to flow cells 1 and 2 respectively.

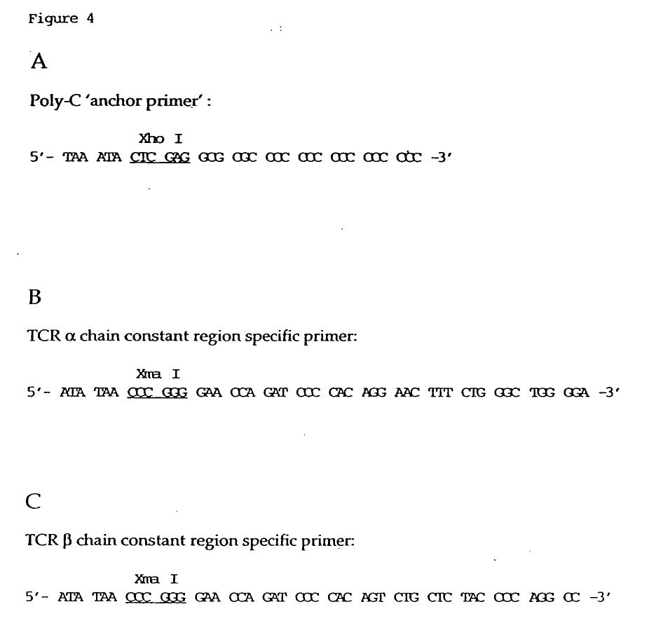

FIG. 4 shows the sequences of synthetic DNA primers used for “anchor amplification” of TCR genes. Recognition sites for DNA restriction enzymes used for cloning are underlined. A: poly-C “anchor primer” (SEQ ID NO. 1). B: TCR α chain constant region specific primer (SEQ ID NO. 2). C: TCR β chain constant region specific primer (SEQ ID NO. 3).

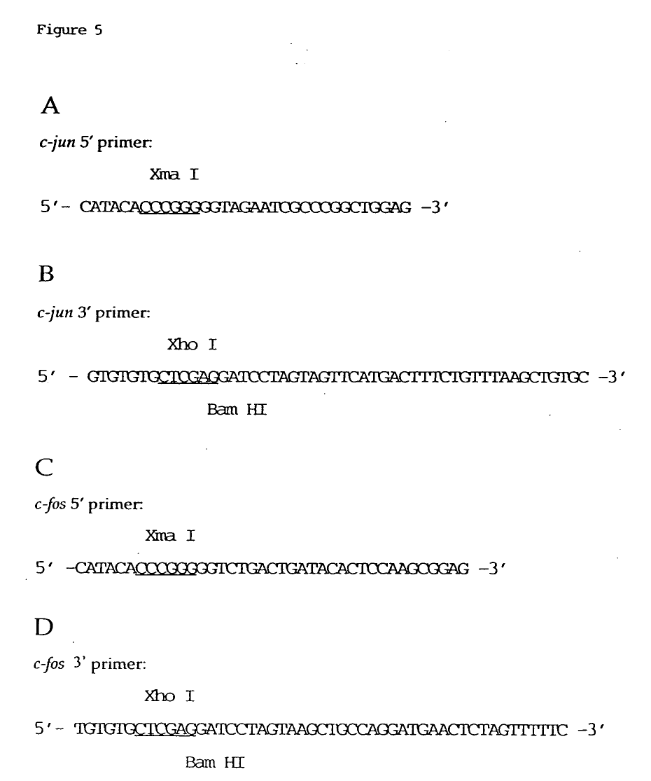

FIG. 5 shows the sequences of synthetic DNA primers used for PCR amplification of DNA fragments encoding the 40 amino acid coiled-coil (“leucine zipper”) regions of c-jun and c-fos. Recognition sites for DNA restriction enzymes used for cloning are underlined. A: c-jun 5′ primer (SEQ ID NO. 4). B: c-jun 3′ primer (SEQ ID NO. 5). C: c-fos 5′ primer (SEQ ID NO. 6). D: c-fos 3′ primer (SEQ ID NO. 7).

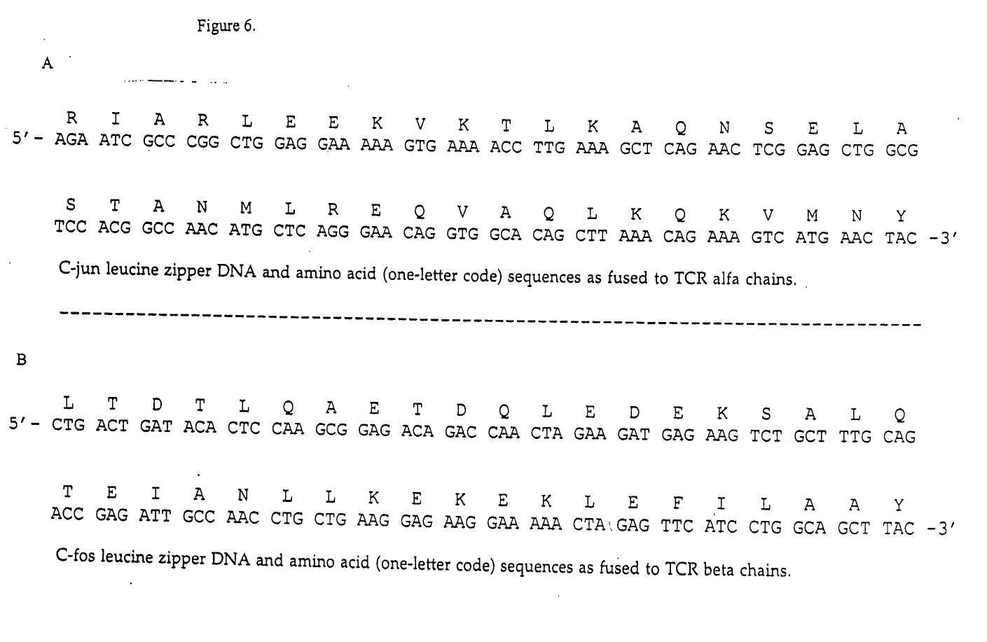

FIG. 6 shows the respective DNA and amino acid (one letter code) sequences of c-fos, and c-jun fragments as fused to TCRs (inserts in pBJ107 and pBJ108). A: c-jun leucine zipper as fused to TCR α chains (SEQ ID NO. 9 (DNA sequence) and SEQ ID NO. 10 (amino acid sequence)). B: c-fos leucine zipper as fused to TCR β chains (SEQ ID NO. 8 (DNA sequence)) and SEQ ID NO. 11 (amino acid sequence)).

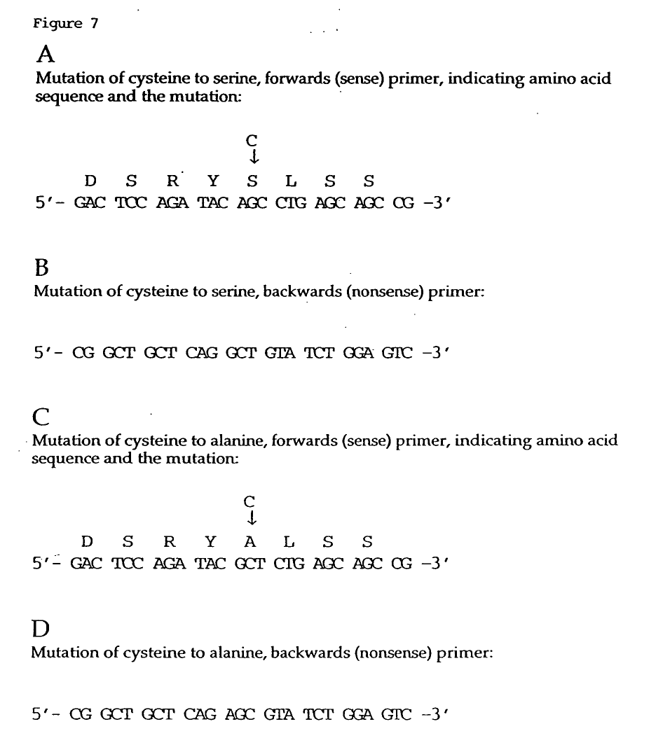

FIG. 7 shows the sequences of the synthetic DNA primers used for mutating the unpaired cysteine residue in TCR β chains. The primers were designed for used with the “QUIKCHANGE™” method for mutagenesis (Stratagene). A: Mutation of cysteine to serine, forwards (sense) primer, indicating amino acid sequence (SEQ ID NO. 12 (DNA sequence)) and SEQ ID NO. 13 (amino acid sequence)) and the mutation. B: mutation of cysteine to serine, backwards (nonsense) primer (SEQ ID NO. 14). C: mutation of cysteine to alanine, forwards (sense) primer, indicating amino acid sequence (SEQ ID NO. 16 (amino acid sequence)) and SEQ ID NO. 15 (DNA sequence)) and the mutation. D: mutation of cysteine to alanine, backwards (nonsense) primer (SEQ ID NO. 17).

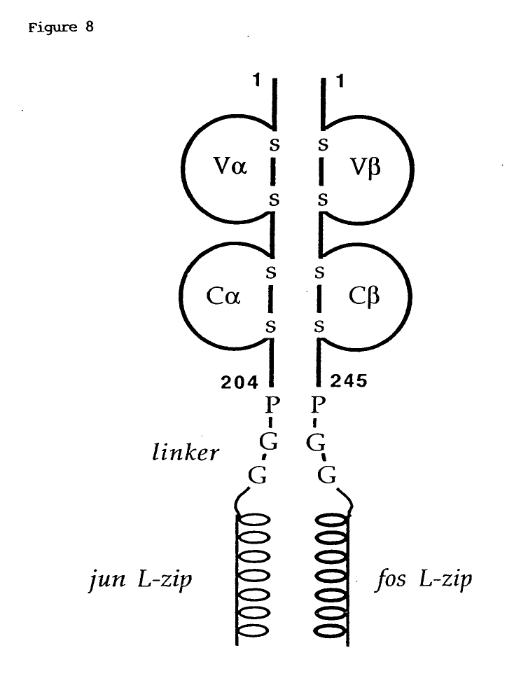

FIG. 8 is a schematic representation of a TCR-zipper fusion protein. The four immunoglobulin domains are indicated as domes, with the intrachain disulfide bridges between matching pairs of cysteine residues shown. The numbers indicate amino acid positions in the mature T-cell receptor chains; due to slight variation in chain length after recombination, the lengths of the chains can vary slightly between different TCRs. The residues introduced in the linker sequences are indicated in the one-letter code.

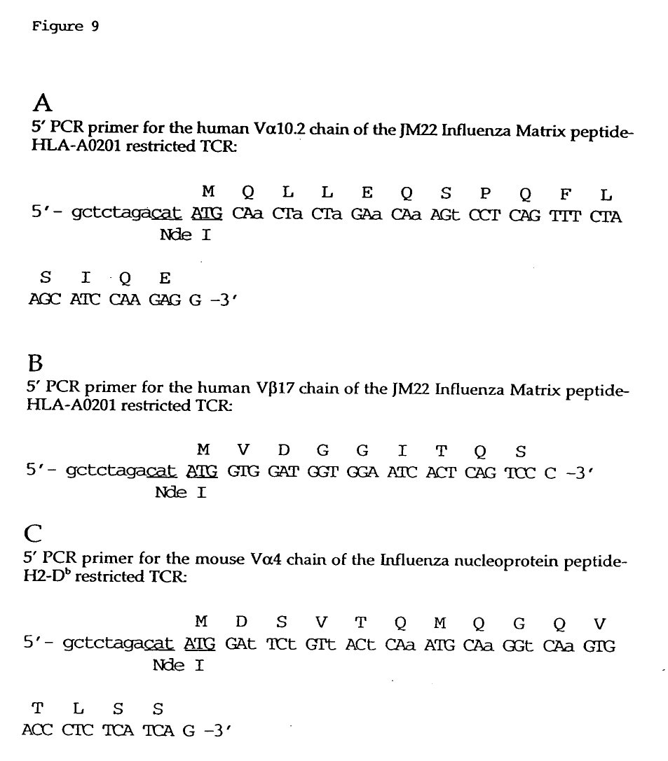

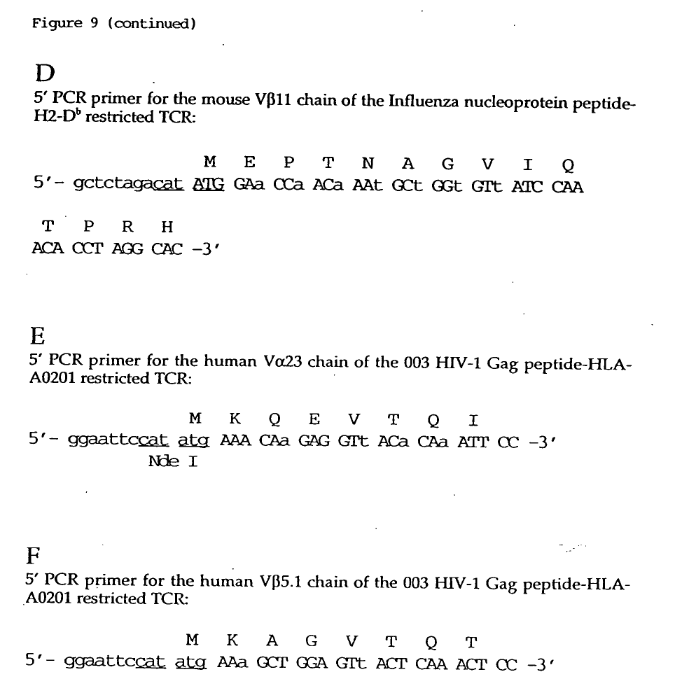

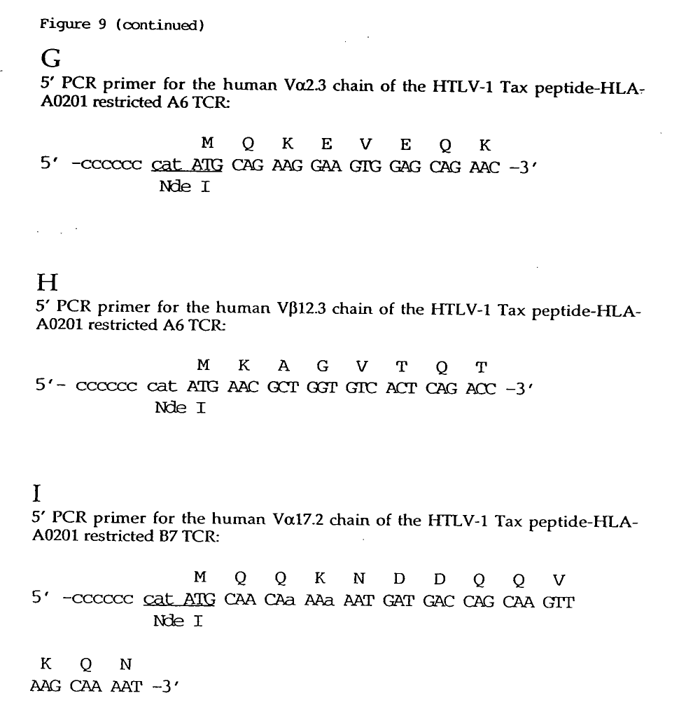

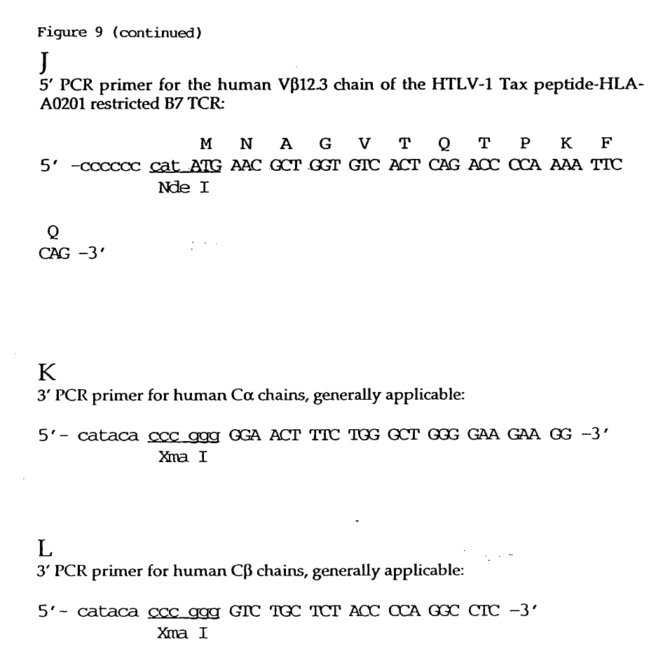

FIG. 9 shows the sequences of the synthetic DNA primers used for PCR amplification of TCR α and β chains. Recognition sites for DNA restriction enzymes are underlined and the amino acid sequences corresponding to the respective TCR chains are indicated over the forward primer sequences. Silent DNA mutations relative to the TCR gene sequences and other DNA sequences which do not correspond to the TCR genes are shown in lower case letters. A: 5′ PCR primer for the human Vα10.2 chain of the JM22 Influenza Matrix virus peptide-HLA-A0201 restricted TCR (SEQ ID NO. 18 and SEQ ID NO. 19). B: 5′ PCR primer for the human Vβ17 chain of the JM22 Influenza Matrix virus peptide-HLA-A0201 restricted TCR (SEQ ID NO. 20 and SEQ ID NO. 21). C: 5′ PCR primer for the mouse Vα4 chain of the Influenza nucleoprotein peptide-H2-D^{b} restricted TCR (SEQ ID NO. 22 and SEQ ID NO. 23). D: 5′ PCR primer for the mouse VβI1 chain of the Influenza nucleoprotein peptide-H2-D^{b} restricted TCR (SEQ ID NO. 24 and SEQ ID NO. 25). E: 5′ PCR primer of the human Vα23 chain of the 003 HIV-1 Gag peptide-HLA-A0201 restricted TCR (SEQ ID NO. 26 and SEQ ID NO. 27). F: 5′ PCR primer of the human Vβ5.1 chain of the 003 HIV-1 Gag peptide-HLA-A0201 restricted TCR (SEQ ID NO. 28 and SEQ ID NO. 29). G: 5′ PCR primer of the human Vα2.3 chain of the HTLV-1 Tax peptide-HLA-A0201 restricted A6 TCR (SEQ ID NO. 30 and SEQ ID NO. 31). H: 5′ PCR primer of the human Vβ12.3 chain of the HTLV-1 Tax peptide-HLA-A0201 restricted A6 TCR (SEQ ID NO. 32 and SEQ ID NO. 33). 1:5′ PCR primer of the human VαI7.2 chain of the HTLV-1 Tax peptide-HLA-A0201 restricted B7 TCR (SEQ ID NO. 34 and SEQ ID NO. 35). J: 5′ PCR primer of the human VβI2.3 chain of the HTLV-1 Tax peptide-HLA-A0201 restricted B7 TCR (SEQ ID NO. 36 and SEQ ID NO. 37). K: 3′ PCR primer for human Ca chains, generally applicable (SEQ ID NO. 38). L: 3′ PCR primer for human Cβ chains, generally applicable (SEQ ID NO. 39).

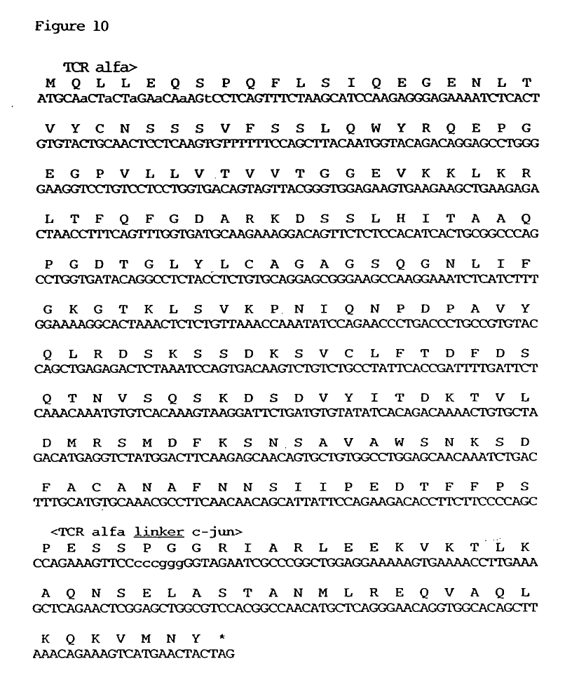

FIG. 10 shows the predicted protein sequence (one letter code, top) (SEQ ID NO. 41) and DNA sequence (bottom) (SEQ ID NO. 40) of the soluble HLA-A2/flu matrix restricted TCR α chain from JM22, as fused to the “leucine zipper” domain of c-jun. Mutations introduced into the 5′ end of the DNA sequence to enhance expression of the gene in E. coli are indicated in small letters, as is the linker sequence between the TCR and c-jun sequences.

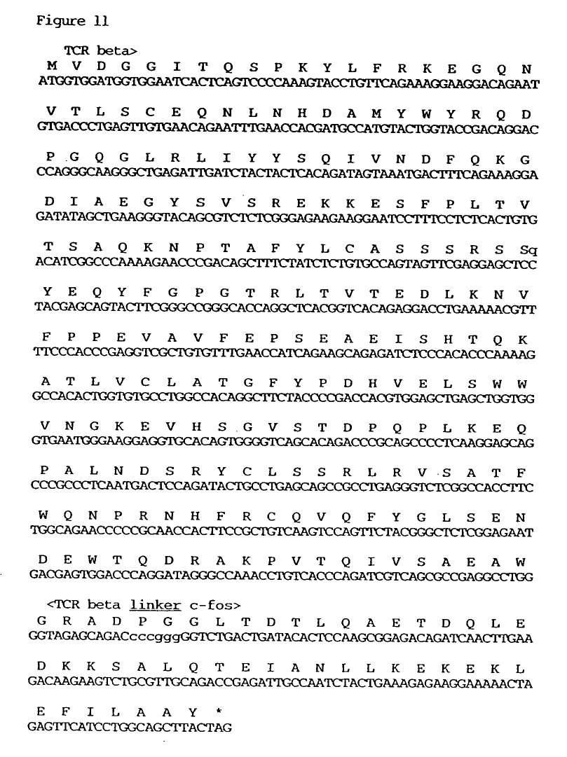

FIG. 11 shows the predicted protein sequence (one letter code, top) (SEQ ID NO. 43) and DNA sequence (bottom) (SEQ ID NO. 42) of the soluble HLA-A2/flu matrix restricted TCR β chain from JM22, as fused to the “leucine zipper” domain of c-fos. The linker sequence between the TCR and c-fos sequences is indicated in small letters.

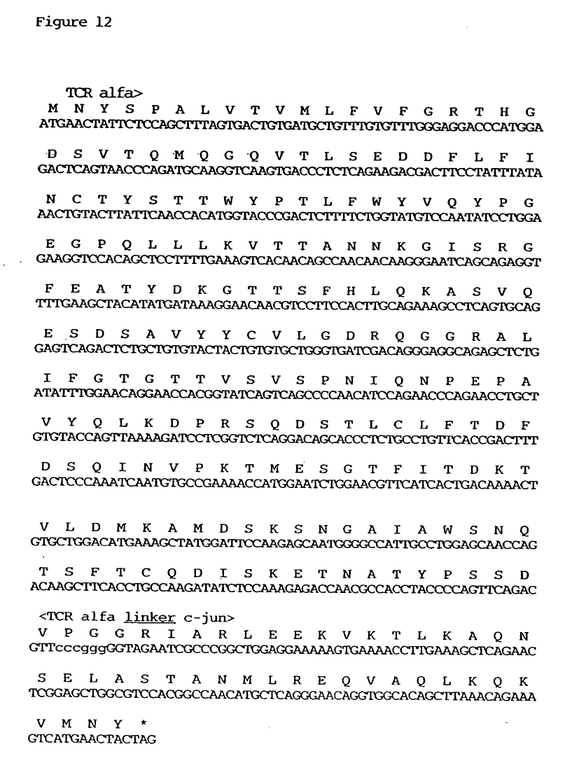

FIG. 12 shows the predicted protein sequence (one letter code, top) (SEQ ID NO. 45) and DNA sequence (bottom) (SEQ ID NO. 44) of the soluble H2-D^{b}/Influenza virus nucleoprotein restricted TCR α chain from murine F5 receptor, as fused to the “leucine zipper” domain of c-jun. Mutations introduced into the 5′ end of the DNA sequence to enhance expression of the gene in E. coli are indicated in small letters, as is the linker sequence between the TCR and c-jun sequences.

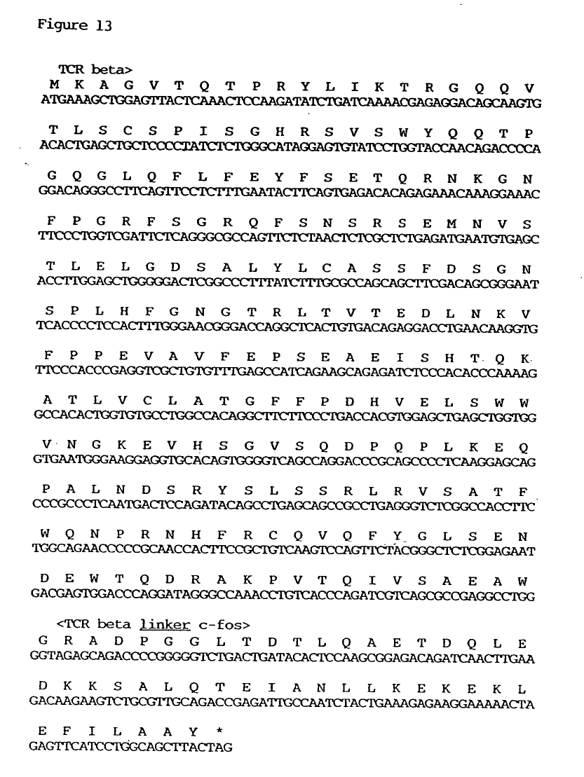

FIG. 13 shows the predicted protein sequence (one letter code, top) (SEQ ID NO. 47) and DNA sequence (bottom) (SEQ ID NO. 46) of the soluble H2-D^{b}/Influenza virus nucleoprotein restricted TCR β chain from murine F5 receptor, as fused to the “leucine zipper” domain of c-fos. The linker sequence between the TCR and c-fos sequences is indicated in small letters.

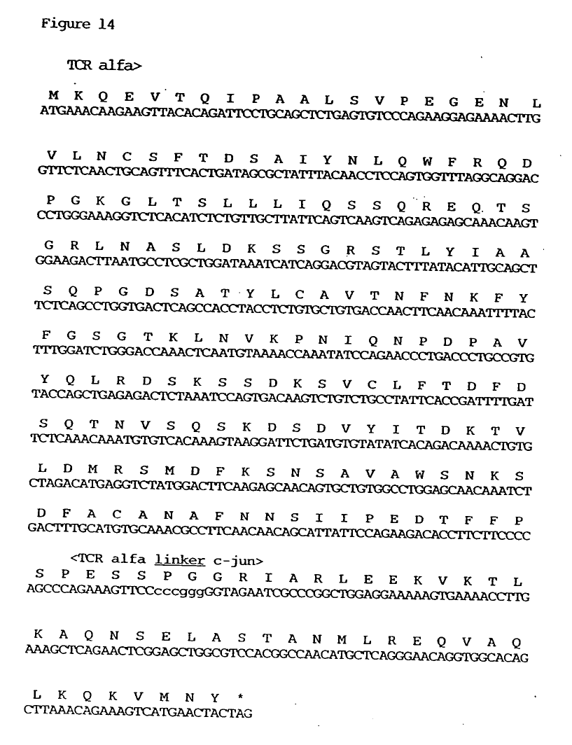

FIG. 14 shows the predicted protein sequence (one letter code, top) (SEQ ID NO. 49) and DNA sequence (bottom) (SEQ ID NO. 48) of the soluble HLA-A2/HIV-1 Gag restricted TCR β chain from patient 003, as fused to the “leucine zipper” domain of c-jun. Mutations introduced into the 5′ end of the DNA sequence to enhance expression of the gene in E. coli are indicated in small letters, as is the linker sequence between the TCR and c-jun sequences.

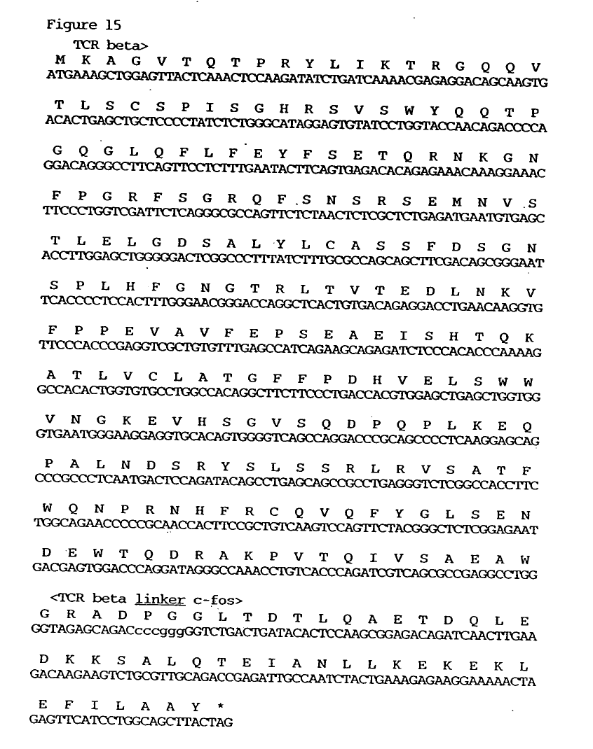

FIG. 15 shows the predicted protein sequence (one letter code, top) (SEQ ID NO. 51) and DNA sequence (bottom) (SEQ ID NO. 50) of the soluble HLA-A2/HIV-1 Gag restricted TCR β chain from patient 003, as fused to the “leucine zipper” domain of c-fos. The linker sequence between the TCR and c-fos sequences is indicated in small letters.

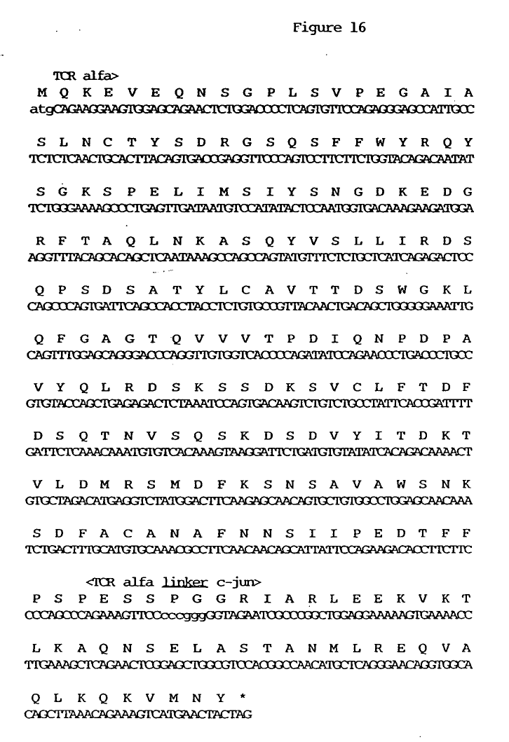

FIG. 16 shows the predicted protein sequence (one letter code, top) (SEQ ID NO. 53) and DNA sequence (bottom) (SEQ ID NO. 52) of the soluble HTLV-1 Tax/HLA-A2 restricted TCR α chain clone A6 (Garboczi, Utz et al. 1996; Garboczi, Ghosh et al. 1996), as fused to the “leucine zipper” domain of c-jun. Mutations introduced into the 5′ end of the DNA sequence to enhance expression of the gene in E. coli are indicated in small letters, as is the linker sequence between the TCR and c-jun sequences.

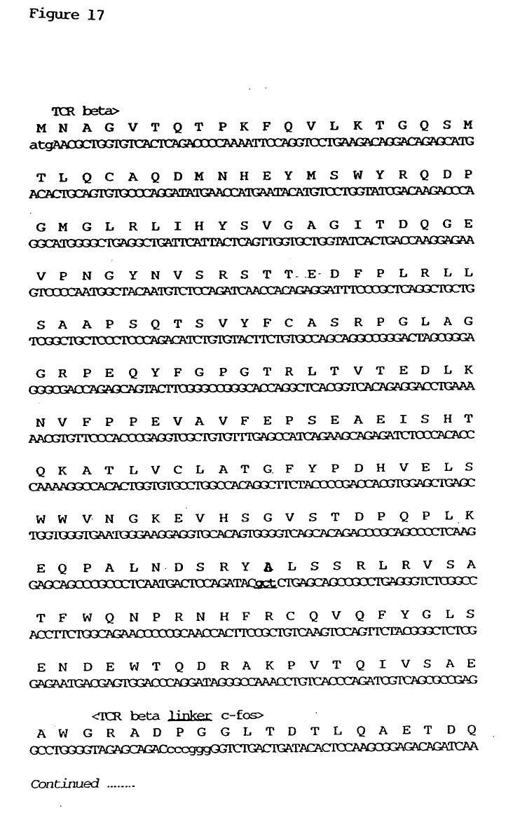

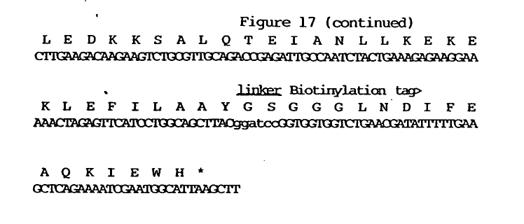

FIG. 17 shows the predicted protein sequence (one letter code, top) (SEQ ID NO. 55) and DNA sequence (bottom) (SEQ ID NO. 54) of the soluble HTLV-1 Tax/HLA-A2 restricted TCR β chain from clone A6 (Garboczi, Utz et al. 1996; Garboozi, Ghosh et al. 1996), as fused to the “leucine zipper” domain of c-fos and the biotinylation tag which acts as a substitute for BirA (Barker and Campbell, 1981; Barker and Campbell, 1981; Howard, Shaw et al., 1985; Schatz, 1993; O'Callaghan, Byford, 1999). The linker sequence between the TCR and c-fos sequences is indicated in small letters. Mutation of the DNA sequence which substitutes a cysteine residue for an alanine residue is indicated in bold and underlined.

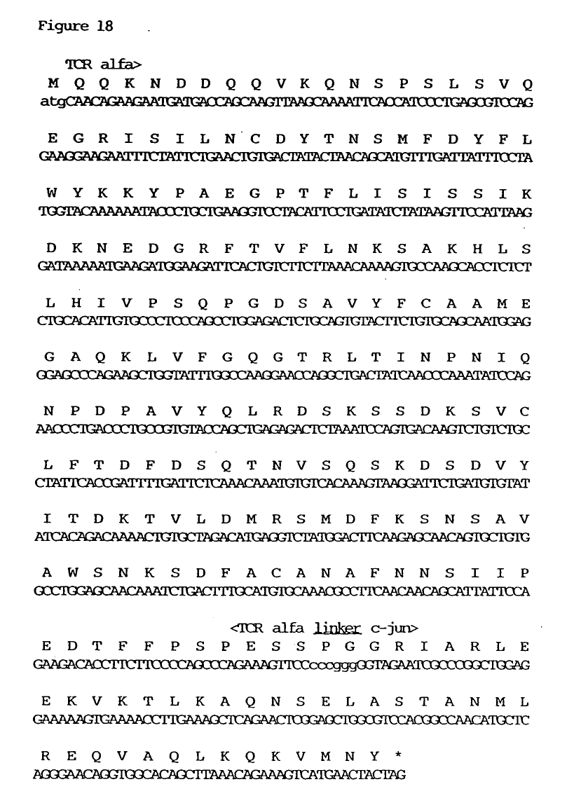

FIG. 18 shows the predicted protein sequence (one letter code, top) (SEQ ID NO. 57) and DNA sequence (bottom) (SEQ ID NO. 56) of the soluble HTLV-1 Tax/HLA-A2 restricted TCR α chain from clone M10B7/D3 (Ding et al. 1998), as fused to the “leucine zipper” domain of c-jun. The linker sequence between the TCR and c-jun sequences is indicated in small letters.

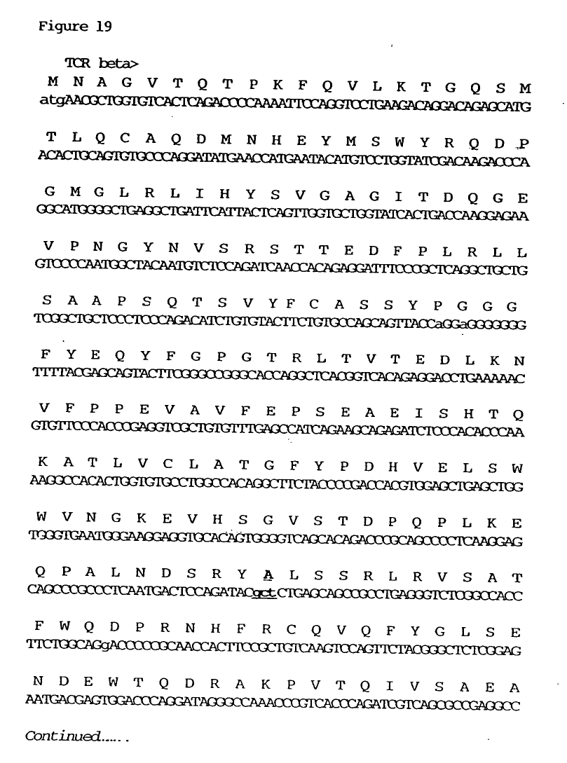

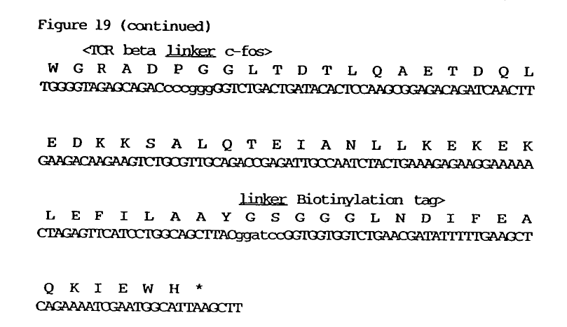

FIG. 19 shows the predicted protein sequence (one letter code, top) (SEQ ID NO. 59) and DNA sequence (bottom) (SEQ ID NO. 58) of the soluble HTLV-1 Tax/HLA-A2 restricted TCR β chain from clone M10B7/D3 (Ding et al. 1998), as fused to the “leucine zipper” domain of c-fos and the biotinylation tag which acts as a substitute for BirA. The linker sequence between the TCR and c-fos sequences is indicated in small letters. Mutation of the DNA sequence which substitutes an alanine for a cysteine residue is indicated in bold and underlined. Two silent mutations (P-G codons) introduced for cloning purposes and to remove a XmaI restriction site are also indicated in small letters.

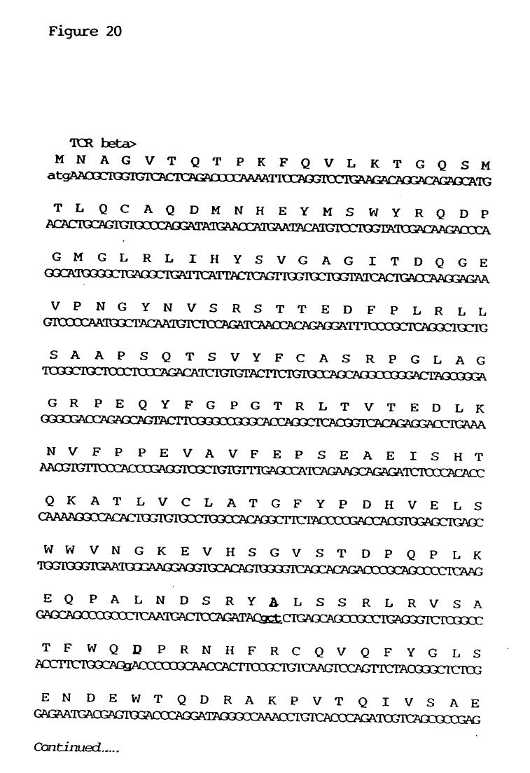

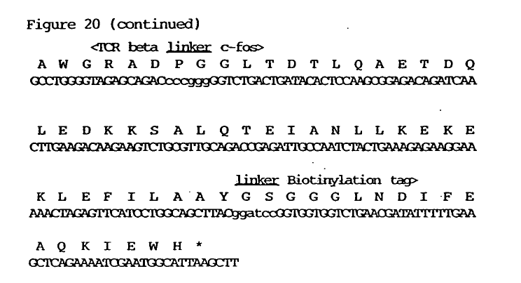

FIG. 20 shows the predicted protein sequence (one letter code, top) (SEQ ID NO. 61) and DNA sequence (bottom) (SEQ ID NO. 60) of mutated soluble HTLV-1 Tax/HLA-A2 restricted TCR β chain from clone A6 (Garboczi, Utz et al. 1996; Garboczi, Ghosh et al. 1996), as fused to the “leucine zipper” domain of c-fos and the biotinylation tag which acts as a substitute for BirA (Barker and Campbell, 1981; Barker and Campbell, 1981; Howard, Shaw, 1985; Schatz, 1993; O'Callaghan, Byford, 1999). The linker sequence between the TCR and c-fos sequences is indicated in small letters. Mutation of the DNA sequence which substitutes a cysteine residue for an alanine residue is indicated in bold and underlined. Also indicated in bold and underlined is a substitution of an asparagine residue for an aspartic acid, a mutation in the constant region which had no detectable functional effect on the soluble TCR.

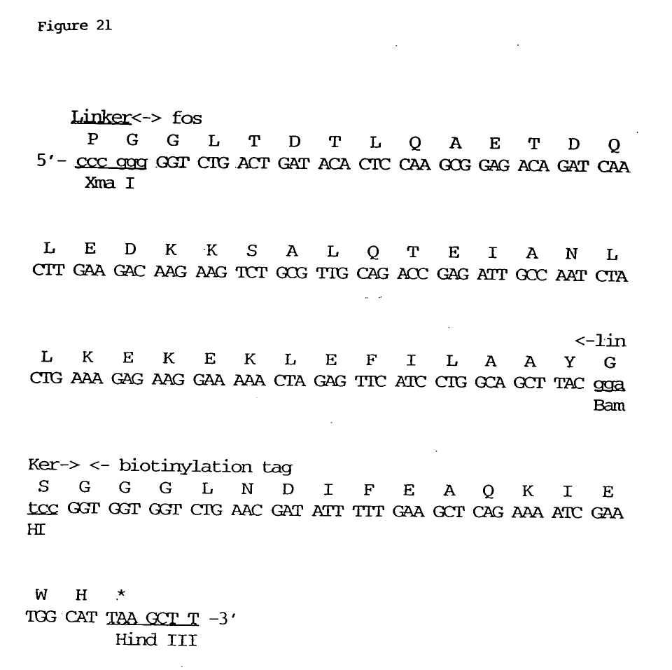

FIG. 21 shows the predicted protein sequence (one letter code, top) (SEQ ID NO. 63) and DNA sequence (bottom) (SEQ ID NO. 62) of the c-fos-biotinylation fusion partner used for TCR β chains. Recognition sites for DNA restriction enzymes are underlined and the borders of the two fusion domains are indicated. Linker sequences are shown in lower case letters.

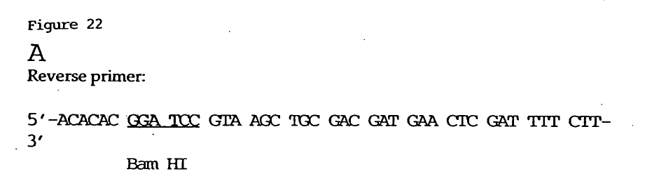

FIG. 22 shows the sequence of a synthetic DNA primer (SEQ ID NO. 64) used for PCR amplification of the Vβ3-c-fos leucine zipper fragment of the human JM22 Influenza Matrix peptide-HLA-A0201.

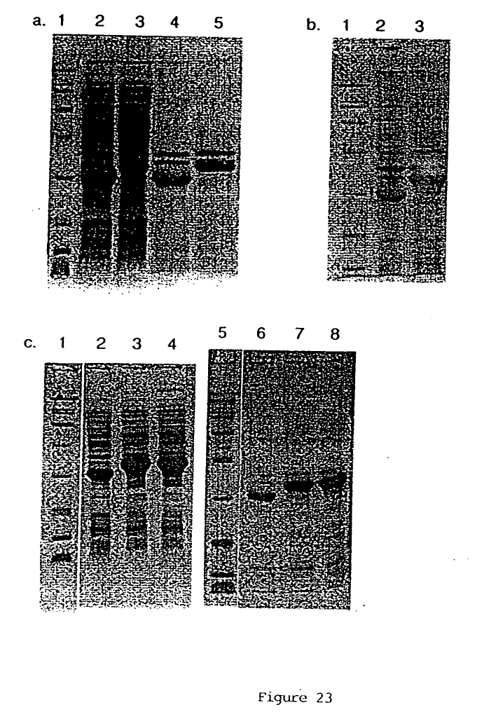

FIG. 23 is a set of photographs of gels. a. Preparation of denatured protein for the TCR specific for the 003 HIV gag peptide —HLA-A2 complex analyzed by SDS-PAGE. Lane 1: broad-range molecular weight markers (Bio-Rad), lanes 2 & 3: bacteria after induction of protein expression with 0.5 mM IPTG, lanes 4 & 5: purified inclusion bodies solubilised in 6M guanidine buffer. b. Preparation of denatured protein for the biotin-tagged TCR specific for the influenza matrix peptide—HLA-A2 complex analyzed by SDS-PAGE. Lane 1: broad-range molecular weight markers (Bio-Rad), lanes 2 & 3: α- & β-chain purified inclusion bodies solubilized in 6M guanidine buffer. c. Preparation of denatured protein for the biotin-tagged TCR specific for the HTLV tax peptide—HLA-A2 complex analyzed by SDS-PAGE. Lanes 1 & 5: broad-range molecular weight markers (Bio-Rad), lanes 2, 3 & 4: α-, β- & mutant β-chain expression in bacteria after induction of protein expression with 0.5 mM IPTG, lanes 6, 7 & 8: α-, β- & mutant β-chain purified inclusion bodies solubilized in 6M guanidine buffer.

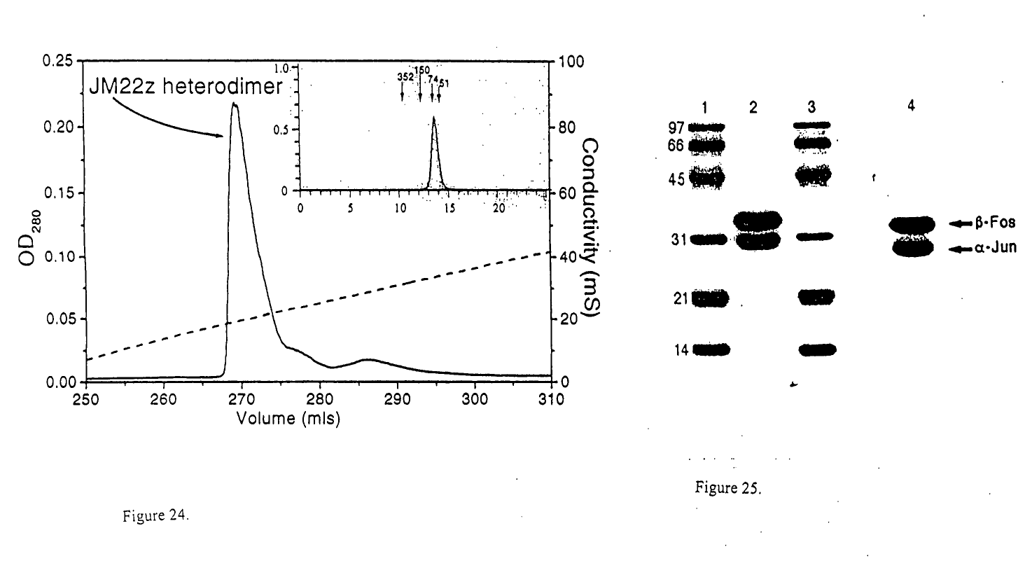

FIG. 24 is a chromatogram showing the elution of the JM22z heterodimer from a POROS™ 10HQ anion exchange column. Dashed line shows the conductivity which is indicative of a sodium chloride concentration, the solid line shows optical density at 280 nm which is indicative of protein concentration of the eluate. Peak protein containing fractions were pooled for further analysis. Insert shows a chromatogram of elution of purified JM22z from a SUPERDEX™ 200 HR column. Arrows indicate the calibration of the column with proteins of known molecular weight. By comparison with these proteins, the refolded JM22z protein has a molecular weight of approximately 74 kDA which is compatible with a heterodimeric protein.

FIG. 25 is a photograph showing an SDS-polyacrylamide gel electrophoresis (Coomassie-stained) of the purified JM22z protein. Lanes 1 & 3: standard proteins of known molecular weight (as indicated), lane 2: JM22z protein treated with SDS-sample buffer containing reducing agent (DTT) prior to sample loading, lane 4: JM22z protein treated with SDS-sample buffer in the absence of reducing agents.

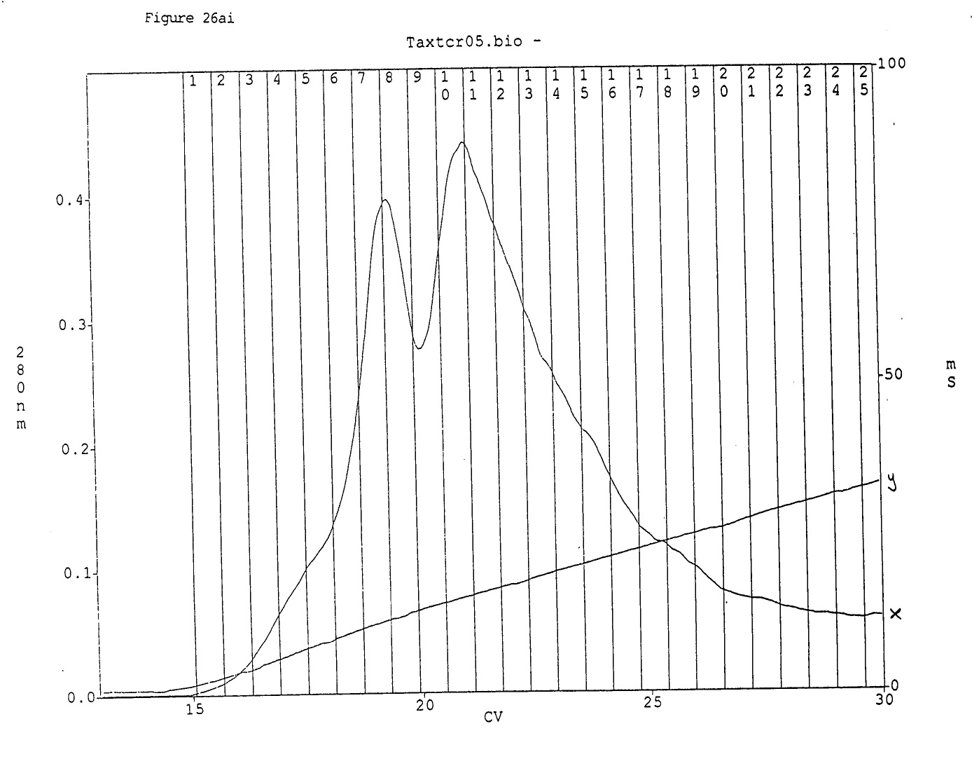

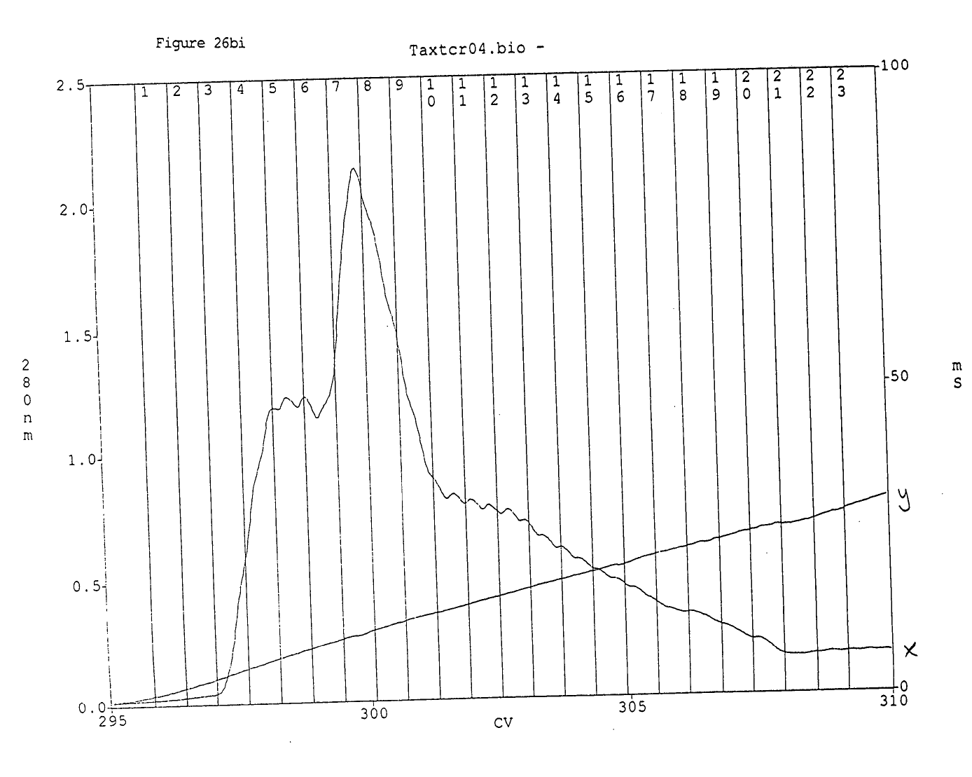

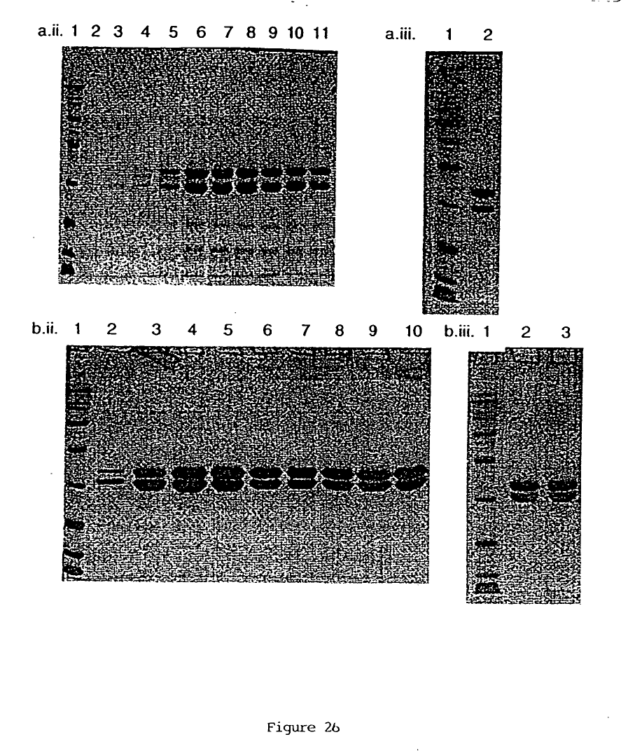

FIG. 26. a. Purification of the refolded biotin-tagged TCR specific for the influenza matrix peptide—HLA-A2 complex. i. Chromatogram of the elution of the protein from a POROS™ 10HQ column. Line x indicates absorbance at 280 nm and line y indicates conductivity (a measure of sodium chloride gradient used to elute the protein). Fraction numbers are indicated by the vertical lines. ii. SDS-PAGE of the fractions eluting off the column as in i. Lane 1 contains broad-range molecular weight markers (Bio-Rad) and lanes 2-13 contain 5 μl of fractions 6-15 respectively. iii. SDS-PAGE analysis of pooled fractions from i. containing biotin-tagged flu-TCR. Lane 1: broad-range molecular weight markers (Bio-Rad), lane 2: biotin-tagged flu-TCR protein. b. Purification of the refolded biotin-tagged TCR specific for the HTLV-tax peptide —HLA-A2 complex. i. Chromatogram of the elution of the protein from a POROS™ 10HQ column. Line x indicates absorbance at 280 nm and line y indicates conductivity (a measure of sodium chloride gradient used to elute the protein). Fraction numbers are indicated in by the vertical lines. ii. SDS-PAGE of the fractions eluting off the column as in i. Lane 1 contains broad-range molecular weight markers (Bio-Rad) and lanes 2-10 contain 5 μl of fractions 3-11 respectively. iii. SDS-PAGE analysis of pooled fractions from i. of biotin-tagged tax-TCR. Lane 1: broad-range molecular weight markers (Bio-Rad), lane 2: biotin-tagged tax-TCR protein, lane 3: mutant biotin-tagged tax-TCR protein.

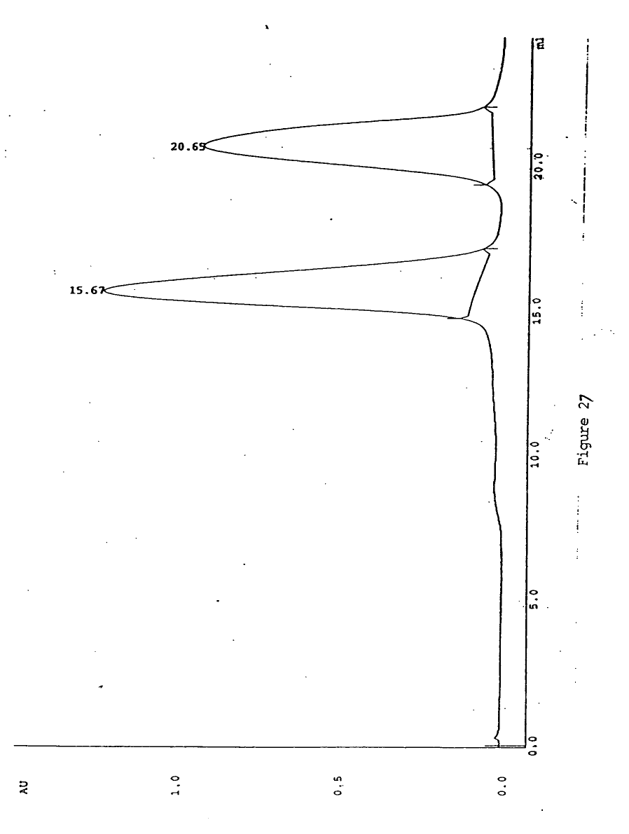

FIG. 27 is a chromatogram showing elution of biotin-tagged soluble TCR after biotinylation with BirA enzyme from a SUPERDEX™ 200 HR column equilibrated in PBS. The biotinylated TCR elutes at around 15-16 minutes and the free biotin elutes at around 21 minutes. Fractions containing biotinylated soluble TCR are pooled for future use.

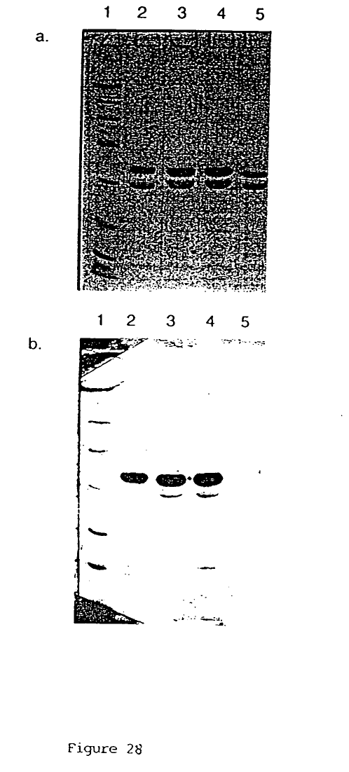

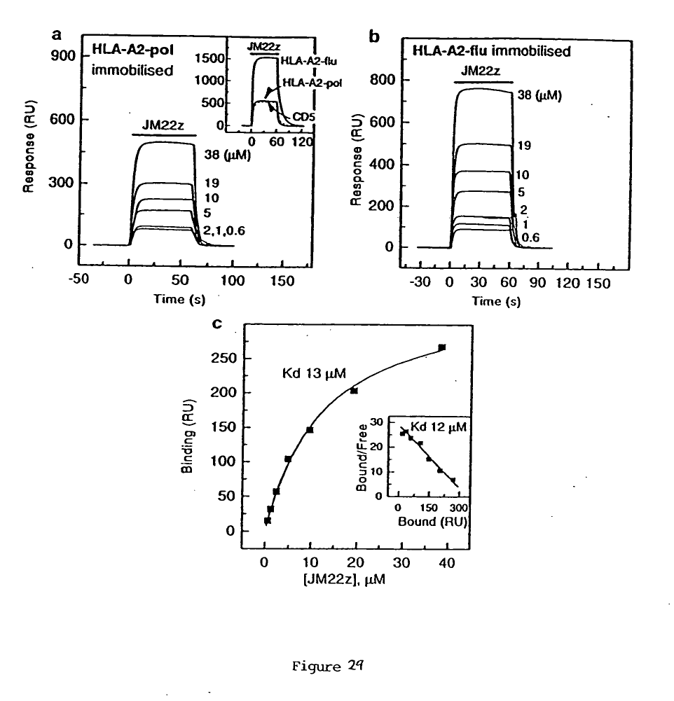

FIG. 28 is a set of photographs of gels. Assessment of biotinylation of the biotinylated TCRs. a. SDS-PAGE of refolded TCRs and inclusion body preparations. Lane 1: broad-range molecular weight markers (Bio-Rad), lane 2: Biotinylated flu-TCR, lane 3: Biotinylated tax-TCR, lane 4: Biotinylated mutant tax-TCR, lane 5: HIV gag-TCR, (not biotin-tagged); b. Western blot of a gel identical to a. except that the broad-range markers were biotin labelled (Bio-Rad). Staining was with avidin-HRP conjugate to show biotinylated proteins and visualization was with Opti-4CN (Bio-Rad).

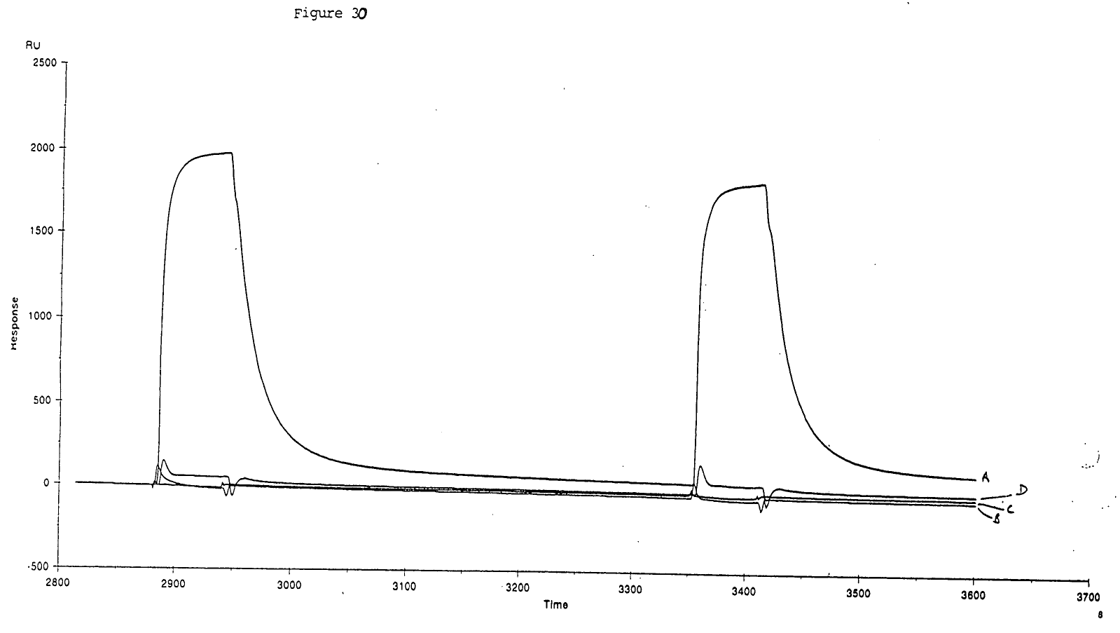

FIG. 29 illustrates JM22z binding to different HLA-A2-peptide complexes. (a inset). The specificity of the interaction between JM22z and HLA-A2-flu is demonstrated by comparing the SPR response from passing the TCR over a flow cell coated with 1900 RU of HLA-A2-flu to the responses from passing the TCR over two other flow cells one coated with 4200 RU of HLA-A2-pol, the other coated with 4300 RU of CD5. Background responses at different JM22z concentrations were measured on 1700 RU of HLA-A2-pol (a). The background value was subtracted from the specific response measured on 1900 RU of HLA-A2-flu (b) and plotted against concentration (c). The Kd of 13 μM, estimated by non-linear curve fitting was in accordance with the Kd of 12 μM calculated on basis of a Scatchard plot of the same data.

FIG. 30 is a graph showing the result of Biacore 2000™ analysis of wild-type and mutant soluble biotinylated tax TCR. 5 μl of wild-type tax TCR at a concentration of 2.2 mg/ml and then mutant tax TCR at a concentration of 2.4 mg/ml was flowed over four flow cells with the following proteins attached to the surface: A: tax-pMHC complex, B/C: flu-pMHC complex, D: OX68 control protein. Both wild-type and mutant proteins bind similarly to the specific pMHC complex.

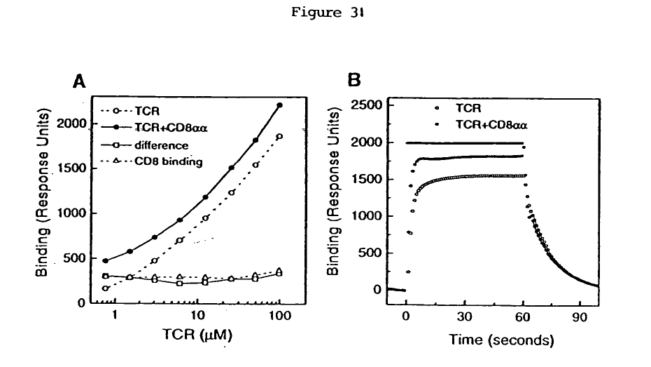

FIG. 31 shows the effect of soluble CD8aa binding on soluble TCR binding to the same HLA-A2-flu complex. (A) TCR or TCR plus 120 μM soluble CD8 were injected into a control flow cell coated with 4100 RU of an irrelevant protein (CD5) and a probe flow cell coated with 4700 RU of HLA-A2-flu. After subtraction of the background, the calculated equilibrium response values at different concentrations of TCR alone (open circles) or in combination with 120 μM soluble CD8 (closed circles) is shown. Also shown is the value of CD8 alone (open triangles) and the calculated difference between TCR+CD8 and TCR alone (open squares). (B) The time-dependence of the responses on 4700 RU of immobilized HLA-A2-flu of 49 μM TCR alone (open circles) or in combination with 120 μM CD8 (closed circles) at 25° C. and a flow rate of 5 μ/min is shown (The values are corrected for background contributions measured on 4100 RU of immobilized CD5); the off-rate of TCR is not affected by the simultaneous CD8 binding.

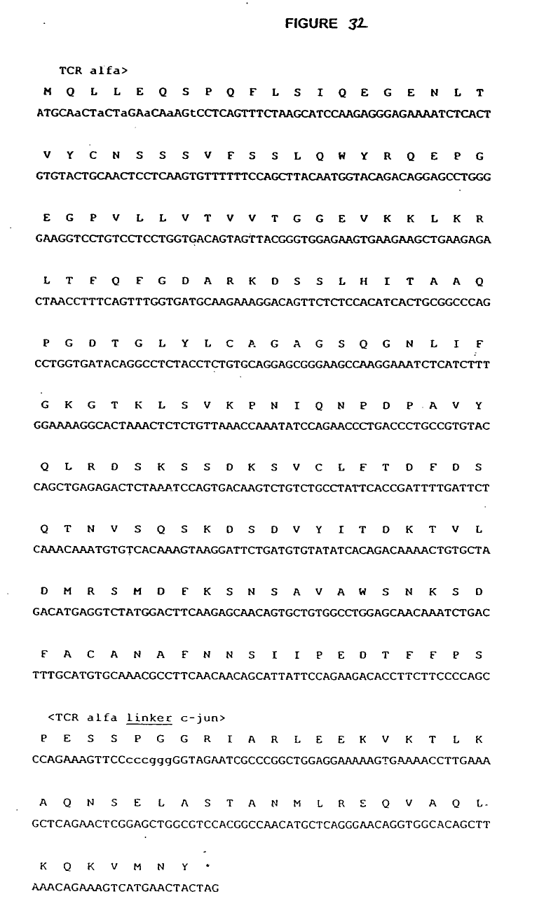

FIG. 32 shows the protein sequence (one-letter code, top) (SEQ ID NO. 66) and DNA sequence (bottom) (SEQ ID NO. 65) of the soluble, HLA-A2/flu matrix restricted TCR α chain from JM22, as fused to the “leucine zipper” domain of c-jun. Mutations introduced in the 5′ end of the DNA sequence to enhance expression of the gene in E. coli are indicated in small letters as is the linker sequence between the TCR and c-jun sequences.

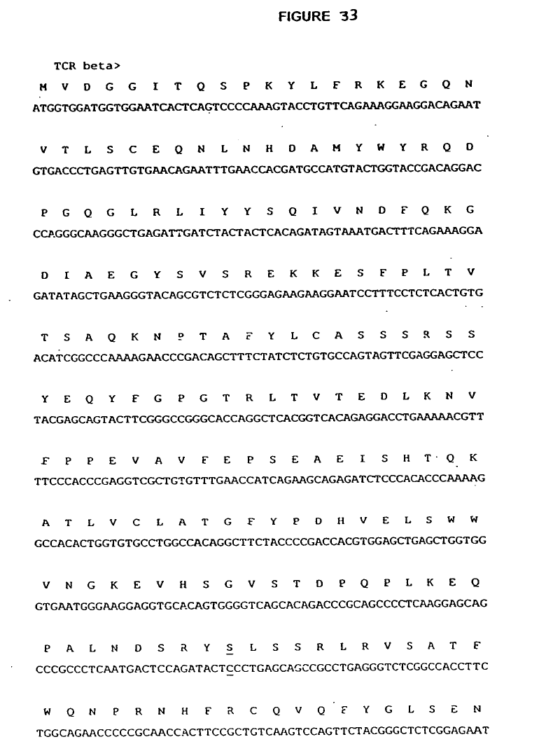

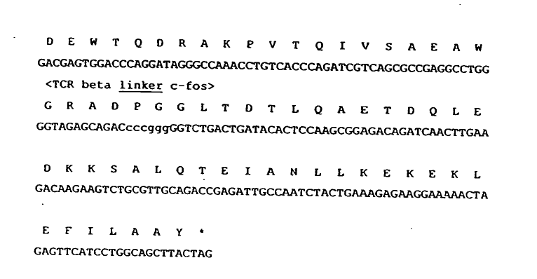

FIG. 33 show the protein sequence (one-letter code, top) (SEQ ID NO. 68) and DNA sequence (bottom) (SEQ ID NO. 67) of the soluble, HLA-A2/flu matrix restricted TCR β chain from JM22, as fused to the “leucine zipper” domain of c-fos. The linker sequence between the TCR and c-fos sequences is indicated in small letters. Mutation of the DNA sequence which substitutes a Serine residue for a Cysteine residue is indicated in bold and underlined. This mutation increases the folding efficiency of the TCR.

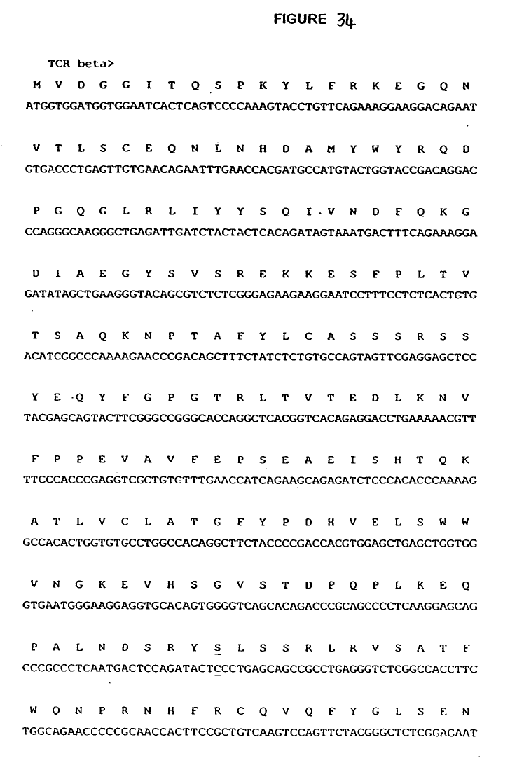

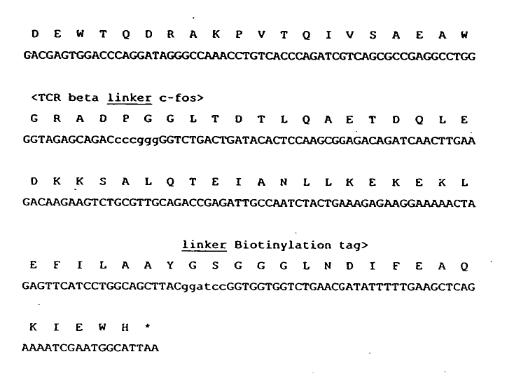

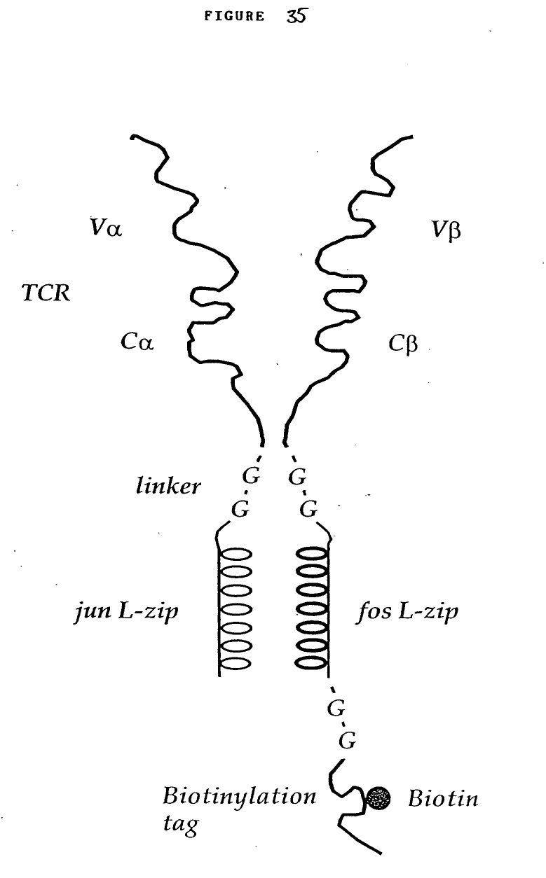

FIG. 34 shows the protein sequence (one-letter code, top) (SEQ ID NO. 70) and DNA-sequence (bottom) (SEQ ID NO. 69) of the soluble, HLA-A2/flu matrix restricted TCR β chain from JM22, as fused to the “leucine zipper” domain of c-fos and the biotinylation tag which acts as a substrate for BirA. The linker sequence between the TCR and c-fos sequences, and between c-fos and the biotinylation tag, are indicated in small letters. Mutation of the DNA sequence which substitutes a Serine residue for a Cysteine residue is indicated in bold and underlined. This mutation increases the folding efficiency of the TCR.

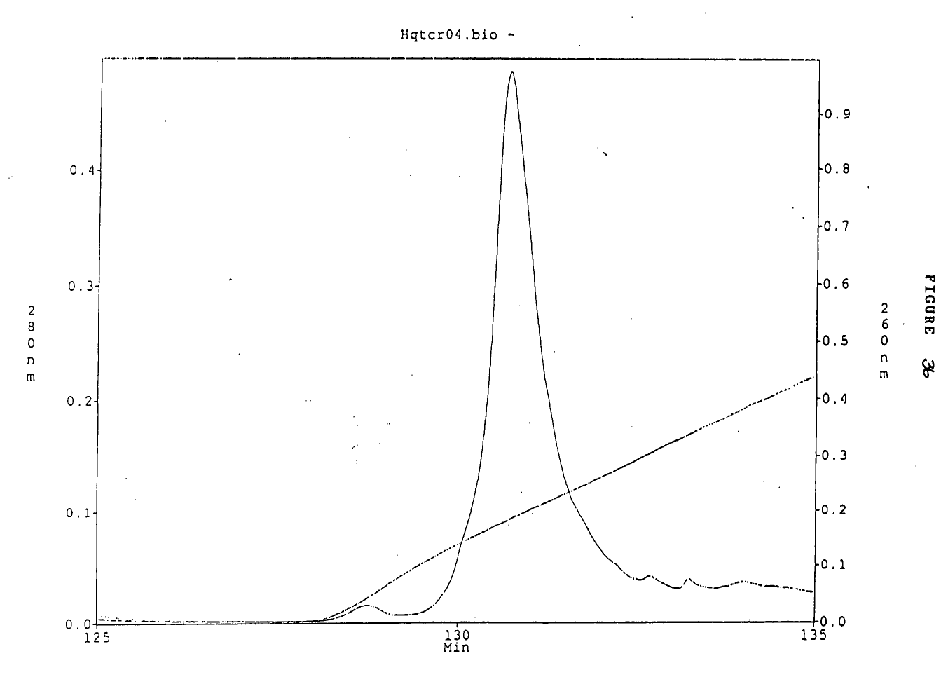

FIG. 35 is a schematic diagram of TCR-zipper-biotinylation tag fusion protein.

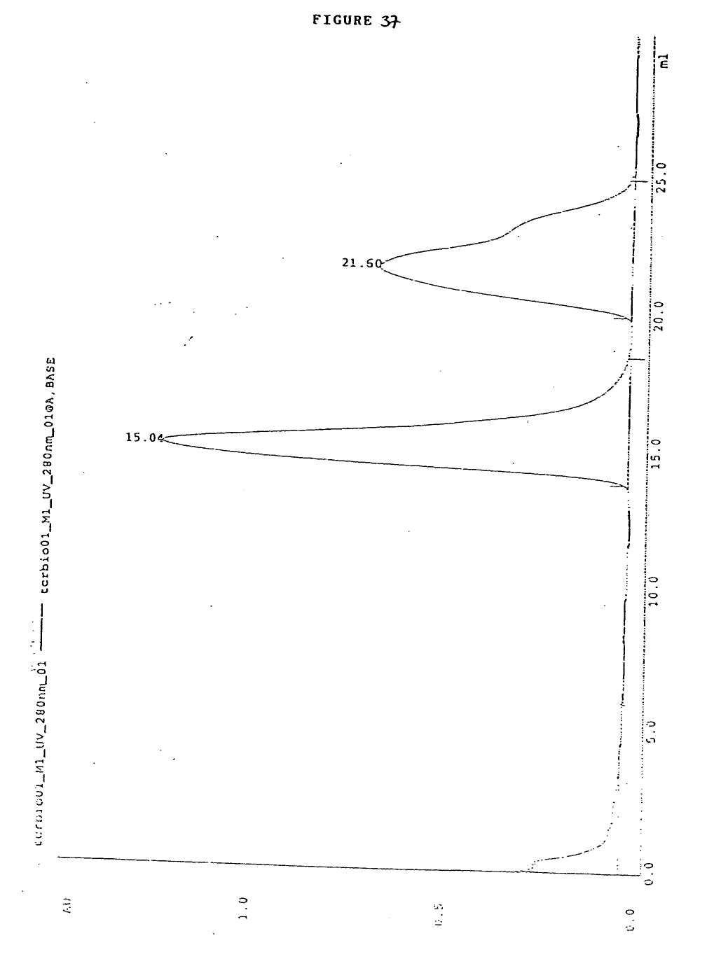

FIG. 36. Elution of refolded TCR from POROS™ 10HQ column with a gradient of sodium chloride. TCR elutes as a single peak at approximately 100 mM NaCl. Fractions containing protein with an OD(280 nm) of more than 0.1 were pooled and concentrated for biotinylation.

FIG. 37. Separation of biotinylated TCR from free biotin by gel filtration on a SUPERDEX™ 200HR 10/30 column (Pharmacia). TCR-biotin elutes at around 15 ml, corresponding to a molecular weight of 69 kDa. (Standard proteins and their elution volumes: Thyroglobulin (669 kDa) 10.14 ml, Apoferritin (443 kDa) 11.36 ml, beta-amylase (200 kDa) 12.72 ml, BSA dimer (134 kDa) 13.12 ml, BSA monomer (67 kDa) 14.93 ml, ovalbumin (43 kDa) 15.00 ml, chymotrypsinogen A (25 kDa) 18.09 ml, RNase A (13.7 kDa) 18.91 ml).

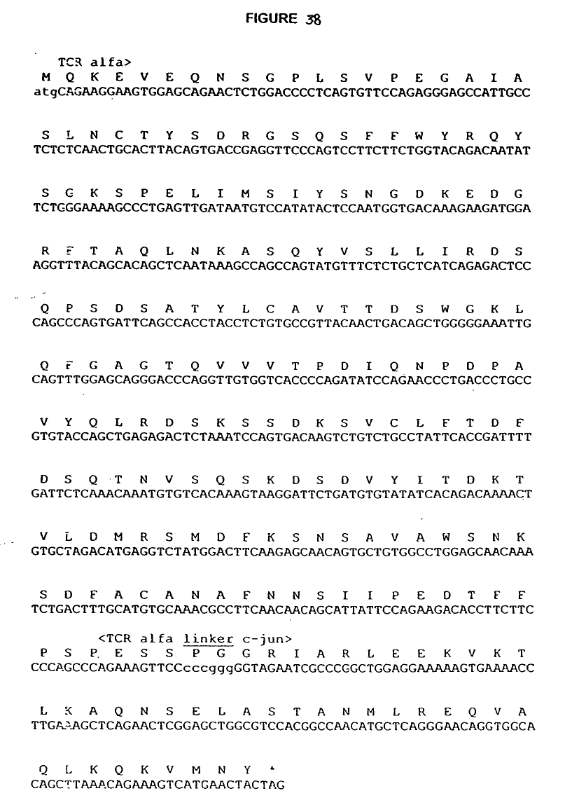

FIG. 38. Protein sequence (one-letter code, top) (SEQ ID NO. 72) and DNA sequence (bottom) (SEQ ID NO. 71) of the soluble, HTLV-1 Tax/HLA-A2 restricted TCR α chain from clone A6 (Garboczi et al., 1996a; Garboczi et al., 1996b), as fused to the “leucine zipper” domain of c-jun. Mutations introduced in the 5′ end of the DNA sequence to enhance expression of the gene in E. coli are indicated in small letters as is the linker sequence between the TCR and c-jun sequences.

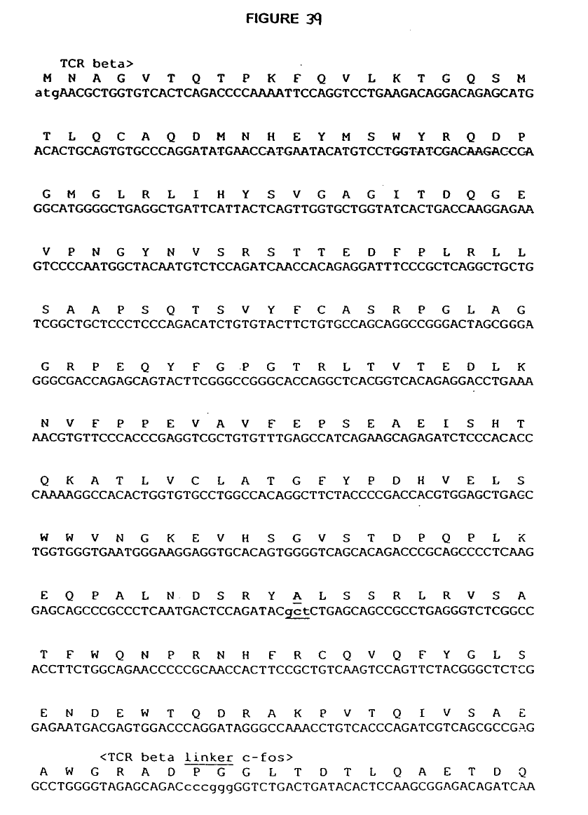

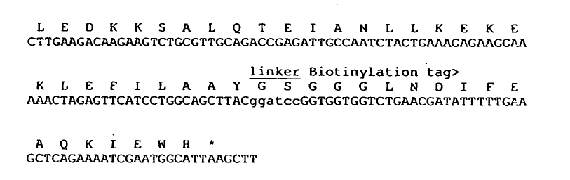

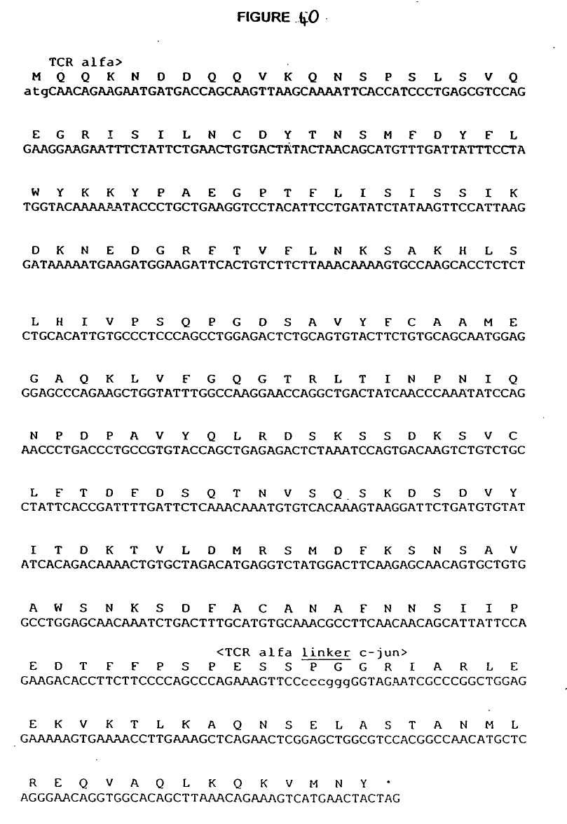

FIG. 39. Protein sequence (one-letter code, top) (SEQ ID NO. 74) and DNA sequence (bottom) (SEQ ID NO. 73) of the soluble, HTLV-1 Tax/HLA-A2 restricted TCR β chain from clone A6 (Garboczi et al., 1996a; Garboczi et al., 1996b), as fused to the “leucine zipper” domain of c-fos and the biotinylation tag which acts as a substrate for BirA. The linker sequence between the TCR and c-fos sequences is indicated in small letters. Mutation of the DNA sequence which substitutes an Alanine residue for a Cysteine residue is indicated in bold and underlined.

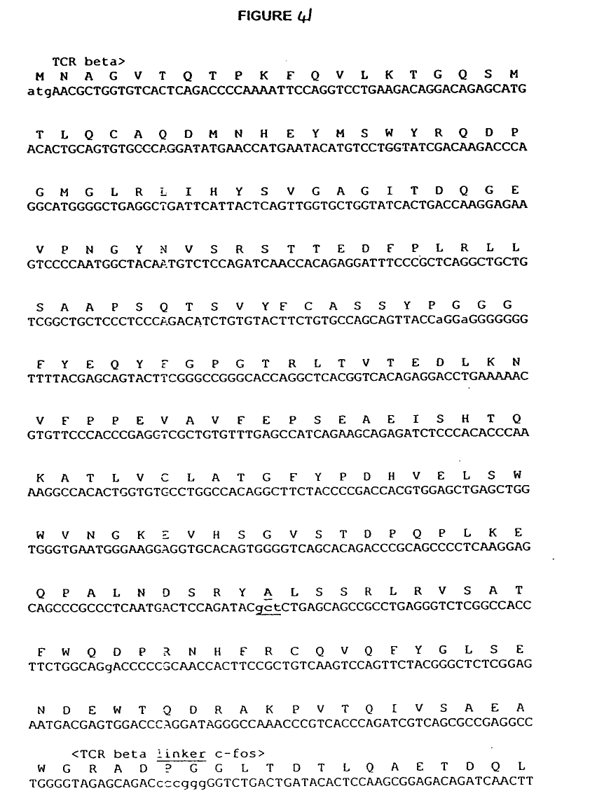

FIG. 40. Protein sequence (one-letter code, top) (SEQ ID NO. 76) and DNA sequence (bottom) (SEQ ID NO. 75) of the soluble, HTLV-1 Tax/HLA-A2 restricted TCR α chain from clone M10B7/D3 (Ding et al., 1998), as fused to the “leucine zipper” domain of c-jun. The linker sequence between the TCR and c-jun sequences is indicated in small letters.

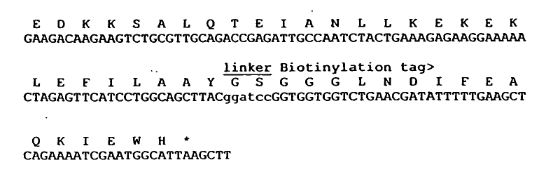

FIG. 41. Protein sequence (one-letter code, top) (SEQ ID NO. 78) and DNA sequence (bottom) (SEQ ID NO. 77) of the soluble, HTLV-1 Tax/HLA-A2 restricted TCR β chain from clone m1OB7/D3 (Ding et al., 1998), as fused to the “leucine zipper” domain of c-fos and the biotinylation tag which acts as a substrate for BirA. The linker sequence between the TCR and c-fos sequences is indicated in small letters. Mutation of the DNA sequence which substitutes an Alanine residue for a Cysteine residue is indicated in bold and underlined. Two silent mutations (P-G codons) introduced for cloning purposes and to remove a XmaI restriction site are also indicated in small letters.

### EXAMPLES

In the following examples, the general methods and materials set out below were used.

Materials

Restriction enzymes (NdeI, BamHI, HindIII, Bsu361, XmaI) were from New England Biolabs.

Tris pH 8.1 was made up as a 2M stock solution from equal parts of Tris base and Tris-HCl both from USB.

EDTA (Sigma) was made up as a 0.5M stock solution and the pH was adjusted to 8.0 using 5M NaOH (Sigma).

Glutathione in oxidized and reduced forms was from Sigma.

Cystamine and cysteamine were from Sigma.

Sodium Chloride was from USB and was made up to a 4M stock solution.

Miniprep kits for plasmid purification were from Quiagen.

PCR purification kits were from Quiagen.

DTT was from Sigma.

Guanidine was from Fluka.

Urea was from Sigma.

RPM1 medium was from Sigma.

PBS was made up from tablets from Oxoid.

Glycerol was from BDH.

General Methods

Bacterial media (TYP media) were prepared as follows:

160 g Yeast Extract (Difco), 160 g Tryptone (Difco), 50 g NaCl (USB) and 25 g K₂HPO₄ (BDH) were dissolved in 2 L demineralized water. 200 ml aliquots of this solution were measured into 10×2 L conical flasks and made up to 1 L by adding 800 ml demineralized water. Flasks were covered with four layers of aluminum foil, labelled and autoclaved. After cooling, the flasks were stored at room temperature out of direct sunlight prior to use.

Protein concentrations were measured using a Pierce Coomassie-binding assay and BSA as a standard protein. Briefly, 0-25 μg BSA standards in a volume of 1 ml water were prepared from a stock 2 mg/ml BSA (Pierce) in 4 ml plastic cuvettes. Approximately 10 μg of unknown protein was made up to 1 ml with water in the same way. 1 ml Pierce Coomassie reagent was added to each cuvette and the contents were thoroughly mixed. The optical density was measured within 15 minutes at 595 nm using a Beckman DU-530 UV spectrophotometer. A linear regression was performed on the results from the BSA standards (linearity was good up to 25 μg BSA) and the unknown protein concentration was estimated by interpolation with these results.

Gel filtration chromatography was performed on a Pharmacia FPLC system equipped with a computer controller. Protein elution was monitored using a UV-M 11 system measuring absorbance at 280 nm wavelength. For small-scale separations, a SUPERDEX™ 200 HR 10/30 column was employed and sample was loaded using a 1 ml loop. Prior to running the column was equilibrated with 30 ml of PBS and the sample was run at 0.5 ml/min with 1 ml fractions being collected. For large-scale separations, a SUPERDEX™ 75 or 200 PG 26/60 column was used with a 10 ml superloop. In this case 5 or 10 ml samples were collected and the column was run at 4 ml/min. All separations were performed at room temperature.

Ion exchange chromatography was performed on a BIOCAD™ Sprint system (Perkin-Elmer). For cation exchange, a 20 HS or a 50 HS column was employed. For anion exchange, a 10 HQ, 20 HQ or a 50 HQ column was employed. Columns were run using the recommended buffers attached to a 6-way mixer. Small samples (5-25 ml) were injected using a 5 ml injection loop. Larger samples (>100 ml) were injected using one of the buffer lines. 1 ml fractions were collected during the elution phase of the column run. Protein elution was measured by in-line absorbance at 280 nm.

SDS polyacrylamide gel eletrophoresis (SDS-PAGE) was performed using a Bio-Rad MINI-PROTEAN™ II gel set. Gels were poured prior to use using the following procedure. The gel plate assembly was prepared and checked to ensure against leakage. Then the following mixture was prepared: 12% acrylamide/bisacrylamide (from a 30% acrylamide/0.8% bisacrylamide stock solution (National Diagnostics)), 0.375 M Tris pH 8.8 (from a 1.5 M stock of the same pH), 0.1% SDS (from a 10% SDS stock solution), 0.05% Ammonium persulphate (from a 10% stock of the same, stored at 4 C) and 0.1% TEMED (Sigma). The mixture was immediately poured into the gel plate assembly and water-saturated butanol was layered on top to ensure a flat upper surface. After the gel had set (10-15 minutes minimum), the stacking gel was mixed as follows. 4% acrylamide (from stock as before), 0.125 M Tris pH 6.8 (from 0.5 M stock of the same pH), 0.1% SDS, 0.05% Ammonium persulphate, and 0.2% TEMED. The butanol was removed from the surface of the resolving gel by absorption onto a tissue and the stacking gel mixture was poured on top of the resolving gel. A gel comb was immediately inserted taking care to avoid introducing air bubbles into the gel and the stacking gel was allowed to set for a minimum of 5 minutes.

The gel was then assembled into the gel apparatus and running buffer (3 g/L Tris-base, 14.4 g/L glycine, 1 g/L SDS (diluted from a 10× concentrated stock solution) was poured into the apparatus at the anode and the cathode. After removing the gel comb, the wells were washed out with running buffer to prevent residual acrylamide mixture from setting in the bottom of the wells. Samples were prepared by mixing protein 1:1 with the following mixture: 4% SDS, 0.125 M Tris pH 6.8, 20% glycerol, 10% β-mercaptoethanol, 1% bromophenol blue (Sigma). Samples were then heated to 95° C. for 2 minutes and cooled prior to loading up to 25 μl into the wells in the stacking gel. Approximately 1-10 μg of protein was usually loaded to ensure good staining and running of the gel. After loading, the gels were run at a constant voltage of 200 V for approximately 40 minutes or until the bromophenol blue dye was approximately 5 mm from the end of the gel.

After completing of the electrophoresis, the gels were removed from the apparatus and carefully dropped into a 0.1% solution of Coomassie R-250 (Sigma) in 10% acetic acid, 40% methanol, 50% water. Gels were then gently agitated for at least 30 minutes prior to destaining in several changes of 10% acetic acid, 40% methanol, 50% water until the gel background was clear. Gels were then stored in water and recorded using a UVP gel documentation system consisting of a light box, a digital camera and a thermal printer.

### Example 1

### Recombinant Soluble TCR

A recombinant soluble form of the heterodimeric TCR molecule was engineered as outlined in FIG. 1. Each chain consists of membrane-distal and -proximal immunoglobulin domains which are fused via a short flexible linker to a coiled coil motif which helps stabilize the heterodimer.

The TCR constant domains have been truncated immediately before cysteine residues which in vivo form an interchain disulfide bond. Consequently, the two chains pair by non-covalent quaternary contacts, and this is confirmed in FIG. 2b. As the Fos-Jun zipper peptide heterodimers are also capable of forming an interchain disulfide immediately N-terminal to the linker used (O'Shea et al. 1989), the alignment of the two chains relative to each other was predicted to be optimal. Fusion proteins need to be joined in a manner which is compatible with each of the separate components, in order to avoid disturbing either structure.

cDNA encoding alpha and beta chains of a TCR specific for the influenza-matrix protein 58-66 epitope in HLA-A2 was obtained from a Vβ17+human CTL clone (JM22) by anchored PCR as described previously (Moss et al. 1991).

α and β TCR-zipper constructs pJM22α-Jun and pJM22α-Fos were separately constructed by amplifying the variable and constant domain of each chain using standard PCR technology and splicing products onto leucine zipper domains from the eukaryotic transcription factors Jun and Fos respectively (See FIG. 1). These 40 amino acid long sequences have been shown to specifically heterodimerize when refolded from synthetic peptides, without the need for a covalent interchain linkage (O'Shea et al. 1989).

Primers were designed to incorporate a high AT content immediately 3′ to the initiation codon (to destabilize mRNA secondary structure) and using E. coli codon preferences, in order to maximize expression (Gao et al. 1998). The spare cysteine in the TCR beta constant domain was mutated to serine to ensure prevention of incorrect disulfide bonding during refolding.

DNA constructs were ligated separately into the E. coli expression vector pGMT7. Plasmid digests and DNA sequencing confirmed that the constructs were correct.

In detail the procedures used were as follows.

Expression of TCR zipper chains and purification of denatured inclusion bodies: GFG020 and GFG021, the pGMT7 expression plasmids containing JM22α-Jun and JM22β-Fos respectively were transformed separately into E. coli strain BL21 pLysS, and single ampicillin-resistant colonies were grown at 37° C. in TYP (ampicillin 100 μg/ml) medium to OD₆₀₀ of 0.4 before inducing protein expression with 0.5 mM IPTG. Cells were harvested three hours post-induction by centrifugation for 30 minutes at 4000 rpm in a Beckman J-6B. Cell pellets were resuspended in a buffer containing 50 mM Tris-HCl, 25% (w/v) sucrose, 1 mM NaEDTA, 0.1% (w/v) NaAzide, 10 mM DTT, pH 8.0. After an overnight freeze-thaw step, resuspended cells were sonicated in 1 minute bursts for a total of around 10 minutes in a Milsonix XL2020 sonicator using a standard 12 mm diameter probe. Inclusion body pellets were recovered by centrifugation for 30 minutes at 13000 rpm in a Beckman J2-21 centrifuge. Three detergent washes were then carried out to remove cell debris and membrane components. Each time the inclusion body pellet was homogenized in a Triton buffer (50 mM Tris-HCl, 0.5% Triton-X100, 200 mM NaCl, 10 mM NaEDTA, 0.1% (w/v) NaAzide, 2 mM DTT, pH 8.0) before being pelleted by centrifugation for 15 minutes at 13000 rpm in a Beckman J2-21. Detergent and salt was then removed by a similar wash in the following buffer: 50 mM Tris-HCl, 1 mM NaEDTA, 0.1% (w/v) NaAzide, 2 mM DTT, pH 8.0. Finally, the JM22α-Jun and JM22α-Fos inclusion body pellets were dissolved separately in a urea solution (50 mM MES, 8 M urea, 10 mM NaEDTA, 2 mM DTT, pH 6.5) for 3 to 4 hours at 4° C.

Insoluble material was pelleted by centrifugation for 30 minutes at 13000 rpm in a Beckman J2-21, and the supernatant was divided into 1 ml aliquots and frozen at −70° C. Inclusion bodies solubilized in urea were quantitated with a Bradford dye-binding assay (Biorad). For each chain a yield of around 100 mg of purified inclusion body was obtained from one litre of culture. Each inclusion body (JM22α-Jun, JM22β-Fos) was solubilized in urea solution at a concentration of around 20 mg/ml, and was estimated from gel analysis to be around 90% pure in this form (data not shown).

Co-Refolding of TCR-Zipper Fusion Proteins:

Initial refolding experiments using a standard refolding buffer (100 mM Tris pH 8.5, 1 M L-Arginine, 2 mM EDTA, 5 mM reduced Glutathione, 0.5 mM oxidized Glutathione, 0.2 mM PMSF) resulted in severe protein precipitation which was dependent upon the presence of the zipper domains. The fact that this phenomenon occurred at concentrations below the dissociation constant of zipper dimerization (i.e. when most zipper helices are expected to be monomeric) suggested additional forces were stabilizing misfolded species. The most likely explanation is that the entirely alpha-helical zipper domains fold first and that their transient heterodimerization induces inter-chain aggregation of partially folded intermediates of the more complex immunoglobulin domains. The refolding buffer was therefore altered to include 5 M urea in order to prevent hydrophobic interactions between partially folded immunoglobulin domains and allow individual chains to fold completely before heterodimerization. This step is sufficient to prevent precipitation occurring, and allows correctly folded TCR-zipper heterodimers to assemble with acceptable yields using the following protocol.

Urea-solubilized stocks of TCR-zipper chains JM22α-Jun and JM22β-Fos were renatured by dilution co-refolding. Approximately 30 mg (i.e. 1 μMole) of each solubilised inclusion body chain was thawed from frozen stocks and a further pulse of DTT (4 μmoles/ml) was added to ensure complete reduction of cysteine residues. Samples were then mixed and the mixture diluted into 15 ml of a guanidine solution (6 M Guanidine-hydrochloride, 10 mM Sodium Acetate, 10 mM EDTA), to ensure complete chain denaturation. The guanidine solution containing fully reduced and denatured TCR-zipper chains was then injected into 1 litre of the following refolding buffer: 100 mM Tris pH 8.5, 400 mM L-Arginine, 2 mM EDTA, 5 mM reduced Glutathione, 0.5 mM oxidized Glutathione, 5M urea, 0.2 mM PMSF. The solution was left for 24 hrs. The refold was then dialyzed twice, firstly against 10 litres of 100 mM urea, secondly against 10 litres of 100 mM urea, 10 mM Tris pH 8.0. Both refolding and dialysis steps were carried out at 6-8° C.

Purification of Refolded TCR-Zipper:

TCR-zipper JM22zip was separated from degradation products and impurities by loading the dialyzed refold onto a POROS™ 10HQ analytical anion exchange column in seven 200 ml aliquots and eluting bound protein with a gradient of 0-400 mM NaCl over 50 column volumes using a BIOCAD™ workstation (Perseptive Biosystems). Non-covalently associated heterodimer eluted in a single peak at approximately 100 mM NaCl. Peak fractions (typically containing heterodimer at a concentration of 100-300 μg/ml) were stored at 4° C. before being pooled and concentrated. The yield of heterodimer is approximately 15%.

Characterisation of the Refolded TCR-Zipper JM22zip:

The JM22zip heterodimer purified by anion exchange elutes as an approximately 70 kDa protein from a SUPERDEX™ 200 gel filtration sizing column (Pharmacia). It is especially important to include gel filtration steps prior to surface plasmon resonance binding analysis since accurate affinity and kinetic measurements rely on monomeric interactions taking place. In this way, higher order aggregates can be excluded from the soluble protein fraction used for analysis. In particular, aggregates cause artifactually slow association and dissociation rate constants to be detected.

The oxidation state of each chain has been examined by a reducing/non-reducing gel analysis in FIG. 2. In the presence of SDS, the non-covalently associated heterodimer is dissociated into alpha and beta chains. If DTT is used in loading buffer, the two chains run either side of the 31 kDa marker. In the absence of such denaturants both chains still behave as a single species, but the mobility of each increases, which suggests each chain has formed a single, disulfide-bonded species (Garboczi et al. 1996).

The antibody reactivity of refolded receptor has been tested using surface plasmon resonance on a Biacore 2000 machine (Biacore). The TCR-zipper JM22z was immobilized to a dextran matrix (CM chip) binding surface at pH 5.5 using standard amine coupling methods. A variable region antibody specific for the beta chain (Vβ17) specifically binds to the immobilized receptor, implying correct conformation.

Stability:

The soluble TCRs expressed as alpha-jun and beta-fos leucine zipper fusions are stable over periods of months and are therefore suitable for the detection of specific antigens presented by class I MHC.

### Example 2

### Kinetics and Affinity Study of Human TCR-Viral Peptide-MHC

Specific Binding of Refolded TCR-Zipper to Peptide-MHC Complexes:

A surface plasmon resonance biosensor (Biacore) was used to analyze the binding of a TCR-zipper (JM22zip, specific for HLA-A2 influenza matrix protein M58-66 complex) to its peptide-MHC ligand (see FIG. 3). We facilitated this by producing single pMHC complexes (described below) which can be immobilized to a streptavidin-coated binding surface in a semi-oriented fashion, allowing efficient testing of the binding of a soluble T-cell receptor to up to four different pMHC (immobilized on separate flow cells) simultaneously. Manual injection of HLA complex allows the precise level of immobilized class I molecules to be manipulated easily.

Such immobilized complexes are capable of binding both T-cell receptors (see FIG. 3) and the coreceptor CD8αα, both of which may be injected in the soluble phase. Specific binding of TCR-zipper is obtained even at low concentrations (at least 40 μg/ml), implying the TCR zipper is relatively stable. The pMHC binding properties of JM22z are observed to be qualitatively and quantitatively similar if TCR is used either in the soluble or immobilized phase. This is an important control for partial activity of soluble species and also suggests that biotinylated pMHC complexes are biologically as active as non-biotinylated complexes.

Preparation of Chemically Biotinylated HLA Complexes:

Methods for the production of soluble, recombinant single peptide class I HLA complexes have already been described (Garboczi et al. 1992). These have been modified in order to produce HLA complexes which have β-2-microglobulin domains chemically biotinylated and may therefore be immobilized to a streptavidin coated binding chip and used for surface plasmon binding studies.

β-2-microglobulin was expressed and 40 mg refolded in a standard refolding buffer (100 mM Tris pH 8.0, 400 mM L-Arginine, 2 mM EDTA, 5 mM reduced Glutathione, 0.5 mM oxidised Glutathione, 0.1 mM PMSF) essentially as described (Garboczi et al. 1992). After an optional gel filtration step, protein was exchanged to 0.1 M Sodium Borate pH 8.8, and finally concentrated to 5-10 mg/ml. β-2-microglobulin was also quantitated using the Bradford assay (Biorad). A 5 molar excess of biotin hydroxysuccinimide (Sigma) was added from a stock made up at 10 mg/ml in DMSO. The reaction was left for 1 hour at room temperature, and stopped with 20 μl of 1 M Ammonium Chloride/250 μg of biotin ester used. Refolded HLA complex was separated from free biotin and free biotinylated beta-2-microglobulin using a SUPERDEX™ 200 gel filtration sizing column (Pharmacia). Streptavidin was immobilized by standard amine coupling methods.

### CONCLUSIONS

Thus, the protein refolding methods described in Example 1 produce a stable, correctly folded, functional recombinant receptor fusion protein which is suitable for biophysical analysis using an optical biosensor. This has provided a reagent used to carry out a detailed affinity and kinetic analysis of a human TCR-pMHC interaction. The effects of T-cell co-receptor-MHC and TCR-pMHC interactions on each other have also been studied. The recombinant techniques used are applicable in principle to both murine and human TCRs, both class I and class II—restricted, and will enable similar analyzes of a range of TCRs. This would allow various questions to be addressed, such as the span of TCR affinities within an antiviral response, the properties of dominantly selected receptors and the kinetic requirements for receptor triggering. The methods also provide a way of verifying the ligand specificity of a TCR prior to crystallization trials, and may also have implications for the recombinant production of other cell surface receptors.

### Example 3

### Biotinylation and Tetramerization of Soluble T-Cell Receptors

2.5 ml purified soluble TCR prepared as described in Example 1 (˜0.2 mg/ml) was buffer exchanged into biotinylation reaction buffer (10 mM Tris pH 8.0, 5 mM NaCl, 7.5 mM MgCl₂) using a PD-10 column (Pharmacia). The eluate (3.5 ml) was concentrated to 1 ml using a CENTRICON™ concentrator (Amicon) with a 10 kDa molecular weight cut-off. This was made up to 5 mM with ATP added from stock (0.1 g/ml adjusted to pH 7.0). A cocktail of protease inhibitors was added: leupeptin, pepstatin and PMSF (0.1 mM), followed by 1 mM biotin (added from 0.2 M stock) and 5 μg/ml enzyme (from 0.5 mg/ml stock). The mixture was then incubated overnight at room temperature. Excess biotin was removed from the solution by dialysis against 10 mM Tris pH 8.0, 5 mM NaCl (200 volumes, with 2 changes at 4° C.). The protein was then tested for the presence of bound biotin by blotting onto nitrocellulose followed by blocking with 5% skimmed milk powder, and detection using streptavidin-HRP conjugate (Biorad). Tetramerization of the biotinylated soluble TCR was with either extravidin-RPE or extravidin-FITC conjugate (Sigma). The concentration of biotin-soluble TCR was measured using a Coomassie binding protein assay (Pierce), and a ratio of extravidin conjugate to soluble TCR of 0.224 mg 1 mg TCR was calculated to achieve saturation of the extravidin by biotinylated TCR at a ratio of 1:4. The extravidin conjugate was added in aliquots of 1/10th of the total added, on ice, for at least 15 minutes per aliquot (to ensure saturation of the extravidin). Soluble TCR tetramers were stored at 4° C. in the dark. The tetramers are extremely stable over a period of months.

### Example 4

### Molecular Cloning of T Cell Receptor Genes from T Cell Lines or T Cell Clones of Known Specificity.

The methods and procedures for molecular cloning of TCR genes from cells is identical for all α chains and for all β chains, respectively, and are therefore only described in this example.

A suitable number of T cells, typically 1-5 million, were lysed in Lysis Buffer from the ‘mRNA Capture Kit’ (Boehringer Mannheim). mRNA was isolated with kit reagents by hybridizing biotinylated oligo-dT to the poly-A tails of the mRNA. The hybridized complexes were then captured by binding of biotin to a PCR tube coated with streptavidin. Following immobilization of the mRNA in the PCR tube, cDNA was generated using AMV reverse transcriptase (Stratagene) as described (Boehringer Mannheim manual for ‘mRNA Capture Kit’).

With the cDNA still immobilized, a poly-G tails were generated at the 3′ ends using the Terminal Transferase enzyme (Boehringer Mannheim). PCR reaction mix was then added, including the high fidelity thermostable polymerase pfu (cloned, Stratagene), which was used in order to minimize the risk of errors in the PCR products. PCR reactions were performed using a poly-C ‘anchor primer’ (FIG. 4A) and α or β chain specific primers (FIGS. 4B and C, respectively) annealing in the respective TCR constant regions. PCR reactions of 30 cycles of denaturation at 95° C. for 1 minute, annealing at 50° C. for 1 minute, and extensions at 72° C. for 5 minutes were performed to amplify TCR gene fragments.

PCR products were ligated into a BLUESCRIPT™ sequencing vector (PBLUESCRIPT™ II KS-, Stratagene) using the XhoI and XmaI restriction enzyme sites contained in the PCR primers (all enzymes from New England Biolabs). Following transfection of the ligation mixes in the E. coli strain XL-1 Blue, several clones for each chain were selected for DNA sequencing which was performed on an ABI 377 Prism automatic sequencer using BIGDYE™ terminators (Applied Biosystems Inc.).

### Example 5

### Molecular Cloning of DNA Fragments Encoding the 40 Amino Acid Coiled-Coil (‘Leucine Zipper’) Regions of c-jun and c-fos.

DNA fragments encoding the 40 amino acid coiled-coil (‘leucine zipper’) regions of c-jun and c-fos were generated by PCR reactions using human cDNA as template and the primers shown in FIG. 5. PCR reactions were carried out in reaction buffer including cloned pfu polymerase (Stratagene) for 30 cycles consisting of denaturation at 95° C. for 1 minute, primer annealing at 58° C. for 1 minute, and extension at 72° C. for 2 minutes.

The c-jun and c-fos fragments were ligated into PBLUESCRIPT™ II KS-(Stratagene) using the unique XhoI and XmaI restriction sites to obtain constructs pBJ107 and pBJ108, respectively (FIG. 6). The DNA sequences of the c-jun and c-fos fragments were verified by DNA sequencing performed on an ABI 377 Prism automatic sequencer using BIGDYE™ terminators (Applied Biosystems Inc.).

The sequenced c-jun and c-fos fragments were then subcloned, using the unique XmaI and BamHI restriction sites, into the polylinker region of the T7 polymeraze expression vector, pGMT7 (Studier, Rosenberg et al. 1990).

### Example 6

### Design of TCR-Leucine Zipper Fusion Proteins for the Production of Stable, Soluble TCRs

Attempts to co-refold extracellular fragments of TCR α and β chains, truncated so that they contained the cysteine residue which in vivo forms a disulfide bond, produced limited success (data not shown, see Example 9 for expression methods and general methods and materials for refolding conditions). However, when the TCR α and β chains were truncated immediately before, that is on the N-terminal side of, the cysteine residue forming the interchain disulfide bond, analytical chromatography on a SUPERDEX™ G-75 column (Pharmacia) indicated that a small fraction of protein, approximately 1-2% of the amount used in the refolding reaction, had refolded into a complex of the expected molecular size for the truncated α/β heterodimer (see also (Garboczi, Utz et al. 1996) for reference to method).

Because incorrect disulfide bond formation can cause irreversible misfolding of protein during in vitro refolding, the probabilities for this to happen were sought to be minimized by mutating a cysteine residue in the TCR β constant region which is unpaired in the cellular TCR. The cysteine residue is substituted for a serine or an alanine reside. The synthetic DNA primers used for these mutation steps are shown in FIG. 7. Co-refolding of TCR α and mutated β chains, both truncated immediately before the cysteine residue which forms the interchain disulfide bond, showed a dramatic improvement in yields of heterodimer, the protein fraction of correct molecular weight typically constituting 15-30% of total protein. However, when these soluble TCRs were stored overnight, analysis of the protein showed that the fraction with a molecular weight corresponding to the heterodimeric TCR had split into two peaks of molecular weight corresponding to the monomeric TCR α and β chains. Similar observations were made upon dilution of the soluble TCRs, indicating that α/β chain stability was low and insufficient for analyzes which would require a timespan longer than a limited number of hours or dilution of the protein. In conclusion, these methods for producing soluble TCR only generated receptor with extremely limited stability.

To improve TCR α/β chain stability, and to potentially aid heterodimer formation during refolding, the TCR chains were fused to the ‘leucine zipper’ domains of c-jun and c-fos which are known preferentially to form heterodimers (O'Shea, Rutkowski et al. 1989; Schuermann, Hunter et al. 1991; O'Shea, Rutkowski et al. 1992; Glover and Harrison 1995). Two designs for the fusion TCRs were tested.

In one, the leucine zippers were fused just after, that is C-terminal to, the cysteine residues forming the interchain disulfide bond in the TCR α and β chains. As the c-jun and c-fos leucine zipper peptides are also capable of forming an interchain disulfide immediately N-terminal to the linker used (O'Shea, Rutkowski et al. 1989), the alignment of the two chains relative to each other, and to the interchain disulfide bond, was predicted to be optimal.

In the other design, the leucine zippers were fused just before, that is N-terminal to, the cysteine residues forming the interchain disulfide bond in the TCR α and β chains (FIG. 8). Thus, in the second design the cysteine residues are omitted from the recombinant receptor.

In refolding experiments with TCR-zipper (TCR-z) chains of these designs, it was found that the yield of heterodimeric, soluble receptor was better when the cysteine residues forming the interchain disulfide bond were omitted from the TCR α and β chains, as in the design shown in FIG. 8.

### Example 7

### Construction of DNA Expression Vectors for TCR-Leucine Zipper Proteins

This example describes the construction of expression vectors for the α and β chains of five TCRs. The strategy and design described should be adaptable to any human or animal TCR genes. Although the five TCRs described here are all restricted by MHC class I epitopes, the methods could be identically employed for the cloning and construction of expression vectors for MHC class II restricted TCRs. All vectors express protein aimed for refolding soluble TCRs according to the design shown in FIG. 9, with the exception that two TCRs were expressed with a biotinylatable tag sequence at the C-terminus (see below and FIGS. 17, 18, and 19). The cloning strategies are identical for all TCR α and β chains, respectively.

The extent of the leader, or signal, peptide sequences of TCR α and β chains were predicted from analyzes of the sequence data obtained from plasmids containing TCR anchor PCR products (see Example 4). On this basis, 5′ primers for generating PCR fragments for the expression of TCR chains without leader sequences were designed (FIG. 9). All 5′ primers encode a methionine residue just prior to the mature TCR protein sequences in order to allow translation in E. coli. Silent mutations, substituting C or G bases for A or T (FIG. 9), were introduced in a number of the 5′ proximal codons of the genes in order to decrease the tendency for secondary mRNA structure formations which could adversely inhibit expression levels in E. coli (PCT/GB 98/03235; (Gao, Tormo et al. 1997; Gao, Gerth et al. 1998).

The genes encoding the Vα0.2 and the Vβ17 chains of the human JM22 Influenza Matrix peptide-HLA-A0201 (peptide sequence GILGFVFTL) (SEQ ID NO. 79) restricted TCR, the human Vα23 and the Vβ5.1 chains of the 003 HIV-1 Gag peptide-HLA-A0201 (peptide sequence SLYNTVATL) (SEQ ID NO. 80) restricted TCR, and the murine Vα4 and Vβ11 chains of the F5 NP peptide-H2-^{Db} (peptide sequence ASNENMDAM) (SEQ ID NO. 81) were amplified by PCR using plasmids containing TCR anchor PCR products generated as described in Example 6. The genes for the human A6 (Vα2.3/Vβ12.3) and B7 (Vα17.2/Vβ12.3) TCRs which are specific the HTLV-1 Tax peptide presented by HLA-A0201 (peptide sequence LLFGYPVYV) (SEQ ID NO. 82), were obtained in plasmid form (Garboczi, Utz et al. 1996; Ding, Smith et al. 1998) which were used for the generation of PCR products for the construction of expression vectors for these TCR chains. The genes for these TCRs were cloned into expression vectors that contained the sequence for a c-fos leucine zipper-biotinylatable tag fusion fragment (see Example 8).

PCR reactions were performed with cloned pfu polymeraze at standard buffer conditions (Stratagene) and with 25 cycles of denaturation at 95° C. for 1 minute, primer annealing at 60° C. for 1 minute, and extensions at 72° C. for 6 minutes. The PCR products were restriction digested with the enzymes NdeI and XmaI and ligated into the pGMT7 vectors containing the c-jun (TCR α chains) and c-fos (TCR β chains) inserts (see Example 5).

FIGS. 10-19 show the sequences of the TCR-z inserts and the predicted protein sequences expressed by the pGMT7 vectors. FIG. 20 shows the sequence of the A6 TCR β chain containing a mutation in the constant region but which did not detectably affect the folding and function of the soluble TCR (see Examples 9 and 10).

### Example 8

### Construction of DNA Vectors for the Expression of TCR β Chains Fused to a c-fos Leucine Zipper-Biotinylatable Fragment

In order to enable soluble TCRs to be immobilized or to allow detection or attachments to the receptor, it would be useful if the protein could be produced with a further functional fusion component. This could allow the soluble TCR to be derivatised, such as to be produced as multimers, or allow detection with high sensitivity, or attach other functions to the receptor/receptor complexes.

This example demonstrates the construction of expression vectors for TCR β chains onto which is engineered a fusion polypeptide which can be specifically biotinylated in E. coli in vivo or with the enzyme BirA in vitro (Barker and Campbell 1981; Barker and Campbell 1981; Howard, Shaw et al. 1985; Schatz 1993; O'Callaghan, Byford et al. 1999). As shown in Examples 10 and 11, these soluble TCR fusions can be expressed and refolded together with α chain in an identical manner and with similar yields to the TCR β chain which is not fused to the ‘biotinylation tag’ (BT-tag). These results demonstrate that the soluble TCR described herein is likely to be suitable for expression with a multitude of different polypeptides as fusion partners.

T Cell Receptor β-chains were sub-cloned into a pGMT7 expression vector with a biotin-tag sequence C-terminal to the fos leucine zipper sequence as follows:

start—TCR β-chain—fos zipper—biotin-tag—stop

The exact sequence of the ends of the constructs was as follows (see also FIG. 21):
Linker [symbol] fos zipper [symbol] BamHI [symbol] linker [symbol] [symbol] biotin tag

Two approaches were used to produce soluble TCRs with the biotin tag. In the case of the human JM22 Influenza Matrix peptide-HLA-A0201 restricted TCR, the cloned β-chain-c-fos leucine zipper fusion was modified at the 3′-end using the synthetic DNA primer shown in FIG. 22 to introduce a BamHI site instead of a HindIII site using a standard PCR reaction with pfu polymerase (Stratagene).

The original 5′ primer (see FIG. 9) containing an NdeI site was used as the forward primer. The PCR product produced was cloned into a modified pGMT7 vector containing the biotin-tag sequence (FIG. 21) to form the construct outlined above. This plasmid is known as JMB002.

The cloned TCR specific for the HLA-A0201 restricted HTLV-1 epitope LLFGYPVYV (SEQ ID NO. 82), known as the A6 tax TCR (Vα2.3/Vβ12.3) was truncated using PCR with the forward and reverse primers shown in FIG. 9. This TCR β-chain was cloned into the NdeI and XmaI sites of a pGMT7 vector (JMB002) containing the c-fos-BT fragment.

After construction of the fusion expression vectors, DNA sequencing was carried out to ensure no mistakes had been introduced during the sub-cloning procedure (all sequencing was carried out in the Biochemistry Dept. DNA Sequencing Facility, Oxford University using an ABI 377 Prism sequencer and ABI BIGDYE™ fluorescent terminators). It emerged that there were two errors in the tax TCR β-chain compared with the published sequence and upon further investigation, we discovered that these were both present in the original plasmid we had received. Since both of these errors were 3′ of a unique Bsu361 site in the TCR β-chain, this was used to clone into the (correct) JMB002 plasmid. Both versions of the tax TCR β-chain were expressed and refolded with α-chain and compared using Biacore. Both versions of the protein specifically bound to the tax peptide -MHC class I molecules with similar apparent affinities (see Example 17). In subsequent experiments, only the correct version of the β-chain was used.

### Example 9

### Expression of TCR Chains in E. coli and Purification of Inclusion Bodies

TCR α and β chains were expressed separately in the E. coli strain BL21 DE3pLysS under the control of the vector pGMT7 in TYP media, using 0.5 mM IPTG to induce protein production when the optical density (OD) at 600 nm reached between 0.2 and 0.6. Induction was allowed to continue overnight and the bacteria were harvested by centrifugation at 4000 rpm in a Beckman J-6B centrifuge.

Bacterial cell pellets were then resuspended in ‘lysis buffer’ (10 mM Tris pH 8.1, 10 mM EDTA, 150 mM NaCl, 2 mM DTT, 10% glycerol). The mixture was cooled on ice and the following were added: 20 μg/ml lysozyme, 10 mM MgCl₂, and 20 μg/ml DNase I, followed by incubation on ice for a minimum of an hour.

The mixture was then sonicated using a 12 mM probe sonicator (Milsonix XL2020) at full power for 5 bursts of 30 s with intervals of 30 s to allow the mixture to cool down. Temperature was maintained during this procedure by use of an ice-water mixture. The mixture was then diluted with 5 volumes of ‘Triton wash buffer’ (50 mM Tris pH 8.1, 0.5% Triton X-100, 100 mM NaCl, 0.1% sodium azide, 10 mM EDTA, 2 mM DTT). After incubation on ice for a minimum of 1 hour, the mixture was then centrifuged at 3,500 rpm in a Beckman GS-6R centrifuge and the supernatant was discarded. The pellet was resuspended in ‘Resuspension buffer’ (50 mM Tris pH 8.1, 100 mM NaCl, 10 mM EDTA, 2 mM DTT) using a small plastic disposable pipette. The mixture was then centrifuged at 8,000 rpm in a Beckman J2-21 centrifuge and the supernatant discarded. The pellet was then resuspended in ‘Guanidine buffer’ (50 mM Tris pH 8.1, 6.0 M Guanidine-HCl, 100 mM NaCl, 10 mM EDTA, 10 mM DTT) using a hand-operated homogenizer. After low-speed centrifugation to remove insoluble material, the supernatant was aliquotted and stored at −70° C. An approximate yield of 100 mg per litre of bacterial culture was routinely obtained.

SDS-PAGE analysis of the purified inclusion body preparation was achieved by diluting 2 μl of inclusion body preparation in Guanidine buffer with SDS-PAGE sample buffer followed by heating to 100° C. for 2 minutes. Samples were loaded onto the gel while still warm to prevent the Guanidine/SDS mixture from precipitating during loading. Inclusion body protein purified in this way was judged to be approximately 90% pure by Coomassie staining of SDS-PAGE performed in this way (see FIG. 23).

### Example 10

### Refolding and Purification of the TCRz Heterodimer

Urea-solublized proteins in equal proportions were further denatured in ‘guanidine buffer’ (6 M guanidine-HCl, 10 mM sodium acetate pH 5.5, 10 mM EDTA, 2 mM DTT) at 37° C. The mixture of proteins was injected into ice-cold refolding buffer (100 mM Tris pH 8.1, 0.4 M L-Arginine-HCl, 5.0 M Urea, 5 mM reduced glutathione, 0.5 mM oxidized glutathione) at a total protein concentration of 60 mg/L ensuring rapid mixing. After incubation on ice for at least 5 hours to allow refolding, the mixture was dialyzed against 10 volumes of demineralized water for 24 hours and then against 10 volumes of 10 mM Tris pH 8.1 for 24 hours.

The dialyzed refolded protein was then filtered to remove aggregated protein (produced as a by-product during the refolding) through a 0.45μ nitro-cellulose membrane (Whatman). Purification of the biotin-tagged soluble TCR was then performed by loading onto a POROS™ 20HQ column run on a BIOCAD™ Sprint system. Approximately 500 ml of refolded protein solution could be loaded per run and elution of the protein was achieved by a gradient of sodium chloride in Bis-Tris-Propane buffer pH 8.0. The protein eluted at approximately 100 mM sodium chloride and the relevant fractions were immediately chilled on ice and protease inhibitor cocktail was added. Fractions were analyzed by Coomassie-stained SDS-PAGE.

### Example 11

### Refolding and Purification of the TCRz Heterodimer with a Biotinylatable βchain.

Biotin-tagged TCR β-chains were mixed with an equal quantity of α-chain expressed and purified as for the soluble T cell receptor. Heterodimeric TCRz-β-BT was refolded according to identical procedures as described in Example 10 for TCRz (see FIG. 26).

### Example 12

### Biotinylation of Biotin-Tagged Soluble TCRz-BT

Protein-containing fractions were concentrated to 2.5 ml using 10 K-cut-off centrifugal concentrators (ULTRAFREE™, Millipore). Buffer was exchanged using PD-10 desalting columns equilibrated with 10 mM Tris pH 8.1, 5 mM NaCl, further protease inhibitor cocktail was added, and the protein was concentrated to ˜1 ml using centrifugal concentrators again. To this 1 ml of biotin-tagged soluble TCR the following were added: 7.5 mM MgCl₂, 5 M ATP (pH 8.0), 1 mM biotin, 2.5 μg/ml BirA biotinylation enzyme. The biotinylation reaction was then allowed to proceed at room temperature (20-25° C.) overnight.

Enzymatically biotinylated soluble TCR was then separated from residual unreacted biotin by gel filtration on a SUPERDEX™ 200 HR column (Pharmacia) run on a Pharmacia FPLC system (see FIG. 27). The column was equilibrated with PBS and 1 ml fractions were collected which were immediately chilled on ice and protected with protease inhibitor cocktail again. Protein concentration was estimated using a Coomassie-binding assay (Pierce) and the biotinylated protein was then stored at 4° C. for up to a month or at −20° C. for longer periods.

The efficacy of the biotinylation reaction was checked using Western blotting of the biotinylated protein. An SDS-PAGE gel was run using the methods described before, but instead of staining, the gel was blotted onto a PVDF membrane (Bio-Rad) using a SemiPhor semi-dry electoblotting apparatus (Hoefer). The blotting stack comprised of 6 layers of filter paper (Whatman 4M) cut to the size of the gel and soaked in transfer buffer (25 mM Tris base, 150 mM glycine) followed by the PVDF membrane which was pre-wetted with methanol and then soaked in transfer buffer, followed by the gel which was gently agitated in transfer buffer for 5 minutes, followed by 6 more layers of soaked filter paper. The stack was gently compressed using a test-tube to roll out any air-bubbles and approximately 10 ml of additional transfer buffer was added to aid conduction. The cathode was placed on top of the stack and current was passed through the apparatus at a constant current of 50 mA for 1 hour. The membrane was then incubated in a 2% solution of gelatin (Bio-Rad) in PBS-T buffer (PBS+0.05% Tween-20) for >1 hour at room temperature with gentle agitation. Overnight incubations also included 0.01% sodium azide to inhibit bacterial growth. The membrane was washed with several (4-5) changes of PBS-T followed by staining with avidin-HRP conjugate (Sigma) diluted 1:1000 in a 1% solution of gelatin in PBS-T for >30 minutes at room temperature with gentle agitation. The membrane was then washed with several (4-5) changes of PBS-T prior to detection with Opti-4CN (Bio-Rad). This is a reagent with reacts in the presence of HRP to form an insoluble blue dye which stains the membrane in the place where relevant protein is present as indicated by the presence of bound HRP. When avidin-HRP conjugate is used to stain, this therefore indicates the presence of a biotin-containing protein.

FIG. 28 shows a blot performed in such a way on several biotinylated TCRs. The standards run on this blot were biotinylated broad range molecular weight markers (Bio-Rad). The blot clearly shows that a high level of biotinylation of the TCRs containing the biotinylation tag which have been reacted with the BirA enzyme.

### Example 13

### Production of Biotinylated Soluble MHC-Peptide Complexes

Biotinylated soluble MHC-peptide complexes can be produced as described in Example 2.

### Example 14

### Assay for Specific Binding Between Soluble TCR and MHC-flu-peptide

The soluble TCR molecule, JM22z, is specific for HLA-A2 MHC molecules presenting an immuno dominant antigen consisting of amino acid residues 58-66 (GILGFVFTL) (SEQ ID NO. 79) of the influenza matrix protein. The cloning, expression, and purification of JM22z is described in Examples 4, 7, 9 and 10 and in FIGS. 24 and 25. The interactions between JM22z and its ligand/MHC complex (HLA-A2-flu) or an irrelevant HLA-A2 peptide combination, the production of which is described in Example 13, were analyzed on a Biacore 2000™ surface plasmon resonance (SPR) biosensor. SPR measures changes in refractive index expressed in response units (RU) near a sensor surface within a small flow cell, a principle that can be used to detect receptor ligand interactions and to analyze their affinity and kinetic parameters. The probe flow cells were prepared by immobilizing the individual HLA-A2-peptide complexes in separate flow cells via binding between the biotin cross linked onto O₂ m and streptavidin which had been chemically cross linked to the activated surface of the flow cells. The assay was then performed by passing JM22z over the surfaces of the different flow cells at a constant flow rate, measuring the SPR response in doing so. Initially, the specificity of the interaction was verified by passing 28 μM JM22z at a constant flow rate of 5 μl min⁻¹ over three different surfaces; one coated with 2800 RU of HLA-A2-flu, the second coated with 4200 RU of HLA-A2 folded with an irrelevant peptide from HIV reverse transcriptase (HLA-A2-pol: ILKEPVHGV) (SEQ ID NO. 83), and the third coated with 4300 RU of CD5 (FIG. 29a inset). Injections of soluble JM22z at constant flow rate and different concentrations over HLA-A2-pol were used to define the background resonance (FIG. 29a). The values of these control measurements were subtracted from the values obtained with HLA-A2-flu (FIG. 29b) and used to calculate binding affinities expressed as the dissociation constant, Kd (FIG. 29c). The Kd of JM22z and the relevant MHC molecule was determined to be 15±4 μM (n=7) at 37° C. and 6.6±2 μM (n=14) at 25° C. Determination using immobilized TCR in the probe flow cell and soluble MHC-peptide complex gave a similar Kd of 5.6±4 μM (n=3) at 25° C. The on-rate of the interaction was determined to be between 6.7×10⁴ and 6.9×10⁴ M⁻¹s⁻¹ at 37° C. while the off-rate was 1.1 s⁻¹ (Willcox, Gao et al. 1999).

### Example 15

### Assay for Specific Binding Between Soluble Murine TCR and Murine MHC H2-D^{b}-NP

In this experiment, we used a murine TCR, F5, specific for a peptide derived from the influenza virus nucleoprotein (aa.366-374: ASNENMDAM) (SEQ ID NO. 81) presented by the murine H2-D^{b} MHC molecule (H2-D^{b}-NP). The MHC heavy chain gene used was slightly modified in the sense that it encoded only amino acids 1-280 of the native protein plus a β-amino acid sequence recognized by the BirA enzyme. The resulting protein can be biotinylated enzymatically (Schatz 1993; O'Callaghan, Byford et al. 1999). SPR analysis on the Biacore 2000™ SPR biosensor using this soluble TCR specific for immobilized H2-D^{b}-NP showed that it bound specifically to the ligand MHC-peptide combination (data not shown).

### Example 16

### Comparison of Binding of Biotinylated Soluble Tax-TCR with Biotinylated Soluble Mutant tax-TCR

Biotinylated soluble tax-TCRs were prepared as in Examples 9-11 and Biacore 2000 analysis was performed as in Example 14 using biotinylated pMHC complexes refolded with either influenza matrix peptide (GILGFVFTL) (SEQ ID NO. 79) or HTLV tax 11-19 peptide (LLFGYPVYV) (SEQ ID NO. 82). Biotinylated soluble TCRs were flowed over all cells at 5 μl/minute for a total of 1 minute. FIG. 30 shows the binding of firstly the biotinylated soluble tax-TCR and then the biotinylated soluble mutant tax-TCR to HTLV tax 11-19 peptide-MHC complex (A). Neither the wild-type nor the mutant tax-TCR showed binding to either the influenza matrix peptide-MHC complex (B/C) or OX68 monoclonal antibody control (D). Therefore, we conclude that both the wild-type and the mutant biotinylated soluble TCRs clearly bind effectively and specifically to the tax-pMHC complex and show very little difference in the degree of binding.

### Example 17

### Analysis of Simultaneous TCR— and CD8 Co-Receptor Binding to Immobilized MHC Peptide Complex

CD8 and CD4 are surface glycoproteins believed to function as co-receptors for TCRs by binding simultaneously to the same MHC molecules as the TCR. CD8 is characteristic for cytotoxic T cells and binds to MHC class I molecules while CD4 is expressed on T cells of the helper lineage and binds MHC class 11 molecules. CID8 is a dimer consisting of either two identical α-chains or of an α- and a α-chain. The homodimeric αα-CD8 molecule was produced as described (PCT/GB98/03235; (Gao, Tormo et al. 1997; Gao, Gerth et al. 1998). In this example, we describe the simultaneous binding of soluble TCR and CD8 molecules to immobilized HLA-A2-flu complex. As seen in FIG. 31A, the binding response was simply additive. Subtracting the values of the TCR response (open circles) from the values of the combined response (closed circles) gave values (open squares) very close to the value of the response of 120 μM CD8 alone (open triangles). FIG. 31B shows that the kinetics of the TCR-MHC-peptide interaction was unaffected by simultaneous CD8 binding. The observed additive biding indicate that TCR and CD8 bind the MHC peptide complex at separate interfaces. The example also illustrates that in some cases specific binding of one molecule will not influence specific binding of another molecule, a situation most likely to be different for other combinations of molecules.

### Example 18

### Expression, Refolding and Site-Specific Biotinylation of Soluble α/β TCR

a) Engineering of TCR α and β Chains.

A recombinant soluble form of the heterodimeric TCR molecule was engineered as outlined in FIG. 36. Each chain consists of membrane-distal and -proximal immunoglobulin domains which are fused via a short flexible linker to a coiled coil motif which helps stabilize the heterodimer.

FIGS. 32 to 34 and 38 to 41 show the DNA coding sequences and corresponding amino acid sequences for various TCR alpha and beta chains from TCR having different specificities. This example concentrates on the TCR represented by the sequences of FIGS. 32 to 34 but the methods disclosed can be similarly performed using the TCRs given in FIGS. 38 to 41.

The TCR constant domains have been truncated immediately before cysteine residues which in vivo form an interchain disulfide bond. Consequently the two chains pair by non-covalent quaternary contacts. As the Fos-Jun zipper peptide heterodimers are also capable of forming an interchain disulfide immediately N-terminal to the linker used (O'Shea et al. 1989), the alignment of the two chains relative to each other was predicted to be optimal. Fusion proteins need to be joined in a manner which is compatible with each of the separate components, in order to avoid disturbing either structure.

cDNA encoding alpha and beta chains of a TCR specific for the influenza-matrix protein 58-66 epitope in HLA-A2 was obtained from a Vβ17+human CTL clone (JM22) by anchored PCR as described previously (Moss et al. 1991).

Alpha and beta TCR-zipper constructs pJM22α-Jun and pJM22α-Fos were separately constructed by amplifying the variable and constant domain of each chain using standard PCR technology and splicing products onto leucine zipper domains from the eucaryotic transcription factors Jun and Fos respectively. These 40 amino acid long sequences have been shown to specifically heterodimerize when refolded from synthetic peptides, without the need for a covalent interchain linkage (O'Shea et al. 1989).

Primers were designed to incorporate a high AT content immediately 3′ to the initiation codon (to destabilize mRNA secondary structure) and using E. coli codon preferences, in order to maximize expression (Gao et al. 1998). The spare cysteine in the TCR beta constant domain was mutated to serine to ensure prevention of incorrect disulfide bonding during refolding.

The fused DNA and protein sequences are indicated in FIGS. 32 and 33. In order to enable the site-specific biotinylation of the β chain of this TCR a DNA sequence encoding a so-called “biotin-tag” was engineered into the 3′ end of the gene expressing soluble Vβ17. The following PCR primers were employed for the engineering of this DNA construct:

5′-GCTCTAGACATATGGGCCCAGTGGATTCTGGAGTCAC-3′ (SEQ ID NO. 84) and

(SEQ ID NO. 85)

5′-GGGGGAAGCTTAATGCCATTCGATTTTCTGAGCTTCAAAAATATCGT
TCAGACCACCACCGGATCCGTAAGCTGCCAGGATGAACTCTAG-3′.

The resulting PCR product was digested with restriction enzymes NdeI and HindIII (New England Biolabs) and ligated with T4 DNA ligase (New England Biolabs) into the vector pGMT7 (Studier et al. 1990). FIG. 3 shows the DNA sequence of the insert in this construct and the deduced protein sequence.

b) Expression of TCR Chains.

Expression and refolding of a TCR with specificity for the Influenza virus Matrix peptide presented by HLA-A*0201 was carried out as follows:

TCR α and β chains were expressed separately in the E. coli strain BL21 DE3pLysS under the control of the vector pGMT7 in TYP media (1.6% bacto-tryptone, 1.6% yeast extract, 0.5% NaCl, 0.25% K2HPO4). Expression was induced in mid-log phase with 0.5 mM IPTG and, after 3-5 hours, bacteria were harvested by centrifugation. The bacterial cells were lysed by resuspension in ‘lysis buffer’ (10 mM EDTA, 2 mM DTT, 10 mM Tris pH 8, 150 mM NaCl, 0.5 mM PMSF, 0.1 mg/ml lysozyme, 10% glycerol) followed by addition of 10 mM MgCl2 and 20 ug/ml DNasel, incubation for 20 minutes on ice, and sonication using a probe sonicator in 10× bursts of 30 seconds. The protein, in inclusion bodies, was then purified by several washes (usually 3) of ‘Triton buffer’ (0.5% Triton X-100, 50 mM Tris pH8, 100 mM NaCl, 0.1% sodium azide, 10 mM EDTA, 2 mM DTT) using centrifugation at 15,000 rpm for 20 minutes to pellet the inclusion bodies and a ‘dounce’ homogeniser to resuspend them. Detergent was removed from the preparation with a single wash of 50 mM Tris pH 8, 100 mM NaCl, 10 mM EDTA, 2 mM DTT and the protein was solubilized with ‘urea buffer (20 mM Tris pH 8, 8 M urea, 10% glycerol, 500 mM NaCl, 10 mM EDTA, 2 mM DTT). After end-over-end mixing overnight at 4° C., the solution was clarified by centrifugation, and the solubilized protein was stored at −70° C. The protein concentration was measured by a Coomassie-binding assay (Pierce).

c) Refolding of the TCR.

Urea-solublized protein in equal proportions was further denatured in ‘guanidine buffer’ (6 M guanidine-HCl, 10 mM sodium acetate pH 5.5, 10 mM EDTA, 2 mM DTT) at 37 C. This solution was added to refolding buffer (5 M urea, 100 mM Tris pH 8, 400 mM L-arginine, 5 mM reduced glutathione, 0.5 mM oxidized glutathione, 0.1 mM PMSF) on ice ensuring rapid mixing. After >12 hours at 4° C., the solution was dialyzed against 10 volumes of water, then 10 volumes of 10 mM Tris pH 8, 100 mM urea. The protease inhibitor PMSF was added at all stages to minimize proteolytic loss of the biotinylation tag on the TCR.

d) Purification of the TCR.

The dilute solution of the TCR was filtered through a 0.45 micron filter to remove aggregated protein and was then loaded onto a POROS™ 10HQ column. The refolded TCR was eluted with a gradient of sodium chloride in 10 mM Tris pH 8 and 1 ml fractions were collected and analyzed by SDS-PAGE (see FIG. 36). Fractions containing TCR were pooled and concentrated to 1 ml using a 30 kDa cut-off centrifugal concentrator.

e) Biotinylation of the TCR.

The 1 ml of TCR solution was made up to 7.5 mM ATP using buffered ATP, 5 mM MgCl2, 1 mM biotin, and a cocktail of protease inhibitors was added which included PMSF, leupeptin, and pepstatin. Finally, the enzyme BirA was added to a final concentration of 5 ug/ml and the reaction was allowed to proceed overnight at room temperature. The TCR was then separated from free biotin by gel filtration (see FIG. 37). Fractions containing biotinylated TCR were pooled and protease inhibitor cocktail was added. Protein concentration was also determined. FIG. 35 shows a schematic diagram of the soluble, biotinylated TCR.

### REFERENCES

Aifantis, I., O. Azogui, et al. (1998). Immunity 9(5): 649-55.
Altamirano, M. M., C. Garcia, et al. (1999). Nature Biotechnology 17: 187-191.
Altamirano, M. M., R. Golbik, et al. (1997). Proc Natl Acad Sci USA 94(8): 3576-8.
Amati, B., S. Dalton, et al. (1992). Nature 359(6394): 423-6.
Barker, D. F. and A. M. Campbell (1981). J Mol Biol 146(4): 469-92.
Barker, D. F., and Campbell, A. M. (1981). J Mol Biol 146, 469-92.
Bentley, G. A., G. Boulot, et al. (1995). Science 267 (5206): 1984-7 Issn: 0036-8075.
Bjorkman, P. J., J. L. Strominger, et al. (1985). J. Mol. Biol 186(1): 205-10 Issn: 0022-2836
Boice, J. A., G. R. Dieckmann, et al. (1996). Biochemistry 35(46): 14480-5.
Boulot, G., G. A. Bentley, et al. (1994). J Mol Biol 235(2): 795-7 Issn: 0022-2836.
Brocker, T., A. Peter, et al. (1993). Eur J Immunol 23(7): 1435-9 Issn: 0014-2980.
Buday, L. and J. Downward (1993). Cell 73(3): 611-20.
Calaman, S. D., G. R. Carson, et al. (1993). J Immunol Methods 164(2): 233-44 Issn: 0022-1759.
Chao, H., M. E. Houston, Jr., et al. (1996). Biochemistry 35(37): 12175-85.
Chang, H. C., Z. Bao, et al. (1994). Proc Natl Acad Sci USA 91(24): 11408-12.
Chevray, P. M. and D. Nathans (1992). Proc Natl Acad Sci USA 89(13): 5789-93.
Chicz, R. M., R. G. Urban, et al. (1993). J Exp Med 178(1): 27-47.
Corr, M., A. E. Slanetz, et al. (1994). Science 265(5174): 946-9.
Davis, M. M., J. J. Boniface, et al. (1998). Annu. Rev. Immunol. 16: 523-544.
de Kruif, J. and T. Logtenberg (1996). J Biol Chem 271(13): 7630-4.
Ding, Y. H., K. J. Smith, et al. (1998). Immunity 8(4): 403-11.
Eilat, D., G. E. Kikuchi, et al. (1992). Proc Natl Acad Sci USA 89(15): 6871-5 Issn: 0027-8424.
Engelhard, V. H. (1994). Annu Rev Immunol 12:181-207.
Engelhard, V. H., E. Appella, et al. (1993). Chem Immunol 57: 39-62.
Fields, B. A., E. L. Malchiodi, et al. (1996). Nature 384(6605): 188-92 Issn: 0028-0836.
Gao, G. F., U. C. Gerth, et al. (1998). Prot. Sci. 7: 1245-49.
Gao, G. F., J. Tormo, et al. (1997). Nature 387(6633): 630-4.
Garboczi, D. N. and W. E. Biddison (1999). Immunity 10(1): 1-7.
Garboczi, D. N., P. Ghosh, et al. (1996). Nature 384(6605): 134-41.
Garboczi, D. N., D. T. Hung, et al. (1992). Proc Natl Acad Sci USA 89(8): 3429-33 Issn: 0027-8424.
Garboczi, D. N., D. R. Madden, et al. (1994). J Mol Biol 239(4): 581-7 Issn: 0022-2836.
Garboczi, D. N., U. Utz, et al. (1996). J Immunol 157(12): 5403-10.
Garcia, K. C., M. Degano, et al. (1996). Science 274(5285): 209-19 Issn: 0036-8075.
Garcia, K. C., C. A. Scott, et al. (1996). Nature 384(6609): 577-81 Issn: 0028-0836.
Glover, J. N. and S. C. Harrison (1995). Nature 373(6511): 257-61.
Golden, A., S. S. Khandekar, et al. (1997). J Immunol Methods 206(1-2): 163-9.
Greenfield, N. J., G. T. Montelione, et al. (1998). Biochemistry 37(21): 7834-43.
Gregoire, C., B. Malissen, et al. (1996). Eur J Immunol 26(10): 2410-6 Issn: 0014-2980.
Gregoire, C., N. Rebai, et al. (1991). Proc Natl Acad Sci USA 88(18): 8077-81 Issn: 0027-8424.
Hilyard et al (1994) Proc. Natl. Acad. Sci. 91: 9057-9061.
Howard, P. K., J. Shaw, et al. (1985). Gene 35(3): 321-31.
Hu, J. C., N. E. Newell, et al. (1993). Protein Sci 2(7): 1072-84.
Huczko, E. L., W. M. Bodnar, et al. (1993). J Immunol 151(5): 2572-87.
Hunt, D. F., H. Michel, et al. (1992). Science 256(5065): 1817-20 Issn: 0036-8075.
Ishii, Y., T. Nakano, et al. (1995). J Immunol Methods 186(1): 27-36 Issn: 0022-1759.
Kouzarides, T. and E. Ziff (1989). Nature 340(6234): 568-71.
Landschulz, W. H., P. F. Johnson, et al. (1988). Science 240(4860): 1759-64.
Lowenstein, E. J., R. J. Daly, et al. (1992). Cell 70(3): 431-42.
Lumb, K. J. and P. S. Kim (1995). Biochemistry 34(27): 8642-8.
Madden, D. R., D. N. Garboczi, et al. (1993). [published erratum appears in Cell 1994 Jan. 28;76(2):following 410].” Cell 75(4): 693-708 Issn: 0092-8674.
Matsui, K., J. J. Boniface, et al. (1994). Proc Natl Acad Sci USA 91 (26): 12862-6 Issn: 0027-8424.
McKnight, S. L. (1991). Sci Am 264(4): 54-64.
Moss et al. (11991) Proc. Natl. Acad. Sci. USA 88: 8987-8990 Issn: 0027-8424.
Nautiyal, S., D. N. Woolfson, et al. (1995). Biochemistry 34(37): 11645-51.
Necker, A., N. Rebai, et al. (1991). Eur J Immunol 21(12): 3035-40 Issn: 0014-2980.
O'Callaghan, C. A., M. F. Byford, et al. (1999). Anal Biochem 266(1): 9-15.
O'Shea, E. K., R. Rutkowski, et al. (1992). Cell 68(4): 699-708.
O'Shea, E. K., R. Rutkowski, et al. (1989). Science 245(4918): 646-8.
O'Shea, E. K., Lumb, K. J. and Kim, P. S. (1993) Curr. Biol. 3: 658-667.
Plaksin, D., K. Polakova, et al. (1997). J Immunol 158(5): 2218-27.
Rabinowitz, J. D., C. Beeson, et al. (1996). Proc Natl Acad Sci USA 93(4): 1401-5 Issn: 0027-8424.
Ramiro, A. R., C. Trigueros, et al. (1996). J Exp Med 184(2): 519-30 Issn: 0022-1007.
Reid, S. W., S. McAdam, et al. (1996). J Exp Med 184(6): 2279-86.
Reid, S. W., K. J. Smith, et al. (1996). FEBS Left 383(1-2): 119-23.
Riley, L. G., G. B. Ralston, et al. (1996). [published erratum appears in Protein Eng 1996 September;9(9):831].” Protein Eng 9(2): 223-30.
Romagne, F., M. A. Peyrat, et al. (1996). J Immunol Methods 189(1): 25-36 Issn: 0022-1759.
Saint Ruf C., K. Ungewiss, et al. (1994). Science 266(5188): 1208-12 Issn: 0036-8075.
Schatz, P. J. (1993). Biotechnology NY 11(10): 1138-43.
Schlessinger, J. (1994). Curr Opin Genet Dev 4(1): 25-30.
Schlueter, C. J., B. A. Schodin, et al. (1996). J Mol Biol 256(5): 859-69.
Schuermann, M., J. B. Hunter, et al. (1991). Nucleic Acids Res 19(4): 739-46.
Smith, K. J., S. W. Reid, et al. (1996). Immunity 4(3): 215-28 Issn: 1074-7613.
Smith, K. J., S. W. Reid, et al. (1996). Immunity 4(3): 203-13 Issn: 1074-7613.
Studier, F. W., A. H. Rosenberg, et al. (1990). Methods Enzymol 185: 60-89 Issn: 0076-6879.
von Boehmer, H., I. Aifantis, et al. (1998). Immunol Rev 165:111-9.
Weber, S., A. Traunecker, et al. (1992). Nature 356(6372): 793-6 Issn: 0028-0836.
Willcox, B. E., G. F. Gao, et al. (1999). Immunity 10: 357-65.
Wilson, A. and H. R. MacDonald (1995). Int Immunol 7(10): 1659-64 Issn: 0953-8178.
Wurch, A., J. Biro, et al. (1998). J Exp Med 188(9): 1669-78.
Wyer, J. R., B. E. Willcox, et al. (1999). Immunity 10: 219-225.
Zhang, Z., A. Murphy, et al. (1999). Curr Biol 9(8): 417-20.

1. A recombinant soluble T cell receptor (TCR) which comprises:
i) a TCR α or γ chain extracellular domain which comprises a variable domain and a constant domain, and which has a first C-terminal dimerization peptide which is heterologous to the α or γ chain; and
ii) a TCR β or δ chain extracellular domain which comprises a variable domain and a constant domain, and which has a second C-terminal dimerization peptide which is heterologous to the β or δ chain;
wherein the first dimerization peptide and the second dimerization peptide are specifically heterodimerized to form a heterodimerization domain;
wherein a disulfide bond present in native TCRs between the α and β or γ and δ chain is absent; and
wherein the TCR is capable of specific binding to a peptide-MHC complex at a concentration of at least 40 μg/ml.

2. The recombinant TCR according to claim 1, wherein said TCR is stable at low concentrations.

3. The recombinant TCR according to claim 1, wherein said TCR is stable at a concentration of about 20 mg/ml.

4. The recombinant TCR according to claim 1, wherein said TCR is stable at a concentration below 1 mg/ml.

5. The recombinant TCR according to claim 1, wherein said TCR is stable at a concentration of about 10 μg/ml.

6. The recombinant TCR according to claim 1, wherein the heterodimerization domain is a coiled coil domain.

7. The recombinant TCR according to claim 6, wherein the dimerization peptides are c-jun and c-fos dimerization peptides.

8. The recombinant TCR according to claim 1, comprising a flexible linker located between the TCR chains and the dimerization peptides.

9. The recombinant TCR according to claim 1, expressed in an E. coli expression system.

10. The recombinant TCR according to claim 1, which is biotinylated at the C-terminus.

11. The recombinant TCR according to claim 1, labeled with a detectable label.

12. The recombinant TCR according to claim 1, linked to a therapeutic agent.

13. A recombinant non-membrane-bound T cell receptor produced by:
i) expressing a TCR α or γ chain extracellular domain which comprises a variable domain and a constant domain, and which has a first C-terminal dimerization peptide which is heterologous to the α or γ chain;
ii) expressing a TCR β or δ chain extracellular domain which comprises a variable domain and a constant domain, and which has a second C-terminal dimerization peptide which is heterologous to the α or γ chain; and
iii) refolding the chains together in vitro to produce a TCR heterodimer;
wherein the first and second dimerization peptides form a heterodimerization domain;
wherein a disulfide bond present in native TCRs between the α and β or γ and 6 chains is not formed; and
wherein the TCR is capable of specific binding to a peptide-MHC complex at a concentration of at least 40 μg/ml.

14. The recombinant TCR according to claim 12, wherein the therapeutic agent is an immunostimulatory agent.


SEQUENCE LISTING (85 sequences)

SEQ ID NO: 1 - DNA, 33 bp, Artificial Sequence
  Description of Artificial Sequence Forward poly-C“anchor” primer for PCR-amplificaton of cDNAs extended at their 3′-terminal with a stretch of G-residues using Terminal transferase. (Figure 4A)
taaatactcgaggcgcgcccccccccccccccc

SEQ ID NO: 2 - DNA, 48 bp, Artificial Sequence
  Description of Artificial SequenceHuman TCR alpha chain constant region 3′-specific primer. (Figure 4B).
atataacccggggaaccagatccccacaggaactttctgggctgggga

SEQ ID NO: 3 - DNA, 47 bp, Artificial Sequence
  Description of Artificial Sequence Human TCR beta chain constant region 3′-specific PCR primer.
atataacccggggaaccagatccccacagtctgctctaccccaggcc

SEQ ID NO: 4 - DNA, 33 bp, Artificial Sequence
  Description of Artificial Sequence Human c-jun leucine zipper 5′-specific PCR primer.
catacacccgggggtagaatcgcccggctggag

SEQ ID NO: 5 - DNA, 50 bp, Artificial Sequence
  Description of Artificial Sequence Human c-jun leucine zipper 3′-specific PCR primer. (Figure 5B).
gtgtgtgctcgaggatcctagtagttcatgactttctgtttaagctgtgc

SEQ ID NO: 6 - DNA, 39 bp, Artificial Sequence
  Description of Artificial Sequence Human c-fos leucine zipper 5′-specific PCR primer. (Figure 5C).
catacacccgggggtctgactgatacactccaagcggag

SEQ ID NO: 7 - DNA, 49 bp, Artificial Sequence
  Description of Artificial Sequence Human c-fos leucine zipper 3′-specific PCR primer. (Figure 5D).
tgtgtgctcgaggatcctagtaagctgccaggatgaactctagtttttc

SEQ ID NO: 8 - DNA, 120 bp, Homo sapiens
  Partial human c-fos sequence coding for the leucine zipper domain as fused to TCR beta chains. (Figure 6B).
ctgactgatacactccaagcggagacagaccaactagaagatgagaagtctgctttgcagaccgagattgccaacctgctgaaggagaaggaaaaactagagttcatcctggcagcttac

SEQ ID NO: 9 - DNA, 120 bp, Homo sapiens
  Partial human c-jun sequence coding for the leucine zipper domain as fused to TCR alpha chains. (Figure 6A).
agaatcgcccggctggaggaaaaagtgaaaaccttgaaagctcagaactcggagctggcgtccacggccaacatgctcagggaacaggtggcacagcttaaacagaaagtcatgaactac

SEQ ID NO: 10 - protein, 40 aa, Homo sapiens
  C-jun leucine zipper amino acid sequence as fused to TCR alfa chains. (Figure 6A)
RIARLEEKVKTLKAQNSELASTANMLREQVAQLKQKVMNY

SEQ ID NO: 11 - protein, 40 aa, Artificial Sequence
  C-fos leucine zipper amino acid sequence as fused to TCR beta chains. (Figure 6B).
LTDTLQAETDQLEDEKSALQTEIANLLKEKEKLEFILAAY

SEQ ID NO: 12 - DNA, 26 bp, Artificial Sequence
  Description of Artificial Sequence Forward PCR primer for mutating the unpaired cysteine of human TCR beta chains to serine (Figure 7A).
gactccagatacagcctgagcagccg

SEQ ID NO: 13 - protein, 8 aa, Artificial Sequence
  Amino acid sequence of the human TCR beta chain after mutating the unpaired cysteine to serine (Figure 7A).
DSRYSLSS

SEQ ID NO: 14 - DNA, 26 bp, Artificial Sequence
  Description of Artificial SequenceBackward PCR primer for mutating the unpaired cysteine of human TCR beta chains to serine (Figure 7B).
cggctgctcaggctgtatctggagtc

SEQ ID NO: 15 - DNA, 26 bp, Artificial Sequence
  Description of Artificial Sequence Forward PCR primer for mutating the unpaired cysteine of human TCR beta chains to alanine (Figure 7C).
gactccagatacgctctgagcagccg

SEQ ID NO: 16 - protein, 8 aa, Artificial Sequence
  Description of Artificial Sequence Amino acid sequence of the human TCR beta chain after mutating the unpaired cysteine to alanine (Figure 7C).
DSRYALSS

SEQ ID NO: 17 - DNA, 26 bp, Artificial Sequence
  Description of Artificial Sequence Backward PCR primer for mutating the unpaired cysteine of human TCR beta chains to alanine (Figure 7D).
cggctgctcagagcgtatctggagtc

SEQ ID NO: 18 - DNA, 57 bp, Artificial Sequence
  Description of Artificial Sequence 5′ PCR primer for the human V alpha10.2 chain of the JM22 Influenza matrix peptide-HLA-A0201 restricted TCR. (Figure 9A).
gctctagacatatgcaactactagaacaaagtcctcagtttctaagcatccaagagg

SEQ ID NO: 19 - protein, 15 aa, Homo sapiens
  New N-terminal amino acid sequence of truncated V alpha10.2 chain of the human JM22 Influenza matrix peptide-HLA-A0201 restricted TCR. (Figure 9A).
MQLLEQSPQFLSIQE

SEQ ID NO: 20 - DNA, 39 bp, Artificial Sequence
  Description of Artificial Sequence 5′ PCR primer for the human V beta17 chain of the JM22 Influenza matrix peptide-HLA-A0201 restricted TCR. (Figure 9B)
gctctagacatatggtggatggtggaatcactcagtccc

SEQ ID NO: 21 - protein, 9 aa, Artificial Sequence
  New N-terminal amino acid sequence of truncated V beta17 chain of the human JM22 Influenza matrix peptide-HLA-A0201 restricted TCR. (Figure 9B).
MVDGGITQS

SEQ ID NO: 22 - DNA, 57 bp, Artificial Sequence
  Description of Artificial Sequence 5′ PCR primer for the mouse V alpha4 chain of the Influenza nucleoprotein peptide-H2Db restricted TCR. (Figure 9C).
gctctagacatatggattctgttactcaaatgcaaggtcaagtgaccctctcatcag

SEQ ID NO: 23 - protein, 15 aa, Artificial Sequence
  Description of Artificial Sequence New N-terminal amino acid sequence of truncated V alpha4 chain of the mouse  Influenza nucleoprotein peptide-H2-Db restricted TCR. (Figure 9C).
MDSVTQMQGQVTLSS

SEQ ID NO: 24 - DNA, 53 bp, Mus musculus
  5′ PCR primer for the mouse V beta 11 chain of the Influenza nucleoprotein peptide-H2Db restricted TCR. (Figure 9D).
gctctagacatatggaaccaacaaatgctggtgttatccaaacacctaggcac

SEQ ID NO: 25 - protein, 14 aa, Mus musculus
  New N-terminal amino acid sequence of truncated murine V beta 11 chain of the Influenza nucleoprotein peptide-H2-Db restricted TCR .
MEPTNAGVIQTPRH

SEQ ID NO: 26 - DNA, 36 bp, Homo sapiens
  5′ PCR primer for the human V alpha 23 chain of the 003 HIV-1 GAGag peptide-HLA-A0201 restricted TCR. (Figure 9E).
ggaattccatatgaaacaagaggttacacaaattcc

SEQ ID NO: 27 - protein, 8 aa, Homo sapiens
  New N-terminal amino acid sequence of truncated human Valpha23 chain of the 003 HIV-1 Gag peptide-HLA-A0201 restricted TCR . (Figure 9E).
MKQEVTQI

SEQ ID NO: 28 - DNA, 36 bp, Homo sapiens
  5′ PCR primer for the human Vbeta5.1 chain of the 003 HIV-1 Gag peptide-HLA-A0201 restricted TCR. (Figure F).
ggaattccatatgaaagctggagttactcaaactcc

SEQ ID NO: 29 - protein, 8 aa, Homo sapiens
  New N-terminal amino acid sequence of truncated human Vbeta5.1 chain of the 003 HIV-1 Gag peptide-HLA-A0201 restricted TCR . (Figure 9F).
MKAGVTQT

SEQ ID NO: 30 - DNA, 33 bp, Homo sapiens
  5′ PCR primer for the human Valpha2.3 chain of the HTLV-1 Tax peptide-HLA-A0201 restricted A6 TCR. (Figure 9g).
cccccccatatgcagaaggaagtggagcagaac

SEQ ID NO: 31 - protein, 8 aa, Homo sapiens
  New N-terminal amino acid sequence of truncated human V alpha 2.3 chain of the HTLV-1 Tax peptide-HLA-A0201 restricted A6 TCR . (Figure 9G).
MQKEVEQK

SEQ ID NO: 32 - DNA, 33 bp, Homo sapiens
  5′ PCR primer for the human Vbeta12.3 chain of the HTLV-1 Tax peptide-HLA-A0201 restricted A6 TCR. (Figure 9H).
cccccccatatgaacgctggtgtcactcagacc

SEQ ID NO: 33 - protein, 8 aa, Homo sapiens
  New N-terminal amino acid sequence of truncated human Vbeta12.3 chain of the HTLV-1 Tax peptide-HLA-A0201 restricted A6 TCR . (Figure 9H).
MKAGVTQT

SEQ ID NO: 34 - DNA, 48 bp, Homo sapiens
  5′ PRC
cccccccatatgcaacaaaaaaatgatgaccagcaagttaagcaaaat

SEQ ID NO: 35 - protein, 13 aa, Homo sapiens
  New N-terminal amino acid sequence of truncated human Valpha17.2 chain of the HTLV-1 Tax peptide-HLA-A0201 restricted B7 TCR . (Figure 9I).
MQQKNDDQQVKQN

SEQ ID NO: 36 - DNA, 45 bp, Homo sapiens
  5′ PCR primer for the human Vbeta12.3 chain of the HTLV-1 Tax peptide-HLA-A0201 restricted B7 TCR. (Figure 9J)
cccccccatatgaacgctggtgtcactcagaccccaaaattccag

SEQ ID NO: 37 - protein, 12 aa, Homo sapiens
  New N-terminal amino acid sequence of truncated human Vbeta12.3 chain of the HTLV-1 Tax peptide-HLA-A0201 restricted B7 TCR . (Figure 9J).
MNAGVTQTPKFQ

SEQ ID NO: 38 - DNA, 38 bp, Homo sapiens
  3′ PCR primer for human Calpha chains, generally applicable.  (Figure 9K).
catacacccgggggaactttctgggctggggaagaagg

SEQ ID NO: 39 - DNA, 33 bp, Homo sapiens
  3′ PCR primer for human Cbeta chains, generally applicable. (Figure 9L).
catacacccggggtctgctctaccccaggcctc

SEQ ID NO: 40 - DNA, 744 bp, Homo sapiens
  Mutated DNA sequence of soluble HLA-A2/flu matrix restricted TCR alpha chain from JM22, as fused to the “leucine zipper” domain of human c-jun. (Figure 10).
atgcaactactagaacaaagtcctcagtttctaagcatccaagagggagaaaatctcactgtgtactgcaactcctcaagtgttttttccagcttacaatggtacagacaggagcctggggaaggtcctgtcctcctggtgacagtagttacgggtggagaagtgaagaagctgaagagactaacctttcagtttggtgatgcaagaaaggacagttctctccacatcactgcggcccagcctggtgatacaggcctctacctctgtgcaggagcgggaagccaaggaaatctcatctttggaaaaggcactaaactctctgttaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcccccgggggtagaatcgcccggctggaggaaaaagtgaaaaccttgaaagctcagaactcggagctggcgtccacggccaacatgctcagggaacaggtggcacagcttaaacagaaagtcatgaactactag

SEQ ID NO: 41 - protein, 247 aa, Homo sapiens
  Predicted amino acid sequence of the soluble HLA-A2/flu matrix restricted alpha chain from JM22, as fused to the “lecine zipper” domain of c-jun. (Figure 10).
MQLLEQSPQFLSIQEGENLTVYCNSSSVFSSLQWYRQEPGEGPVLLVTVVTGGEVKKLKRLTFQFGDARKDSSLHITAAQPGDTGLYLCAGAGSQGNLIFGKGTKLSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSPGGRIARLEEKVKTLKAQNSELASTANMLREQVAQLKQKVMNY

SEQ ID NO: 42 - DNA, 864 bp, Homo sapiens
  DNA sequence of the soluble HLA-A2/flu restricted TCR beta chain from JM22, as fused to the “leucine zipper” domain of c-fos.  (Figure 11).
atggtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagttcgaggagctcctacgagcagtacttcgggccgggcaccaggctcacggtcacagaggacctgaaaaacgttttcccacccgaggtcgctgtgtttgaaccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagaccccgggggtctgactgatacactccaagcggagacagatcaacttgaagacaagaagtctgcgttgcagaccgagattgccaatctactgaaagagaaggaaaaactagagttcatcctggcagcttactag

SEQ ID NO: 43 - protein, 287 aa, Homo sapiens
  Predicted amino acid sequence of the soluble HLA-A2/flu matrix restricted beta chain from JM22, as fused to the “lecine zipper” domain of c-fos. (Figure 11).
MVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADPGGLTDTLQAETDQLEDKKSALQTEIANLLKEKEKLEFILAAY

SEQ ID NO: 44 - DNA, 795 bp, Artificial Sequence
  DNA sequence of the soluble H2-Db/Influenza virus nucleoprotein restricted TCR alpha chain from murine F5 receptor, as fused to the “leucine zipper” domain of c-jun.  (Figure 12).
atgaactattctccagctttagtgactgtgatgctgtttgtgtttgggaggacccatggagactcagtaacccagatgcaaggtcaagtgaccctctcagaagacgacttcctatttataaactgtacttattcaaccacatggtacccgactcttttctggtatgtccaatatcctggagaaggtccacagctccttttgaaagtcacaacagccaacaacaagggaatcagcagaggttttgaagctacatatgataaaggaacaacgtccttccacttgcagaaagcctcagtgcaggagtcagactctgctgtgtactactgtgtgctgggtgatcgacagggaggcagagctctgatatttggaacaggaaccacggtatcagtcagccccaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatctccaaagagaccaacgccacctaccccagttcagacgttcccgggggtagaatcgcccggctggaggaaaaagtgaaaaccttgaaagctcagaactcggagctggcgtccacggccaacatgctcagggaacaggtggcacagcttaaacagaaagtcatgaactactag

SEQ ID NO: 45 - protein, 264 aa, Artificial Sequence
  Predicted amino acid sequence of the soluble H2-Db/Influenza virus nucleoprotein restricted alpha chain from murine F5 receptor, as fused to the “lecine zipper” domain of c-jun. (Figure 12).
MNYSPALVTVMLFVFGRTHGDSVTQMQGQVTLSEDDFLFINCTYSTTWYPTLFWYVQYPGEGPQLLLKVTTANNKGISRGFEATYDKGTTSFHLQKASVQESDSAVYYCVLGDRQGGRALIFGTGTTVSVSPNIQNPEPAVYQLKDPRSQDSTLCLFTDFDSQINVPKTMESGTFITDKTVLDMKAMDSKSNGAIAWSNQTSFTCQDISKETNATYPSSDVPGGRIARLEEKVKTLKAQNSELASTANMLREQVAQLKQKVMNY

SEQ ID NO: 46 - DNA, 864 bp, Artificial Sequence
  DNA sequence of soluble H2-Db/Influenza virus nucleoprotein restricted TCR beta chain from the murine F5 receptor, as fused to the leucine zipper domain of c-fos.(Figure 13).
atgaaagctggagttactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttcgacagcgggaattcacccctccactttgggaacgggaccaggctcactgtgacagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagccaggacccgcagcccctcaaggagcagcccgccctcaatgactccagatacagcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagaccccgggggtctgactgatacactccaagcggagacagatcaacttgaagacaagaagtctgcgttgcagaccgagattgccaatctactgaaagagaaggaaaaactagagttcatcctggcagcttactag

SEQ ID NO: 47 - protein, 287 aa, Artificial Sequence
  Amino acid sequence of soluble H2-Db/Influenza virus nucleoprotein restricted TCR beta chain from the murine F5 receptor, as fused to the leucine zipper domain of c-fos. (Figure 13).
MKAGVTQTPRYLIKTRGQQVTLSCSPISGHRSVSWYQQTPGQGLQFLFEYFSETQRNKGNFPGRFSGRQFSNSRSEMNVSTLELGDSALYLCASSFDSGNSPLHFGNGTRLTVTEDLNKVFPPEVAVFEPSEAEISHTQKATLVCLATGFFPDHVELSWWVNGKEVHSGVSQDPQPLKEQPALNDSRYSLSSRLRVSATFWQNPRNHFRCQVQFYGLSENDEWTQDRAKPVTQIVSAEAWGRADPGGLTDTLQAETDQLEDKKSALQTEIANLLKEKEKLEFILAAY

SEQ ID NO: 48 - DNA, 747 bp, Artificial Sequence
  DNA sequence of soluble HLA-A2/HIV-1 Gag restricted TCRalpha chain from patient 003r, as fused to the leucine zipper domain of c-jun. (Figure 14).
atgaaacaagaagttacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaccaacttcaacaaattttactttggatctgggaccaaactcaatgtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcccccgggggtagaatcgcccggctggaggaaaaagtgaaaaccttgaaagctcagaactcggagctggcgtccacggccaacatgctcagggaacaggtggcacagcttaaacagaaagtcatgaactactag

SEQ ID NO: 49 - protein, 248 aa, Artificial Sequence
  Description of Artificial Sequence Amino acid sequence of soluble HLA-A2/HIV-1 Gag restricted TCRalpha chain from patient 003, as fused to the leucine zipper domain of c-jun. Figure 14.
MKQEVTQIPAALSVPEGENLVLNCSFTDSAIYNLQWFRQDPGKGLTSLLLIQSSQREQTSGRLNASLDKSSGRSTLYIAASQPGDSATYLCAVTNFNKFYFGSGTKLNVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSPGGRIARLEEKVKTLKAQNSELASTANMLREQVAQLKQKVMNY

SEQ ID NO: 50 - DNA, 864 bp, Artificial Sequence; identical to SEQ ID NO: 46
  Description of Artificial Sequence DNA sequence of soluble HLA-A2/HIV-1 Gag restricted TCRbeta chain from patient 003, as fused to the leucine zipper domain of c-fos. FIGURE 15.

SEQ ID NO: 51 - protein, 287 aa, Artificial Sequence; identical to SEQ ID NO: 47
  Description of Artificial Sequence Amino acid sequence of soluble HLA-A2/HIV-1 Gag restricted TCR beta chain from patient 003, as fused to the leucine zipper domain of c-fos. FIGURE 15.

SEQ ID NO: 52 - DNA, 750 bp, Artificial Sequence
  Description of Artificial Sequence DNA sequence of soluble HTLV1 Tax/HLA-A2 restricted TCR alpha chain clone A6, as fused to the leucine zipper domain of c-jun. FIGURE 16.
atgcagaaggaagtggagcagaactctggacccctcagtgttccagagggagccattgcctctctcaactgcacttacagtgaccgaggttcccagtccttcttctggtacagacaatattctgggaaaagccctgagttgataatgtccatatactccaatggtgacaaagaagatggaaggtttacagcacagctcaataaagccagccagtatgtttctctgctcatcagagactcccagcccagtgattcagccacctacctctgtgccgttacaactgacagctgggggaaattgcagtttggagcagggacccaggttgtggtcaccccagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcccccgggggtagaatcgcccggctggaggaaaaagtgaaaaccttgaaagctcagaactcggagctggcgtccacggccaacatgctcagggaacaggtggcacagcttaaacagaaagtcatgaactactag

SEQ ID NO: 53 - protein, 249 aa, Artificial Sequence
  Description of Artificial Sequence Amino acid sequence of soluble HLA-A2/HTLV-1 Tax restricted TCR alpha chain from clone A6, as fused to the leucine zipper domain of c-jun. FIGURE 16.
MQKEVEQNSGPLSVPEGAIASLNCTYSDRGSQSFFWYRQYSGKSPELIMSIYSNGDKEDGRFTAQLNKASQYVSLLIRDSQPSDSATYLCAVTTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSPGGRIARLEEKVKTLKAQNSELASTANMLREQVAQLKQKVMNY

SEQ ID NO: 54 - DNA, 928 bp, Artificial Sequence
  Description of Artificial Sequence DNA sequence of soluble HLA-A2/HTLV-1 Tax restricted TCR beta chain from clone A6, as fused to the leucine zipper domain of c-fos and a BirA biotinylation tag.
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagaccccgggggtctgactgatacactccaagcggagacagatcaacttgaagacaagaagtctgcgttgcagaccgagattgccaatctactgaaagagaaggaaaaactagagttcatcctggcagcttacggatccggtggtggtctgaacgatatttttgaagctcagaaaatcgaatggcattaagctt

SEQ ID NO: 55 - protein, 307 aa, Artificial Sequence
  Description of Artificial SequenceAmino acid sequence of soluble HLA-A2/HTLV-1 Tax restricted TCR beta chain from clone A6, as fused to the leucine zipper domain of c-fos and a BirA biotinylation tag
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLKNVFPPEVAVFEPSEAEISHTQKATLVCLATGFYPDHVELSWWVNGKEVHSGVSTDPQPLKEQPALNDSRYALSSRLRVSATFWQNPRNHFRCQVQFYGLSENDEWTQDRAKPVTQIVSAEAWGRADPGGLTDTLQAETDQLEDKKSALQTEIANLLKEKEKLEFILAAYGSGGGLNDIFEAQKIEWH

SEQ ID NO: 56 - DNA, 765 bp, Artificial Sequence
  Description of Artificial SequenceDNA sequence of soluble HLA-A2/HTLV-1 Tax restricted TCR alpha chain from clone M10B7/D3 fused to the c-jun leucine zipper domain.
atgcaacagaagaatgatgaccagcaagttaagcaaaattcaccatccctgagcgtccaggaaggaagaatttctattctgaactgtgactatactaacagcatgtttgattatttcctatggtacaaaaaataccctgctgaaggtcctacattcctgatatctataagttccattaaggataaaaatgaagatggaagattcactgtcttcttaaacaaaagtgccaagcacctctctctgcacattgtgccctcccagcctggagactctgcagtgtacttctgtgcagcaatggagggagcccagaagctggtatttggccaaggaaccaggctgactatcaacccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcccccgggggtagaatcgcccggctggaggaaaaagtgaaaaccttgaaagctcagaactcggagctggcgtccacggccaacatgctcagggaacaggtggcacagcttaaacagaaagtcatgaactactag

SEQ ID NO: 57 - protein, 254 aa, Artificial Sequence [sequence omitted]

SEQ ID NO: 58 - DNA, 925 bp, Artificial Sequence
  Description of Artificial SequenceDNA sequence of soluble HLA-A2/HTLV-1 Tax restricted TCR beta chain from clone M10B7/D3 fused to the c-fos leucine zipper domain and a BirA biotinylation tag.
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttaccaggagggggggttttacgagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacccgtcacccagatcgtcagcgccgaggcctggggtagagcagaccccgggggtctgactgatacactccaagcggagacagatcaacttgaagacaagaagtctgcgttgcagaccgagattgccaatctactgaaagagaaggaaaaactagagttcatcctggcagcttacggatccggtggtggtctgaacgatatttttgaagctcagaaaatcgaatggcattaagctt

SEQ ID NO: 59 - protein, 306 aa, Artificial Sequence
  Description of Artificial SequenceAmino acid sequence of soluble HLA-A2/HTLV-1 Tax restricted TCR beta chain from clone M10B7/D3 fused to the c-fos leucine zipper domain and a BirA biotinylation tag.
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYPGGGFYEQYFGPGTRLTVTEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADPGGLTDTLQAETDQLEDKKSALQTEIANLLKEKEKLEFILAAYGSGGGLNDIFEAQKIEWH

SEQ ID NO: 60 - DNA, 928 bp, Artificial Sequence
  Mutated DNA sequence of soluble HLA-A2/HTLV-1 Tax restricted TCR beta chain from clone A6 fused to the c-fos leucine zipper domain and a BirA biotinylation tag.
atgaacgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcaggccgggactagcgggagggcgaccagagcagtacttcgggccgggcaccaggctcacggtcacagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatacgctctgagcagccgcctgagggtctcggccaccttctggcaggacccccgcaaccacttccgctgtcaagtccagttctacgggctctcggagaatgacgagtggacccaggatagggccaaacctgtcacccagatcgtcagcgccgaggcctggggtagagcagaccccgggggtctgactgatacactccaagcggagacagatcaacttgaagacaagaagtctgcgttgcagaccgagattgccaatctactgaaagagaaggaaaaactagagttcatcctggcagcttacggatccggtggtggtctgaacgatatttttgaagctcagaaaatcgaatggcattaagctt

SEQ ID NO: 61 - protein, 307 aa, Artificial Sequence
  Amino acid sequence of mutated soluble HLA-A2/HTLV-1 Tax restricted TCR beta chain from clone A6 fused to the c-fos leucine zipper domain and a BirA biotinylation tag.
MNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASRPGLAGGRPEQYFGPGTRLTVTEDLKNVFPPEVAVFEPSEAEISHTQKATLVCLATGFYPDHVELSWWVNGKEVHSGVSTDPQPLKEQPALNDSRYALSSRLRVSATFWQDPRNHFRCQVQFYGLSENDEWTQDRAKPVTQIVSAEAWGRADPGGLTDTLQAETDQLEDKKSALQTEIANLLKEKEKLEFILAAYGSGGGLNDIFEAQKIEWH

SEQ ID NO: 62 - DNA, 190 bp, Artificial Sequence
  DNA sequence of the c-fos-BirA biotinylation tag fusion partner used for TCR beta chains.
cccgggggtctgactgatacactccaagcggagacagatcaacttgaagacaagaagtctgcgttgcagaccgagattgccaatctactgaaagagaaggaaaaactagagttcatcctggcagcttacggatccggtggtggtctgaacgatatttttgaagctcagaaaatcgaatggcattaagctt

SEQ ID NO: 63 - protein, 61 aa, Artificial Sequence
  Description of Artificial SequenceAmino acid sequence of the c-fos-BirA biotinylation tag fusion partner used for TCR beta chains.
PGGLTDTLQAETDQLEDKKSALQTEIANLLKEKEKLEFILAAYGSGGGLNDIFEAQKIEWH

SEQ ID NO: 64 - DNA, 42 bp, Artificial Sequence
  Reverse primer used for PCR amplification of the v-beta-c-fos leucine zipper fragment of the Influenza Matrix peptide/HLA-A0201 restricted human JM22 TCR fusion gene.
acacacggatccgtaagctgcgacgatgaactcgattttctt

SEQ ID NO: 65 - DNA, 744 bp, Artificial Sequence; identical to SEQ ID NO: 40
  Description of Artificial Sequence Gene coding for human HLA-A2/flu matrix peptide restricted JM22 TCR alpha chain fused to c-jun leucine zipper domain.

SEQ ID NO: 66 - protein, 247 aa, Artificial Sequence; identical to SEQ ID NO: 41
  Description of Artificial Sequence Amino acid sequence of  human HLA-A2/flu matrix peptide restricted JM22 TCR alpha chain fused to c-jun leucine zipper domain.

SEQ ID NO: 67 - DNA, 864 bp, Artificial Sequence; identical to SEQ ID NO: 42
  Description of Artificial Sequence Gene coding for human HLA-A2/flu matrix peptide restricted JM22 TCR beta chain fused to c-fos leucine zipper domain.

SEQ ID NO: 68 - protein, 287 aa, Artificial Sequence; identical to SEQ ID NO: 43
  Description of Artificial Sequence Amino acid sequence of human HLA-A2/flu matrix peptide restricted JM22 TCR beta chain fused to c-fos leucine zipper domain.

SEQ ID NO: 69 - DNA, 918 bp, Artificial Sequence
  Description of Artificial Sequence  Gene coding for human HLA-A2/flu matrix peptide restricted JM22 TCR beta chain fused to c-fos leucine zipper domain and BirA biotinylation tag.
atggtggatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagttcgaggagctcctacgagcagtacttcgggccgggcaccaggctcacggtcacagaggacctgaaaaacgttttcccacccgaggtcgctgtgtttgaaccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagaccccgggggtctgactgatacactccaagcggagacagatcaacttgaagacaagaagtctgcgttgcagaccgagattgccaatctactgaaagagaaggaaaaactagagttcatcctggcagcttacggatccggtggtggtctgaacgatatttttgaagctcagaaaatcgaatggcattaa

SEQ ID NO: 70 - protein, 305 aa, Artificial Sequence
  Description of Artificial Sequence Amino acid sequence of human HLA-A2/flu matrix peptide restricted JM22 TCR beta chain fused to c-fos leucine zipper domain and BirA biotinylation tag.
MVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSSRSSYEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADPGGLTDTLQAETDQLEDKKSALQTEIANLLKEKEKLEFILAAYGSGGGLNDIFEAQKIEWH

SEQ ID NO: 71 - DNA, 750 bp, Artificial Sequence; identical to SEQ ID NO: 52
  Description of Artificial Sequence Gene coding for human HLA-A2/HTLV-1 Tax peptide restricted TCR alpha chain from clone A6 fused to the c-jun leucine zipper domain.

SEQ ID NO: 72 - protein, 249 aa, Artificial Sequence; identical to SEQ ID NO: 53
  Description of Artificial Sequence  Amino acid sequence of human HLA-A2/HTLV-1 Tax restricted TCR alpha chain from clone A6 fused to the c-jun leucine zipper domain.

SEQ ID NO: 73 - DNA, 928 bp, Artificial Sequence; identical to SEQ ID NO: 54
  Description of Artificial Sequence Gene coding for human HLA-A2/HTLV-1 Tax peptide restricted TCR beta chain from clone A6 fused to the c-fos leucine zipper domain and BirA biotinylation tag.

SEQ ID NO: 74 - protein, 307 aa, Artificial Sequence; identical to SEQ ID NO: 55
  Description of Artificial Sequence  Amino acid sequence of human HLA-A2/HTLV-1 Tax restricted TCR beta chain from clone A6 fused to the c-fos leucine zipper domain and BirA biotinylation tag.

SEQ ID NO: 75 - DNA, 765 bp, Artificial Sequence; identical to SEQ ID NO: 56
  Description of Artificial Sequence Gene coding for human HLA-A2/HTLV-1 Tax peptide restricted TCR alpha chain from clone M10B7/D3 fused to the c-jun leucine zipper domain.

SEQ ID NO: 76 - protein, 254 aa, Artificial Sequence [sequence omitted]

SEQ ID NO: 77 - DNA, 925 bp, Artificial Sequence; identical to SEQ ID NO: 58
  Description of Artificial Sequence Gene coding for human HLA-A2/HTLV-1 Tax peptide restricted TCR beta chain from clone M10B7/D3 fused to the c-fos leucine zipper domain and BirA biotinylation tag.

SEQ ID NO: 78 - protein, 306 aa, Influenza virus; identical to SEQ ID NO: 59

SEQ ID NO: 79 - protein, 9 aa, Influenza virus
  Peptide derived from Influenza virus Matrix protein and presented as peptide antigen by HLA-A0201. This HLA/peptide combination restricts the JM22 TCR.
GILGFVFTL

SEQ ID NO: 80 - protein, 9 aa, Human immunodeficiency virus type 1
  Peptide derived fromHIV-1 Gag protein and presented as peptide antigen by HLA-A0201. This HLA/peptide combination restricts the cloned TCR from patient 003.
SLYNTVATL

SEQ ID NO: 81 - protein, 9 aa, Influenza virus
  Peptide derived from Influenza virus nucleoprotein and presented as peptide antigen by the murine H2-Db. This HLA/peptide combination restricts the murine F5 TCR.
ASNENMDAM

SEQ ID NO: 82 - protein, 9 aa, Human T-cell lymphotropic virus type 1
  Peptide derived from HTLV-1 Tax protein and presented as peptide antigen by HLA-A0201. This HLA/peptide combination restricts the A6 and B7 TCRs.
LLFGYPVYV

SEQ ID NO: 83 - protein, 9 aa, Human immunodeficiency virus
  Peptide derived from HIV-1 Pol protein and presented as peptide antigen by HLA-A0201.
ILKEPVHGV

SEQ ID NO: 84 - DNA, 37 bp, Artificial Sequence
  Description of Artificial Sequence Primer for PCR amplification of the human Vbeta17 chain fused to the BirA biotinylation tag.
gctctagacatatgggcccagtggattctggagtcac

SEQ ID NO: 85 - DNA, 90 bp, Artificial Sequence
  Description of Artificial Sequence Primer for PCR amplification of the human Vbeta17 chain fused to the BirA biotinylation tag.
gggggaagcttaatgccattcgattttctgagcttcaaaaatatcgttcagaccaccaccggatccgtaagctgccaggatgaactctag